SEQUENCE LISTING (223 sequences)

SEQ ID NO: 1 - protein, 150 aa, Bos taurus
LPVKPTSSGSSEEKQLNNKYPDAVATWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKK

SEQ ID NO: 2 - protein, 28 aa, Artificial Sequence
  CK2-SS
ELEELNVPGADDSSSSDDDDDDDRINKK

SEQ ID NO: 3 - protein, 28 aa, Artificial Sequence
  CK2-S
ELEELNVPGADDSSSDDDSDDDDRINKK

SEQ ID NO: 4 - protein, 53 aa, Artificial Sequence
  CK2-S-6H
MRELEELNVPGADDSSSDDDSDDDDRINKKIEDPNSSSVDKLAAALEHHHHHH

SEQ ID NO: 5 - protein, 21 aa, Artificial Sequence
  Phosphopeptide 1
EMEAESISSSEEIVPNSVEQK

SEQ ID NO: 6 - protein, 20 aa, Artificial Sequence
  Phosphopeptide 2
ELEELNVPGEIVESLSSSEE

SEQ ID NO: 7 - protein, 20 aa, Artificial Sequence
  Phosphopeptide 3
NTMEHVSSSEESIISQETYK

SEQ ID NO: 8 - protein, 25 aa, Artificial Sequence
  Phosphopeptide 4
NANEEEYSIGSSSEESAEVATEEVK

SEQ ID NO: 9 - protein, 11 aa, Artificial Sequence
  Phosphopeptide 5
EQLSTSEENSK

SEQ ID NO: 10 - protein, 349 aa, Homo sapiens
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCTVFQTQPVTSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 11 - protein, 294 aa, Homo sapiens
ESSSEDVSQEESLFLISGKPEGRRPQGGNQPQRPPPPPGKPQGPPPQGGNQSQGPPPPPGKPEGRPPQGGNQSQGPPPHPGKPERPPPQGGNQSQGPPPHPGKPESRPPQGGHQSQGPPPTPGKPEGPPPQGGNQSQGTPPPPGKPEGRPPQGGNQSQGPPPHPGKPERPPPQGGNQSHRPPPPPGKPERPPPQGGNQSQGPPPHPGKPEGPPPQEGNKSRSARSPPGKPQGPPQQEGNKPQGPPPPGKPQGPPPPGGNPQQPQAPPAGKPQGPPPPPQGGRPPRPAQGQQPPQ

SEQ ID NO: 12 - protein, 84 aa, Homo sapiens
YESHESMESYELNPFINRRNANTFISPQQRWRAKVQERIRERSKPVHELNREACDDYRLCERYAMVYGYNAAYNRYFRKRRGTK

SEQ ID NO: 13 - protein, 211 aa, Homo sapiens
MISRMEKMTMMMKILIMFALGMNYWSCSGFPVYDYDPSSLRDALSASVVKVNSQSLSPYLFRAFRSSLKRVEVLDENNLVMNLEFSIRETTCRKDSGEDPATCAFQRDYYVSTAVCRSTVKVSAQQVQGVHARCSWSSSTSESYSSEEMIFGDMLGSHKWRNNYLFGLISDESISEQFYDRSLGIMRRVLPPGNRRYPNHRHRARINTDFE

SEQ ID NO: 14 - protein, 222 aa, Gallus gallus
CQQYGCLEGDTHKANPSPEPNMHECTLYSESSCCYANFTEQLAHSPIIKVSNSYWNRCGQLSKSCEDFTKKIECFYRCSPHAARWIDPRYTAAIQSVPLCQSFCDDWYEACKDDSICAHNWLTDWERDESGENHCKSKCVPYSEMYANGTDMCQSMWGESFKVSESSCLCLQMNKKDMVAIKHLLSESSEESSSMSSSEEHACQKKLLKFEALQQEEGEERR

SEQ ID NO: 15 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 16 - protein, 317 aa, Homo sapiens
MKTALILLSILGMACAFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGVDNGEEGEEESVTGANAEGTTETGGQGKGTSKTTTSPNGGFEPTTPPQVYRTTSPPFGKTTTVEYEGEYEYTGANDYDNGYEIYESENGEPRGDNYRAYEDEYSYFKGQGYDGYDGQNYYHHQ

SEQ ID NO: 17 - protein, 525 aa, Homo sapiens
MRVFCVGLLLFSVTWAAPTFQPQTEKTKQSCVEEQRQEEKNKDNIGFHHLGKRINQELSSKENIVQERKKDLSLSEASENKGSSKSQNYFTNRQRLNKEYSISNKENTHNGLRMSIYPKSTGNKGFEDGDDAISKLHDQEEYGAALIRNNMQHIMGPVTAIKLLGEENKENTPRNVLNIIPASMNYAKAHSKDKKKPQRDSQAQKSPVKSKSTHRIQHNIDYLKHLSKVKKIPSDFEGSGYTDLQERGDNDISPFSGDGQPFKDIPGKGEATGPDLEGKDIQTGFAGPSEAESTHLDTKKPGYNEIPEREENGGNTIGTRDETAKEADAVDVSLVEGSNDIMGSTNFKELPGREGNRVDAGSQNAHQGKVEFHYPPAPSKEKRKEGSSDAAESTNYNEIPKNGKGSTRKGVDHSNRNQATLNEKQRFPSKGKSQGLPIPSRGLDNEIKNEMDSFNGPSHENIITHGRKYHYVPHRQNNSTRNKGMPQGKGSWGRQPHSNRRFSSRRRDDSSESSDSGSSSESDGD

SEQ ID NO: 18 - protein, 513 aa, Homo sapiens
MKISILLMFLWGLSCALPVTRYQNNESEDSEEWKGHLAQAPTPPLESSESSEGSKVSSEEQANEDPSDSTQSEEGLGSDDHQYIYRLAGGFSRSTGKGGDDKDDDEDDSGDDTFGDDDSGPGPKDRQEGGNSRLGSDEDSDDTIQASEESAPQGQDSAQDTTSESRELDNEDRVDSKPEGGDSTQESESEEHWVGGGSDGESSHGDGSELDDEGMQSDDPESIRSERGNSRMNSAGMKSKESGENSEQANTQDSGGSQLLEHPSRKIFRKSRISEEDDRSELDDNNTMEEVKSDSTENSNSRDTGLSQPRRDSKGDSQEDSKENLSQEESQNVDGPSSESSQEANLSSQENSSESQEEVVSESRGDNPDPTTSYVEDQEDSDSSEEDSSHTLSHSKSESREEQADSESSESLNFSEESPESPEDENSSSQEGLQSHSSSAESQSEESHSEEDDSDSQDSSRSKEDSNSTESKSSSEEDGQLKNIEIESRKLTVDAYHNKPIGDQDDNDCQDGY

SEQ ID NO: 19 - protein, 27 aa, Artificial Sequence
  modified bovine beta-casein C-terminal sequence extended
PIIVPRDPNSSSVDKLAAALEHHHHHH

SEQ ID NO: 20 - protein, 12 aa, Artificial Sequence
  phosphate centre sequence of CK2-S-6H peptide fragment with 2, 3 or 4 phosphorylated residues
DDSSSDDDSDDD

SEQ ID NO: 21 - protein, 12 aa, Artificial Sequence
  CK2-S-6H unphosphorylated peptide fragment
KIEDPNSSSVDK

SEQ ID NO: 22 - protein, 9 aa, Artificial Sequence
  CK2-S-6H unphosphorylated peptide fragment
IEDPNSSDK

SEQ ID NO: 23 - protein, 18 aa, Bos taurus
LPVKPTSSGSSEEKQLNN

SEQ ID NO: 24 - protein, 11 aa, Bos taurus
QNSVSSEETDD

SEQ ID NO: 25 - protein, 17 aa, Bos taurus
PDHSDESHHSDESDEVD

SEQ ID NO: 26 - protein, 18 aa, Mus musculus
ARYHNTESESSEERTDDL

SEQ ID NO: 27 - protein, 12 aa, Mus musculus
PTNSESSEESQA

SEQ ID NO: 28 - protein, 17 aa, Mus musculus
GQANSDHTDSSESGEEL

SEQ ID NO: 29 - protein, 19 aa, Mus musculus
SDEDSADTTQSSEDSTSQE

SEQ ID NO: 30 - protein, 14 aa, Mus musculus
DTPSDSKDQDSEDE

SEQ ID NO: 31 - protein, 10 aa, Mus musculus
DSAQDSESEE

SEQ ID NO: 32 - protein, 17 aa, Cavia porcellus
RGGSSSSSSSEERLKEE

SEQ ID NO: 33 - protein, 11 aa, Cavia porcellus
IISESTEEREA

SEQ ID NO: 34 - protein, 17 aa, Cavia porcellus
QREASSISSSEEVVPKN

SEQ ID NO: 35 - protein, 17 aa, Sus scrofa
KHEMEHVSSSEESINIS

SEQ ID NO: 36 - protein, 11 aa, Sus scrofa
ASSSSSEESVD

SEQ ID NO: 37 - protein, 17 aa, Sus scrofa
IQSGEELSTSEEPVSSS

SEQ ID NO: 38 - protein, 17 aa, Homo sapiens
RETIESLSSSEESITEY

SEQ ID NO: 39 - protein, 16 aa, Homo sapiens
SSEENGDDSSEEEEEE

SEQ ID NO: 40 - protein, 16 aa, Homo sapiens
SSESSDSGSSSESDGD

SEQ ID NO: 41 - protein, 15 aa, Homo sapiens
GVVSLGSPSGEVSHP

SEQ ID NO: 42 - protein, 16 aa, Homo sapiens
SSSTSESYSSEEMIFG

SEQ ID NO: 43 - protein, 11 aa, Homo sapiens
YESHESMESYE

SEQ ID NO: 44 - protein, 12 aa, Homo sapiens
ESSSEDVSQEES

SEQ ID NO: 45 - protein, 19 aa, Gallus gallus
SESSEESSSMSSSEEHACQ

SEQ ID NO: 46 - protein, 19 aa, Artificial Sequence
  CK2-SS
ELNVPGADDSSSSDDDDDD

SEQ ID NO: 47 - protein, 19 aa, Artificial Sequence
  CK2-S
ELNVPGADDSSSDDDSDDD

SEQ ID NO: 48 - protein, 28 aa, Bos taurus
RELEELNVPGEIVESLSSSEESITRINK

SEQ ID NO: 49 - protein, 28 aa, Bubalus bubalis
RELEELNVPGEIVESLSSSEESITHINK

SEQ ID NO: 50 - protein, 28 aa, Camelus dromedarius
REKEEFKTAGEALESISSSEESITHINK

SEQ ID NO: 51 - protein, 28 aa, Ovis aries
REQEELNVVGETVESLSSSEESITHINK

SEQ ID NO: 52 - protein, 28 aa, Capra hircus
REQEELNVVGETVESLSSSEESITHINK

SEQ ID NO: 53 - protein, 34 aa, Equus caballus
REKEELNVSSETVESLSSNEPDSSSEESITHINK

SEQ ID NO: 54 - protein, 28 aa, Canis familiaris
REKEELTLSNETVESLSSSEESITHINK

SEQ ID NO: 55 - protein, 27 aa, Oryctolagus cuniculus
REKEQLSVPTEAVGSVSSSEEITHINK

SEQ ID NO: 56 - protein, 28 aa, Sus scrofa
RAKEELNASGETVESLSSSEESITHISK

SEQ ID NO: 57 - protein, 25 aa, Mus musculus
RETTFTVSSETDSISSEESVEHINE

SEQ ID NO: 58 - protein, 26 aa, Rattus norvegicus
REKDAFTVSSETGSISSEESVEHINE

SEQ ID NO: 59 - protein, 19 aa, Homo sapiens
RETIESLSSSEESITEYKQ

SEQ ID NO: 60 - protein, 19 aa, Macropus eugenii
RPMVEKISESEEYVNEVPE

SEQ ID NO: 61 - protein, 19 aa, Trichosurus vulpecula
RPMVEKISESEEHVTDVPE

SEQ ID NO: 62 - protein, 24 aa, Bos taurus
KHPIKHQGLPQEVLNENLLRFFVA

SEQ ID NO: 63 - protein, 24 aa, Bubalus bubalis
KQPIKHQGLPQGVLNENLLRFFVA

SEQ ID NO: 64 - protein, 24 aa, Ovis aries
KHPIKHQGLSPEVLNENLLRFVVA

SEQ ID NO: 65 - protein, 24 aa, Capra hircus
KHPINHQGLSPEVLNENLLRFVVA

SEQ ID NO: 66 - protein, 33 aa, Camelus dromedarius
KYPLRYPEVFQNEPDSIEEVLNKRKILELAVVS

SEQ ID NO: 67 - protein, 32 aa, Sus scrofa
KPPLRHQEHLQNEPDSREELFKERKFLRFPEV

SEQ ID NO: 68 - protein, 32 aa, Oryctolagus cuniculus
KFHLGHLKLTQEQPESSEQEILKERKLLRFVQ

SEQ ID NO: 69 - protein, 37 aa, Cavia porcellus
KFPFRHTELFQTQRGGSSSSSSSEERLKEENIFKFDQ

SEQ ID NO: 70 - protein, 35 aa, Rattus norvegicus
RAHRRNAVSSQTQQENSSSEEQEIVKQPKYLSLNE

SEQ ID NO: 71 - protein, 33 aa, Mus musculus
RLHSRNAVSSQTQQQHSSSEEIFKQPKYLNLNQ

SEQ ID NO: 72 - protein, 18 aa, Homo sapiens
KLPLRYPERLQNPSESSE

SEQ ID NO: 73 - protein, 35 aa, Macropus eugenii
RELENRLNEDPIPVSEASSSEESVHQLNRDRRPLE

SEQ ID NO: 74 - protein, 24 aa, Bos taurus
EKVNELSKDIGSESTEDQAMEDIK

SEQ ID NO: 75 - protein, 24 aa, Bubalus bubalis
EKVNELSTDIGSESTEDQAMEDIK

SEQ ID NO: 76 - protein, 24 aa, Ovis aries
ENINELSKDIGSESIEDQAMEDAK

SEQ ID NO: 77 - protein, 24 aa, Capra hircus
ENINELSKDIGSESTEDQAMEDAK

SEQ ID NO: 78 - protein, 23 aa, Camelus dromedarius
ENIDELKDTRNEPTEDHIMEDTE

SEQ ID NO: 79 - protein, 8 aa, Sus scrofa
EIINELNR

SEQ ID NO: 80 - protein, 24 aa, Oryctolagus cuniculus
EKENEEIKGTRNEVTEEHVLADRE

SEQ ID NO: 81 - protein, 16 aa, Cavia porcellus
KQSEKIKEIISESTEE

SEQ ID NO: 82 - protein, 23 aa, Rattus norvegicus
EQDNEIKITMDSSAEEQATASAQ

SEQ ID NO: 83 - protein, 23 aa, Mus musculus
EQNDEIKVTMDAASEEQAMASAQ

SEQ ID NO: 84 - protein, 24 aa, Homo sapiens
EKQTDEIKDTRNESTQNCVVAEPE

SEQ ID NO: 85 - protein, 24 aa, Macropus eugenii
YELDKYREDLKTSSSEEFVTPSTN

SEQ ID NO: 86 - protein, 19 aa, Bos taurus
QMEAESISSSEEIVPNSVE

SEQ ID NO: 87 - protein, 19 aa, Bubalus bubalis
QMEAESISSSEEIVPISVE

SEQ ID NO: 88 - protein, 19 aa, Ovis aries
QMKAGSSSSSEEIVPNSAE

SEQ ID NO: 89 - protein, 19 aa, Capra hircus
QMKAGSSSSSEEIVPNSAE

SEQ ID NO: 90 - protein, 18 aa, Camelus dromedarius
RKESGSSSSEEVVSSTTE

SEQ ID NO: 91 - protein, 18 aa, Sus scrofa
QRGSSSSSSEEVVGNSAE

SEQ ID NO: 92 - protein, 18 aa, Oryctolagus cuniculus
TEASISSSSEEIVPSSTK

SEQ ID NO: 93 - protein, 19 aa, Cavia porcellus
QREASSISSSEEVVPKNTE

SEQ ID NO: 94 - protein, 22 aa, Rattus norvegicus
EDSSSSSSSSEESKDAIPSATE

SEQ ID NO: 95 - protein, 21 aa, Mus musculus
EDSSISSSSEESEEAIPNITE

SEQ ID NO: 96 - protein, 19 aa, Homo sapiens
KMESSISSSSEEMSLSKCA

SEQ ID NO: 97 - protein, 20 aa, Macropus eugenii
NFNEEDSSASRERKIEDFSE

SEQ ID NO: 98 - protein, 18 aa, Bos taurus
KNTMEHVSSSEESIISQE

SEQ ID NO: 99 - protein, 19 aa, Ovis aries
KHKMEHVSSSEEPINISQE

SEQ ID NO: 100 - protein, 19 aa, Capra hircus
KHKMEHVSSSEEPINIFQE

SEQ ID NO: 101 - protein, 19 aa, Camelus dromedarius
KHEMDQGSSSEESINVSQQ

SEQ ID NO: 102 - protein, 19 aa, Sus scrofa
KHEMEHVSSSEESINISQE

SEQ ID NO: 103 - protein, 18 aa, Oryctolagus cuniculus
KPKIEQSSSEETIAVSQE

SEQ ID NO: 104 - protein, 19 aa, Oryctolagus cuniculus
KNGIEQRSASEEIVSFYQE

SEQ ID NO: 105 - protein, 19 aa, Mus musculus
KHEIKDKSSSEESSASIYP

SEQ ID NO: 106 - protein, 19 aa, Mus musculus
KQRMEQYISSEESMDNSQE

SEQ ID NO: 107 - protein, 19 aa, Cavia porcellus
KHKSEQQSSSEESVSISQE

SEQ ID NO: 108 - protein, 19 aa, Rattus norvegicus
KHAVKDKPSSEESASVYLG

SEQ ID NO: 109 - protein, 26 aa, Bos taurus
EVVRNANEEEYSIGSSSEESAEVATE

SEQ ID NO: 110 - protein, 26 aa, Ovis aries
EVVRNADEEEYSIRSSSEESAEVAPE

SEQ ID NO: 111 - protein, 26 aa, Capra hircus
EVVRNANEEEYSIRSSSEESAEVAPE

SEQ ID NO: 112 - protein, 17 aa, Camelus dromedarius
EAVRNIKEVESAEVPTE

SEQ ID NO: 113 - protein, 26 aa, Sus scrofa
EAVRNIKEVGYASSSSSEESVDIPAE

SEQ ID NO: 114 - protein, 17 aa, Oryctolagus cuniculus
EPIKNINEVEYVEVPTE

SEQ ID NO: 115 - protein, 18 aa, Oryctolagus cuniculus
QETPSVSSSEESVEVQTE

SEQ ID NO: 116 - protein, 17 aa, Mus musculus
DSASSSSSEESSEEVSE

SEQ ID NO: 117 - protein, 9 aa, Mus musculus
ETVENIYIP

SEQ ID NO: 118 - protein, 26 aa, Cavia porcellus
EATKNTPKMAFFSRSSSEEFADIHRE

SEQ ID NO: 119 - protein, 17 aa, Rattus norvegicus
DSASSSSSEESSEEISE

SEQ ID NO: 120 - protein, 18 aa, Bos taurus
NREQLSTSEENSKKTVDM

SEQ ID NO: 121 - protein, 18 aa, Ovis aries
NREQLSTSEENSKKTIDM

SEQ ID NO: 122 - protein, 18 aa, Capra hircus
NREQLSTSEENSKKTIDM

SEQ ID NO: 123 - protein, 17 aa, Camelus dromedarius
NTEQLSISEESTEVPTE

SEQ ID NO: 124 - protein, 25 aa, Sus scrofa
SGEELSTSEEPVSSSQEENTKTVDM

SEQ ID NO: 125 - protein, 17 aa, Mus musculus
ESISTSVEEILKKIIDM

SEQ ID NO: 126 - protein, 17 aa, Cavia porcellus
ESSSSSSTEKSTDVFIK

SEQ ID NO: 127 - protein, 17 aa, Rattus norvegicus
ESTSTSVEEILKKIIDI

SEQ ID NO: 128 - protein, 24 aa, Homo sapiens
GSSDSSEENGDDSSEEEEEEEETS

SEQ ID NO: 129 - protein, 25 aa, Rattus norvegicus
GGSDSSEENGDGDSSEEEGEEEETS

SEQ ID NO: 130 - protein, 25 aa, Mus musculus
GGSDSSEENGDGDSSEEEGEEEETS

SEQ ID NO: 131 - protein, 25 aa, Bos taurus
SSSDSSEENGNGDSSEEEEEEEETS

SEQ ID NO: 132 - protein, 25 aa, Sus scrofa
SSSDSSEENGNGDSSEEEEEEEENS

SEQ ID NO: 133 - protein, 22 aa, Gallus gallus
GSDSSEEEGDGSEEEEEGGAPS

SEQ ID NO: 134 - protein, 13 aa, Bos taurus
VKPTSSGSSEEKQ

SEQ ID NO: 135 - protein, 14 aa, Bos taurus
NSVSSEETDDNKQN

SEQ ID NO: 136 - protein, 24 aa, Bos taurus
SDDAETTDDPDHSDESHHSDESDE

SEQ ID NO: 137 - protein, 13 aa, Ovis aries
VKPTSSGSSEEKQ

SEQ ID NO: 138 - protein, 14 aa, Ovis aries
NSVSSEETDDNKQN

SEQ ID NO: 139 - protein, 24 aa, Ovis aries
SDDAETPDDSDHSNESHHSDESDE

SEQ ID NO: 140 - protein, 13 aa, Sus scrofa
VKQTNSGSSEEKL

SEQ ID NO: 141 - protein, 14 aa, Sus scrofa
NTISSEETDDFKQE

SEQ ID NO: 142 - protein, 24 aa, Sus scrofa
SEEADHADDADRSDESHHSDESDE

SEQ ID NO: 143 - protein, 13 aa, Oryctolagus cuniculus
VKHADSGSSEEKQ

SEQ ID NO: 144 - protein, 14 aa, Oryctolagus cuniculus
QNMSSEEKDDLKQE

SEQ ID NO: 145 - protein, 24 aa, Oryctolagus cuniculus
SDDADHPDDSHHSDESHQSDESDE

SEQ ID NO: 146 - protein, 12 aa, Rattus norvegicus
VKVAEFGSSEEK

SEQ ID NO: 147 - protein, 14 aa, Rattus norvegicus
NSVSSEETDDFKQE

SEQ ID NO: 148 - protein, 15 aa, Rattus norvegicus
SDESDESHHSDESDE

SEQ ID NO: 149 - protein, 12 aa, Mus musculus
VKVTDSGSSEEK

SEQ ID NO: 150 - protein, 14 aa, Mus musculus
NAVSSEEKDDFKQE

SEQ ID NO: 151 - protein, 15 aa, Mus musculus
SDESDESHHSDESDE

SEQ ID NO: 152 - protein, 13 aa, Homo sapiens
VKQADSGSSEEKQ

SEQ ID NO: 153 - protein, 14 aa, Homo sapiens
NAVSSEETNDFKQE

SEQ ID NO: 154 - protein, 24 aa, Homo sapiens
SNDSDDVDDTDDSHQSDESHHSDE

SEQ ID NO: 155 - protein, 17 aa, Gallus gallus
VSKSRQHAISASSEEKY

SEQ ID NO: 156 - protein, 15 aa, Gallus gallus
THYSSEENADVPEQP

SEQ ID NO: 157 - protein, 11 aa, Gallus gallus
NDSNDTDESDE

SEQ ID NO: 158 - protein, 56 aa, Bos taurus
VARYQNTESKSSEEWKGHLAQTPTPPLESSESSEESKLSSEEQANEDPSDSTESEE

SEQ ID NO: 159 - protein, 56 aa, Homo sapiens
VTRYQNNESEDSEEWKGHLAQAPTPPLESSESSEGSKVSSEEQANEDPSDSTQSEE

SEQ ID NO: 160 - protein, 56 aa, Mus musculus
VARYHNTESESSEERTDDLAGSPPPPTNSESSEESQASPEGQANSDHTDSSESGEE

SEQ ID NO: 161 - protein, 41 aa, Rattus norvegicus
VARYQNTESESSEERTGNLAQSPPPPMANSDHTDSSESGEE

SEQ ID NO: 162 - protein, 62 aa, Bos taurus
GDSRLGSDEDSADTTRSREDSTPQGDEGARDTTSESRDLDREDEGNSRPEGGDSTPDSDSEE

SEQ ID NO: 163 - protein, 62 aa, Homo sapiens
GNSRLGSDEDSDDTIQASEESAPQGQDSAQDTTSESRELDNEDRVDSKPEGGDSTQESESEE

SEQ ID NO: 164 - protein, 61 aa, Mus musculus
GPSKLDSDEDSADTTQSSEDSTSQENSAQDTPSDSKDQDSEDEADSRPEAGDSAQDSESEE

SEQ ID NO: 165 - protein, 60 aa, Rattus norvegicus
GPSRLDSDEDSADTTQSSEDSTSQENSAQDTPSDSKDHHSDEADSRPEAGDSTQDSESEE

SEQ ID NO: 166 - protein, 20 aa, Macaca fascicularis
RREDSSESSDSGSSSESDGD

SEQ ID NO: 167 - protein, 20 aa, Homo sapiens
RRDDSSESSDSGSSSESDGD

SEQ ID NO: 168 - protein, 19 aa, Mus musculus
QRDSSESSSSGSSSESHGD

SEQ ID NO: 169 - protein, 19 aa, Rattus norvegicus
QRDSSESSSSGSSSESSGD

SEQ ID NO: 170 - protein, 13 aa, Bos taurus
FSGVASVESSSGE

SEQ ID NO: 171 - protein, 13 aa, Ovis aries
FSGVASVESASGE

SEQ ID NO: 172 - protein, 13 aa, Sus scrofa
FSGVASVESASGE

SEQ ID NO: 173 - protein, 13 aa, Homo sapiens
FMGVVSLGSPSGE

SEQ ID NO: 174 - protein, 13 aa, Mus musculus
FSPVASVESASGE

SEQ ID NO: 175 - protein, 13 aa, Rattus norvegicus
FSPVASVESASGE

SEQ ID NO: 176 - protein, 13 aa, Pan troglodytes
FMGVVSLGSPSGE

SEQ ID NO: 177 - protein, 10 aa, Cavia porcellus
FPDVDSASGE

SEQ ID NO: 178 - protein, 13 aa, Oryctolagus cuniculus
FAGVPSMESGSGE

SEQ ID NO: 179 - protein, 13 aa, Meriones unguiculatus
FSPVASVESASGE

SEQ ID NO: 180 - protein, 15 aa, Bos taurus
CHWSSSSGSSSSEEM

SEQ ID NO: 181 - protein, 15 aa, Ovis aries
CHWSSSSGSSSSEEM

SEQ ID NO: 182 - protein, 15 aa, Sus scrofa
CRWSSSSESNSSEEM

SEQ ID NO: 183 - protein, 16 aa, Homo sapiens
CSWSSSTSESYSSEEM

SEQ ID NO: 184 - protein, 15 aa, Mus musculus
CRWASSSESNSSEEM

SEQ ID NO: 185 - protein, 15 aa, Rattus norvegicus
CRWASTSESNSSEEM

SEQ ID NO: 186 - protein, 16 aa, Gallus gallus
CHQSTFSSESMSSEEM

SEQ ID NO: 187 - protein, 15 aa, Oncorhynchus mykiss
CGQDSSSSESSSEEN

SEQ ID NO: 188 - protein, 15 aa, Salmo salar
CGQDSSSSESSSEEN

SEQ ID NO: 189 - protein, 11 aa, Bos taurus
YESHESLESYE

SEQ ID NO: 190 - protein, 11 aa, Sus scrofa
YESHESLESYE

SEQ ID NO: 191 - protein, 11 aa, Homo sapiens
YESHESMESYE

SEQ ID NO: 192 - protein, 11 aa, Mus musculus
YESHESMESYE

SEQ ID NO: 193 - protein, 11 aa, Rattus norvegicus
YESHESMESYE

SEQ ID NO: 194 - protein, 11 aa, Oryctolagus cuniculus
YESHESMESYE

SEQ ID NO: 195 - protein, 11 aa, Gallus gallus
YESHESMESHE

SEQ ID NO: 196 - protein, 11 aa, Pongo abelii
YESHESMESYE

SEQ ID NO: 197 - protein, 11 aa, Chinchilla laniger
YESHESMESYE

SEQ ID NO: 198 - protein, 11 aa, Argyrosomus regius
YESHESSESAE

SEQ ID NO: 199 - protein, 10 aa, Galeorhinus galeus
DSSESNEIED

SEQ ID NO: 200 - protein, 19 aa, Homo sapiens
ESSSEDVSQEESLFLISGK

SEQ ID NO: 201 - protein, 19 aa, Gallus gallus
SESSEESSSMSSSEEHACQ

SEQ ID NO: 202 - protein, 55 aa, Bos taurus
RELEELNVPGEIVESLSSSEESITRINKKIEKFQSEEQQQTEDELQDKIHPFAQT

SEQ ID NO: 203 - protein, 56 aa, Artificial Sequence
  CK2-smart beta-casein Site-directed mutagenesis of Serines and neighbouring residues within the phosphate centre of beta-casein results in CK2 smart- or CK2 smarter- sequences
MRELEELNVPGADDSSSDDDSDDDDRINKKIEKFQSEEQQQTEDELQDKIHPFAQT

SEQ ID NO: 204 - protein, 56 aa, Artificial Sequence
  CK2-smarter beta-casein Site-directed mutagenesis of Serines and neighbouring residues within the phosphate centre of beta-casein results in CK2 smart- or CK2 smarter- sequences
MRELEELNVPGADDSSSSDDDDDDDRINKKIEKFQSEEQQQTEDELQDKIHPFAQT

SEQ ID NO: 205 - protein, 53 aa, Artificial Sequence; identical to SEQ ID NO: 4
  CK2-smart beta-casein with 6-HIS-tag

SEQ ID NO: 206 - protein, 138 aa, Artificial Sequence
  CK2-smart repeat multiple repeats of the CK2-S sequence separated by tryptic cleavage sites
MKETAAAKFERQHMDSPDLGTLVPRGSMGYLWIRPGRELEELNVPGADDSSSDDDSDDDDRINKKIEGGGRELEELNVPGADDSSSDDDSDDDDRINKKIEGGGRELEELNVPGADDSSSDDDSDDDDRINKKIEGGG

SEQ ID NO: 207 - protein, 365 aa, Artificial Sequence
  GSTetc+CK2-S chimera sequence CK2-smart phosphate centre cloned into a GST chimera
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGIEGRGSMGYRGSEFCTGLGAPAARGIRPRHMRELEELNVPGADDSSSDDDSDDDDRINKKIEDPPGLERPPPRWSSVDKLAAALEHHHHHHHH

SEQ ID NO: 208 - protein, 321 aa, Artificial Sequence
  hopN A cloned coding sequence for human osteopontin A (hOPN-A) with 6-HIS-tag
MVPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNRDPNSSSVDKLAAALGHHHHHH

SEQ ID NO: 209 - protein, 5 aa, Bos taurus
RELEE

SEQ ID NO: 210 - protein, 4 aa, Artificial Sequence
  sequence having two primary G-CK recognition sites
XXXX

SEQ ID NO: 211 - protein, 4 aa, Artificial Sequence
  primary recognition sequence of CK2
XXXX

SEQ ID NO: 212 - protein, 6 aa, Artificial Sequence
  triplet of vicinal phosphorylated residues
XXXXXX

SEQ ID NO: 213 - protein, 6 aa, Artificial Sequence
  canonical phosphate centre amino acid motif in eutherian beta-caseins
SXSSEE

SEQ ID NO: 214 - protein, 5 aa, Artificial Sequence
  phosphate centre motif in eutherian beta-caseins
SXXEE

SEQ ID NO: 215 - protein, 5 aa, Artificial Sequence
  PC-3 of the eutherian alphaS1-caseins
SXXEE

SEQ ID NO: 216 - protein, 4 aa, Artificial Sequence
  PC-2 of alphaS2-caseins
XEEX

SEQ ID NO: 217 - protein, 6 aa, Artificial Sequence
  PC-1 of OPN
XXSSEE

SEQ ID NO: 218 - protein, 7 aa, Artificial Sequence
  OPN PC-2
SSEETDD

SEQ ID NO: 219 - protein, 13 aa, Artificial Sequence
  MEPE phosphate centre
SSESSXSGSSSEX

SEQ ID NO: 220 - protein, 4 aa, Artificial Sequence
  SPP-24 phosphate centre minimum motif
SSEE

SEQ ID NO: 221 - protein, 5 aa, Artificial Sequence
  PRB4 motif
SSSED

SEQ ID NO: 222 - protein, 9 aa, Artificial Sequence
  Osteopontin phosphate centre 3 (OPN PC-3), Fetuin A and matrix Gla Protein A consensus motif for this type of phosphate centre is (pSXE)3
SXESXESXE

SEQ ID NO: 223 - protein, 7 aa, Artificial
  competent phosphate centre
SXSSSEE

(19) United States
(12) Patent Application Publication
(10) Pub. No.: US 2011/0117206 A1
(43) Pub. Date: May 19, 2011

(54) PHOSPHOPEPTIDES AND USE OF THE SAME

(75) Inventors: Carl Holt, Troon Ayrshire (GB); Roger Clegg, Ayr Ayrshire (GB)

(73) Assignee: PLANT BIOSCIENCE LIMITED, Norwich (GB)

(21) Appl. No.: 13/054,027

(22) PCT Filed: Jul. 16, 2009
(86) PCT No.: PCT/GB2009/050876

§ 371 Date: Jan. 13, 2011

Related U.S. Application Data
(60) Provisional application No. 61/194,311, filed on Sep. 26, 2008.

(30) Foreign Application Priority Data
Jul. 16, 2008 (GB) 0813015.5

(51) Int. Cl. A61K 9/14; A61K 33/42; C07K 2/00; C07K 14/00; A61P 43/00

(52) U.S. Cl. 424/499; 424/602; 530/352; 530/300; 530/324

(57) ABSTRACT

The present application relates to methods of providing thermodynamically stable calcium phosphate nanoclusters and uses thereof.

### FIELD OF THE INVENTION

The present invention relates to methods of providing thermodynamically stable calcium phosphate nanoclusters, in particular to thermodynamically stable nanoclusters with enhanced calcium phosphate sequestering power, phosphopeptides for use in the same and the use of said nanoclusters.

### BACKGROUND OF THE INVENTION

One of the basic physico-chemical problems is how to maintain a state of supersaturation around a mineralised tissue, while inhibiting or preventing mineralization in soft tissues and circulating fluids. Caseins are understood to have the function in milk of sequestering calcium phosphate through clusters of phosphorylated residues in the sequences of α_{S1}-, α_{S2}- and β-caseins. According to the current model of casein micelle structure, the caseins occur as a distribution of roughly spherical particles with a modal radius of about 100 nm. Embedded in the protein matrix of the micelle are more electron dense particles of calcium phosphate, a few nm in radius and linked to the protein matrix through phosphorylated casein residues. Certain casein phosphopeptides have been shown to sequester a precipitate of amorphous calcium phosphate to form thermodynamically stable nanoparticles known as calcium phosphate nanoclusters (CPNs).

In particular, a 25 residue N-terminal β-casein tryptic phosphopeptide (β-casein 1-25) has been determined to form a calcium phosphate nanocluster which comprises a core of amorphous, acidic and hydrated calcium phosphate with a radius of 2.4 nm surrounded by a shell of 50 phosphopeptides with a thickness of 1.6 nm. Although much work has been done on the effects of other secreted phosphoproteins and on calcium phosphate chemistry, the inventors are not aware of any other reports which have been published of equilibrium nanoclusters formed by non-casein secreted phosphoproteins. Whilst a mineral complex with the protein DMP1 (dentin matrix phosphoprotein 1) has been confusingly named a nanocluster by He et al. these are non-equilibrium particles (He, G., Gajjeraman, S., Schultz, D., Cookson, D., Qin, C. L., Butler, W. T., Hao, J. J. & George, A. (2005). Spatially and temporally controlled biomineralization is facilitated by interaction between self-assembled dentin matrix protein 1 and calcium phosphate nuclei in solution. Biochemistry 44, 16140-16148). U.S. Pat. No. 5,227,154 indicates the use of casein phosphopeptides for controlling dental calculus, wherein the specific casein phosphopeptides contain 5 to 40 amino acids, and can be extracted from a casein digest. U.S. Pat. No. 5,015,628 indicates the use of phosphopeptides in relation to caries and gingivitis wherein the phosphopeptides have from 5 to 30 amino acids including the sequence A-B-C-D-E where A, B, C, D and E are independently phosphoserine, phosphothreonine, phosphotyrosine, phosphohistidine, glutamate and aspartate and indicates that in view of cost considerations, it is more economic to extract the phosphopeptide from casein.

### SUMMARY OF THE INVENTION

The present inventors have now determined features of phosphoproteins or phosphopeptides which can form nanoclusters which allow the design of nanoclusters with modified core-shell structures to those nanoclusters previously known. In particular, the present inventors have determined that the core of a calcium phosphate nanocluster can be varied in size depending on the nature of the sequestering phosphopeptide. Advantageously, this can allow for the production of a nanocluster with an increased core size which can sequester a greater amount of amorphous calcium phosphate.

According to a first aspect of the present invention there is provided a method of providing a thermodynamically stable calcium phosphate nanocluster comprising the step of:

preparing a nanocluster forming solution, wherein the nanocluster forming solution is prepared by mixing of calcium ions, phosphate ions and phosphopeptide or phosphoprotein,
wherein the phosphoprotein or phosphopeptide comprises at least one of:
a) a recombinantly expressed phosphopeptide wherein said recombinantly expressed phosphopeptide
   i) includes a phosphate centre modified such that at least one of a phosphorylated residue and an acidic residue or combinations of these residues is increased within the phosphate centre such that the phosphate centre has increased calcium phosphate sequestering power, or
   ii) is modified such that the modified recombinantly expressed phosphopeptide includes an increased number of discrete phosphate centres in comparison to a non-modified version of the recombinantly expressed phosphopeptide, or
   iii) is modified such that the modified recombinant phosphopeptide has increased calcium phosphate sequestering power over a non-modified version of the recombinantly expressed phosphopeptide, or
b) a calcium binding phosphoprotein/phosphopeptide, or a variant or a fragment thereof wherein the phosphopeptide or phosphoprotein does not include whole individual casein or a mixture of caseins or enzymatic digests of an individual casein or mixture of caseins, or
c) a combination of a) and b).

In embodiments, the sequestering power of the peptide may be influenced by the number and/or type of residues and/or spacing and/or phosphorylation of residues within a phosphate centre, for example by modification of the amino acids within the phosphate centres, (see later results in relation to the recombinant casein phosphopeptide wherein the inclusion of a number of Asp residues in place of rather fewer Glu residues in the phosphate centres of the recombinant CK2-S and CK2-SS peptides are considered to be a likely cause for increasing the size of nanoclusters prepared with these peptides compared to the nanocluster formed from the otherwise quite similar β-casein 1-25 phosphopeptide). In embodiments, the flanking sequences provided around phosphate centres can affect the area of the phosphate centre footprint by limiting the density of packing of peptides or protein in the shell. In this regard, it might be expected that a globular protein would be at a disadvantage compared to a short peptide or unfolded protein which might achieve a small footprint. Nevertheless, it can be envisaged that a globular domain, if it has an extended, flexible, linker sequence connecting it to a phosphate centre could be just as effective as a natively unfolded protein or short peptide.

It has been previously established that nanoclusters or structures containing nanoclusters formed from caseins or casein phosphopeptides, including whole casein, mixtures of β- and η-casein, β-casein 5P, digestion of whole casein with protease XIV from Streptomyces griseus, and papain type IV containing casein αₛ₁-casein 2P (f46-51), αₛ₁-casein 4P (f61-70), β-casein 4P (f11-21), αₛ₂-casein 3P (f5-12), αₛ₂-casein 4P (f49-61) and αₛ₂-casein 2P (f126-133), β-casein phosphopeptides, 4P (f1-25) or 4P (f2-25) or 5P (f1-42), cyanogen bromide cleavage fragments αₛ₁-casein 2P (f1-54) or αₛ₁-casein 6P (f61-123), tryptic phosphopeptides αₛ₁-casein 1P (f104-119) or αₛ₁-casein 2P (f43-58) or αₛ₁-casein 5P (f59-79) or αₛ₁-casein 7P (f43-79) can be made. The same techniques used to form these nanoclusters can be utilised in the present invention, which utilise alternative phophoprotein or phosphopeptide molecules. Examples of appropriate concentrations of calcium ions, phosphate ions and phosphoprotein or phosphopeptide molecules which provide for nanocluster forming solutions and which have an appropriate pH for the formation of calcium phosphate nanoclusters are provided herein and in U.S. Pat. No. 7,060,472, Holt C, Wahlgren N M, & Drakenberg T. (1996) Ability of a beta-casein phosphopeptide to modulate the precipitation of calcium phosphate by forming amorphous dicalcium phosphate nanoclusters. Biochem J., 314, 1035-1039; Holt C, Timmins P A, Errington N, & Leaver J. (1998) A core-shell model of calcium phosphate nanoclusters stabilized by beta-casein phosphopeptides, derived from sedimentation equilibrium and small-angle X-ray and neutron-scattering measurements. Eur. J. Biochem, 252, 73-78; Holt C, The milk salts and their interaction with Casein Advanced Dairy Chemistry, Vol 3: Lactose, Water, Salts and Vitamins (P. F. Fox, Ed.) Chapman and Hall, London pp 233-254 (1997). Little, E. M. and Holt, C. (2004) An equilibrium thermodynamic model of the sequestration of calcium phosphate by casein phosphopeptides. European Biophysics Journal 33, 435-447, Holt, C. (2004) An equilibrium thermodynamic model of the sequestration of calcium phosphate by casein micelles and its application to the calculation of the partition of salts in milk. European Biophysics Journal 33, 421-434.

In some embodiments, calcium phosphate nanoclusters have been made from complex salt mixtures, for example 37 mM Ca(NO₃), 6 mM Mg(NO₃)₂, 36 mM KNO₃, 25 mM KH₂PO₄, 5 mM K₂HPO₄, 26 mM KNO₃, 1.5 mM NaN₃ (as a preservative) or 30 mM Ca(NO₃)₂, 4 mM Mg(NO₃)₂, 10 mM tripotassium citrate, 20 mM KHPO₄, 26 mM KNO₃, 1.5 mM NaN₃ (as a preservative) which also include a phosphopeptide. Typically the solutions are initially pH 5.5 and the pH is raised to form the calcium phosphate nanoclusters. Suitably as described herein two methods may be used i) generating ammonia homogeneously in the solution to gently raise the pH by the catalytic hydrolysis of urea in the solution by urease or ii) by the simple mixing of a strong base and or a basic phosphate salt and an acidic solution of calcium, phosphate and phosphopeptide.

In some embodiments, the nanocluster forming solution has [Caₜ]/[PP] less than or equal to 3.1; [P_{i,t}]=(0.875±0.125), [PP]−1.67±0.25, where [PP] is the concentration of phosphopeptide in grams per litre, [P_{i,t}] is the total millimolar concentration of inorganic phosphate and [Caₜ] is the total millimolar calcium concentration.

In some embodiments, the nanoclusters can have an empirical formula in the range [Ca(HPO₄²⁻)_{0.4-1.0}(PO₄³⁻)(H₂Oₓ)]_{5.15}.[Ca₂₋₅₋SerP_{y}-peptide] where (y is greater than or equal to 3) and the sum of the charges of the ions within both square brackets is approximately zero and where Ca₂₋₅SerP_{y}-peptide is the calcium salt of a phosphorylated phosphopeptide molecule.

In embodiments a thermodynamically stable calcium phosphate nanocluster can be formed from a calcium phosphate nanocluster forming solution, wherein the calcium phosphate nanocluster forming solution is prepared by simple mixing of calcium ions, phosphate ions and phosphopeptide or phosphoprotein molecules with a concentration ratio of [Ca]/[Pₒ]≦3.1, where Ca is calcium and Pₒ is organic phosphorus, and wherein the pH of the calcium phosphate nanocluster forming solution is adjusted from an initial pH to a final pH by the simple mixing of the components of the calcium phosphate nanocluster forming solution, wherein said simple mixing of the components does not require urease to be present in the calcium phosphate nanocluster forming solution.

The term variant calcium binding phosphoprotein is meant a calcium binding phosphopeptide with greater than 70%, greater than 80%, greater than 90%, greater than 95%, or greater than 99% sequence homology to a calcium binding phosphoprotein or phosphopeptide, for example to a secretory/secreted calcium binding phosphoprotein/phosphopeptide or a member of the paralogous group of secretory/secreted calcium binding phosphoproteins.

Phosphopeptides 1 to 5 as listed below are not considered to be modified recombinant phosphopeptides of the invention.

(SEQ ID NO 5)
1) Glu-Met-Glu-Ala-Glu-Pse-Ile-Pse-Pse-Pse-Glu-
Glu-Ile-Val-Pro-Asn-Pse-Val-Gl u-Gln-Lys,
(SEQ ID NO 6)
2). Glu-Leu-Glu-Glu-Leu-Asn-Val-Pro-Gly-Glu-Ile-
Val-Glu-Pse-Leu-Pse-Pse-Pse-Gl u-Glu-Ser-Ile-Thr-
Arg,
(SEQ ID NO 7)
3). Asn-Thr-Met-Glu-His-Val-Pse-Pse-Pse-Glu-Glu-
Ser-Ile-Ile-Pse-Gln-Glu-Thr-Ty r-Lys,
(SEQ ID NO 8)
4). Asn-Ala-Asn-Glu-Glu-Glu-Tyr-Ser-Ile-Gly-Pse-
Pse-Pse-Glu-Glu-Pse-Ala-Glu-Va l-Ala-Thr-Glu-Glu-
Val-Lys,
and
(SEQ ID NO 9)
5). Glu-Gln-Leu-Pse-Pth-Pse-Glu-Glu-Asn-Ser-Lys,

(Pse is phosphoserine, Ser is serine, Pth is phosphothreonine, Thr is threonine, Glu is glutamate, Asp is aspartate, Ala is alanine, Asn is asparagine, Gln is glutamine, Gly is glycine, Arg is arginine, His is histidine, Ile is isoleucine, Leu is leucine, Lys is lysine, Met is methionine, Pro is proline, Tyr is tyrosine, and Val is valine).

Preferably, the nanocluster forming solution does not contain significant amounts (greater than 0.1 mM) of other calcium chelating agents, which are not phosphoproteins or phosphopeptides. Preferably, if a calcium chelating agent such as citrate is included there must be an increased amount of calcium allowed in the nanocluster forming solution.

The size of the nanocluster, as determined by small-angle X-ray or neutron scattering, depends of the radius of the hydrated amorphous calcium phosphate core, the thickness and structure of the peptide shell and the relative scattering length densities of the core and the shell. For a given peptide, the size of the core depends of the sequestering power of the peptide. In thermodynamic terms, the equilibrium size results from a balance of the negative free energy of sequestration by the peptide against the positive free energy of formation of the calcium phosphate core. The former is proportional to the core surface area, whereas the latter increases with the core volume.

In embodiments, a nanocluster of the invention can have a core radius greater than or equal to 3 nm, greater than or equal to 4 nm, greater than or equal to 5 nm, greater than or equal to 8 nm, greater than or equal to 10 nm, greater than or equal to 15 nm, or greater than or equal to 20 nm.

In embodiments a nanocluster of the invention, can have a core surface area per phosphate centre in the nanocluster less than 0.25 nm², less than 0.3 nm², less than 0.4 nm², less than 0.5 nm², less than 0.6 nm² or less than 1 nm².

Advantageously thermodynamically stable calcium phosphate nanoclusters of the invention may be i) biocompatible, because they are made from the normal non-toxic constituents of biological materials, and/or ii) immunologically silent through the use of peptides normally present in, for example, circulation in the blood or in an animal body, for example a human.

According to a second aspect of the present invention, there is provided a phosphopeptide for use in the method of the first aspect of the invention wherein the phosphopeptide comprises:
a calcium binding phosphopeptide or phosphoprotein, or a variant or a fragment thereof wherein the phosphopeptide or phosphoprotein does not include any caseins, or enzymatic digests of any casein.

According to a third aspect of the present invention, there is provided a recombinantly expressed phosphopeptide wherein said recombinant phosphopeptide:
i) includes a phosphate centre modified such that at least one of a phosphorylated residue and an acidic residue or combinations of these residues is increased within the phosphate centre such that the phosphate centre has increased calcium phosphate sequestering power, or
ii) is modified to include an increased number of discrete phosphate centres over a non-modified recombinantly expressed phosphopeptide, or
iii) is modified such that the modified recombinant phosphopeptide has increased calcium phosphate sequestering power over a non-modified version of the recombinantly expressed phosphopeptide, preferably by alteration of the number and/or type and/or phosphorylation of the amino acid residues or spacing of particular amino acid residues within a phosphate centre, or removing amino acid sequences which promote the conversion of amorphous calcium phosphate into a more crystalline phase such as apatite.

By forming nanoclusters using defined phosphopeptides of the invention opportunities are provided for the design of new and improved nanostructures.

Phosphopeptide—Secretory/Secreted Calcium binding phosphoprotein The phosphopeptides/phosphoproteins used to form a nanocluster can be selected according to the use of the nanocluster. Phosphopeptides or phosphoproteins can include a centre of phosphorylation and typically may comprise few or no hydrophobic regions. As will be appreciated by those of skill in the art, a nanocluster may be formed from a plurality of different types of phosphopeptides or phosphoproteins or combinations of both. In some biomedical applications, due to the immunogenicity of casein, it may be preferred to use non-casein phosphopeptides or phosphoproteins. In general a short phosphopeptide/phosphoprotein is preferred because less mass of phosphopeptide is required to provide a given molar concentration of phosphopeptide.

In embodiments the phosphopeptide may comprise a phosphate centre conjoined to a linker peptide of a length suitable to bridge the outer shell of a nanocluster. For example, considering a nanocluster of core radius r_{c} and shell thickness l formed from nₚ peptides, a single linker peptide of nᵣₑₛ residues attached to a phosphate centre on one end and a globular protein of radius r_{g} at the other end can be incorporated in the nanocluster if the linker peptide can span the shell. Thus the linker peptide needs to be at least as long as the longest flanking sequence in the peptides forming the shell. If more than one linker peptide is incorporated in each nanocluster then the steric hindrance of the globular protein attachment needs to be considered. In the limit where all nₚ shell molecules are attached to globular proteins, the minimum thickness of the shell is specified by the nanocluster surface area that is able to incorporate all nₚ globular proteins.

4π(r_{c}+l+r_{g})²≧4πnₚr_{g}²

The positive root of this quadratic equation in l is the minimum shell thickness, lₘᵢₙ, given by lₘᵢₙ≧(\sqrt{nₚ}−1)r_{g}−r_{c}. Thus the minimum number of residues in the linker peptide is obtained by dividing lₘᵢₙ by the length of a peptide bond in the fully extended conformation.

Useful globular proteins to attach to a nanocluster surface include antigenic proteins for use in vaccines and receptor ligands for drug delivery to targeted cells, but could include shorter functional sequences including linear epitopes, oligo-Arg or oligo-Lys sequences for the attachment of negatively charged molecules such as siRNA and oligo-Glu or oligo-Asp sequences for attachment to positively charged molecules or surfaces such as calcium oxalate.

In embodiments, the calcium binding phosphopeptide can be a secretory or secreted calcium binding phosphoprotein/phosphopeptide such as a member of the paralogous group of secretory calcium binding phosphoproteins or a variant or fragment of such proteins/peptides. In general terms, gene duplication to create members of a paralogous group permits the divergence of function. Moreover, adaptive mutations towards a novel function can compete with the original function so that the latter is lost. In view of this, it was by no means obvious to a person of skill in the art, in view of previous findings that calcium-sensitive caseins could form equilibrium nanoclusters, that other members of the group of secretory calcium binding phosphoproteins could share the ability to sequester calcium phosphate to form an equilibrium complex.

In embodiments of the invention, a phosphopeptide of the invention can be a member selected from the group consisting of
fetuin A (FETUA) (Swiss-Prot Accession No P02765) (SEQ ID NO 10),
proline-rich basic phosphoprotein 4 (PRB4) (Swiss-Prot Accession No P10163) (SEQ ID NO 11),
matrix Gla protein (MGP) (Swiss-Prot Accession No P08493) (SEQ ID NO 12),
secreted phosphoprotein 24 (SPP-24) (Swiss-Prot Accession No Q13103) (SEQ ID NO 13),
Riboflavin Binding Protein (Swiss-Prot Accession No P02752) (SEQ ID NO 14)
osteopontin (OPN) (Swiss-Prot Accession No P10451) (SEQ ID NO 15),
integrin binding sialophosphoprotein II (IBSP-II) (Swiss-Prot Accession No P21815) (SEQ ID NO 16),
matrix extracellular bone phosphoglycoprotein (MEPE) (Swiss-Prot Accession No Q9NQ76) (SEQ ID NO 17),
dentin matrix acidic phosphoprotein 1 (DMP1) (Swiss-Prot Accession No Q13316) (SEQ ID NO 18)
   or variants or fragments thereof and allowing for terminological variations in the trivial names given to these sequences.

Suitably, in embodiments of the phosphopeptide, all the phosphate sites in phosphate centres can be phosphorylated and none of the other phosphate sites in the peptides are phosphorylated such that sequestration is favoured over amorphous calcium phosphate (ACP) maturation. In embodiments a phosphopeptide of the invention can comprise Dentin Matrix acidic phosphoprotein 1 (DMP1), Fetuin A (FETUA), Matrix Gla Protein (MGP), Secreted phosphoprotein 24 (SPP-24), Osteopontin (OPN) or integrin-binding sialophosphoprotein (IBSPII) isoforms with at least one phosphate centre including at least three phosphorylated residues. As the amino acid sequences of the complete proteins of DMP1, Fetuin A, MGP, SPP-24, OPN and IBSPII are considered likely to promote the conversion of amorphous calcium phosphate (ACP) into a more crystalline phase such as apatite, in preferred embodiments of such phosphopeptides, the phosphopeptide sequences of DMP1, Fetuin A, MGP, SSP-24, OPN and IBSPII, do not contain a cystatin domain D1, as found in Fetuin A and SPP-24, a cluster of 4 or more Gla residues, as found in MGP, oligo Glu sequences of 5 or, typically 8 or more consecutive Glu residues, as found in IBSPII, or long phosphorylated sequences containing 10 or more sites of phosphorylation, as found in the C-terminal half (after S-180 in rat) of DMP-1, or the C-terminal part (after residue K-149 in the cow) of OPN. Preferred phosphopeptide sequences may be formed by selective proteolytic cleavage of the parent protein using methods well known in the art. For example, cleavage of OPN from cow by plasmin at 149-K-K-150, cleavage of DMP1 from rat by an Asp-specific protease at 180-S-D-181 provides functional N-terminal peptides containing phosphate centres without the ACP to HA-transforming tendency of the C-terminal sequences. Alternatively, partial sequences of secreted calcium binding phosphoproteins/phosphopeptides can be produced by, for example, recombinant methods. In embodiments, the phosphopeptide can be osteopontin, or a variant or a fragment thereof modified such that it does not include a sequence likely to promote the conversion of amorphous calcium phosphate (ACP) into a more crystalline phase such as apatite. Preferably, the osteopontin amino acid sequence or a variant or a fragment thereof has substantially all sites of phosphorylation within phosphate centres. In embodiments, an osteopontin fragment of the invention may comprise SEQ ID NO 1 (osteopontin (OPN) 1-149). As used herein the term OPN 1-149 is used to describe a group of N-terminal phosphopeptides produced by plasmin catalysed hydrolysis. These include the sequence OPN 1-147, 1-149 and 1-150. In embodiments, an osteopontin fragment of the invention may consist of SEQ ID NO 1 (OPN 1-149). Suitably, the phosphopeptide used in the first aspect of the invention can be OPN 1-149 (SEQ ID NO 1).

It is considered by the inventors that formation of calcium phosphate nanoclusters by secreted phosphoprotein or peptides derived from secreted phosphoproteins may be effective in the body to some degree in maintaining the stability of milk and blood and some other biological fluids and the stability of the extracellular fluid in soft tissues. Likewise, the nanoclusters may be involved in controlling the degree of metastability and rate of phase separation of calcium phosphate in the extracellular matrix of mineralizing tissues or in reversing ectopic mineralization in soft tissues. The extraction, from living eukaryotic cells or tissues of an intrinsic intracellular protein in a defined state of phosphorylation may require the rigorous suppression of kinase and phosphatise activities. This can be difficult and time consuming. An alternative approach is to express the protein or polypeptide substrate in a defined state of phosphorylation using recombinant methods. This also allows for the modification or de novo synthesis of phosphopeptides.

### Phosphopeptide—Recombinant Phosphopeptide

A nanocluster with enhanced calcium phosphate sequestering power may include a recombinant phosphopeptide wherein said recombinant phosphopeptide has been modified to provide at least one of an increased number of phosphorylated residues within a phosphate centre, a phosphate centre with increased calcium phosphate sequestering power or an increased number of discrete phosphate centres. A recombinant phosphopeptide may be further modified at sequences distinct from the phosphate centres as desired, for example to reduce the length of the phosphopeptide. Phosphopeptides including a recombinant phosphate centre can be chemically synthesised de novo, or can be based on a known phosphopeptide, for example a secreted calcium binding phosphoprotein/phosphopeptide or a casein peptide into which mutations are introduced. Such mutations can increase the number of phosphorylated residues in known phosphate centres, and/or can increase the number of phosphate centres, and/or increase the calcium phosphate sequestering power. In embodiments a recombinant phosphopeptide can be based on bovine β casein. In embodiments a recombinant phosphopeptide can include enzyme digestion sites, for example tryptic cleavage sites. In embodiments of the invention a phosphopeptide can be a recombinant secretory calcium binding phosphoprotein which is a non-casein secreted phosphoprotein. In embodiments there is provided a recombinant casein phosphopeptide in which glutamate residues in the phosphate centre of the casein are substituted with aspartate residues. In such embodiments preferably a casein phosphate centre can be mutated to introduce Asp residues in preference to Glu residues wherein at least 3 Asp residues are provided to replace 3 Glu residues, more preferably at least 4 Asp residues are provided to replace 4 Glu residues.

In some embodiments, the recombinant phosphopeptide can comprise at least one of CK2-SS and CK2-S and CK2-S-6H (shown below). The CK2SS construct contains tandem repeats of the CK2-S sequence separated by tryptic cleavage sites.

| | | |
|---|---|---|
| CK2-SS | --ELEELNVPGADDpSpSpSpSDDDDDDD RINKK | (SEQ ID NO 2) |
| CK2-S | --ELEELNVPGADDpSpSpSDDDpSDDDDRINKK | (SEQ ID NO 3) |
| CK2-S-6H | MRELEELNVPGADDpSpSpSDDDpSDDDDRINKKIEDPNSpSSVDKLAAALEHHHHHH | (SEQ ID NO 4) |

Selective and exclusive phosphorylation of a polypeptide substrate can be achieved, to provide the phosphopeptide, by co-expressing a single recombinant kinase with the recombinant polypeptide substrate. For example, each of the phosphate centres in recombinant constructs may be phosphorylated by co-expression of the recombinant peptides with the catalytic subunit (CK2α) of the protein kinase—Casein Kinase 2. In relation to the recombinant phosphopeptides CK2-SS, CK2-S and CK2-S-6H, this produces a range of phosphoforms, most of which contain three or more phosphorylated residues in their phosphate centre. This provides a further aspect of the invention of a method of producing recombinantly expressed phosphopeptides comprising an average degree of phosphorylation at multiple sites, for example between about 61 to 83%, comprising providing two compatible cohabiting plasmids in, for example an E. coli host, the plasmids having high and low copy number, expressing a protein kinase and more abundantly a polypeptide substrate, respectively. E. coli is advantageous for use as a host cell due to its familiarity, ease of use and flexibility. Additionally, E. coli causes no background phosphorylation of the recombinantly expressed polypeptide by intrinsic protein kinases. In some embodiments the host strain of E. coli can be BL21 star [DE3 dualy transformed with a pET plasmid encoding the phosphorylation candidate protein/polypeptide and with pACYC Duet-1-hCK2α.]

As will be appreciated, any protein or peptide can be engineered to contain a suitable phosphate centre sequence and any globular protein can be extended at either its C- or N-terminus by a flexible linker sequence containing a phosphate centre capable of binding to the core surface of a nanocluster. If necessary, a recombinant peptide/protein may be unfolded by means of at least one of denaturing agent, disulphide bond reduction and pH adjustment. With care, the modified sequence can be incorporated in a range of thermodynamically stable nanoparticles formed from other known phosphopeptides or used to make novel phosphopeptides. In some embodiments, a phosphopeptide for use in the method comprises at least one phosphate centre flanked on one or each side by a flexible amino acid sequence. In embodiments, a phosphate centre may be flanked on one side by a predicted flexible sequence when the phosphate centre lies close to the N- or C-terminus. Flexible sequence may be determined using a suitable computer programme, for example PONDR® (http://www.pondr.com). A phosphopeptide containing an amino acid sequence likely to promote the conversion of amorphous calcium phosphate (ACP) into a more crystalline phase such as apatite may prevent the sequestration of calcium phosphate into equilibrium nanoclusters. In view of this, in preferred embodiments the phosphopeptide should not contain a sub-sequence that promotes the maturation of amorphous calcium phosphate into a more crystalline phase such as hydroxyapatite. In particular embodiments, the phosphopeptide contains few or no hydrophobic regions.

Whilst not wishing to be bound by theory, based on studies using the recombinant peptides (CK2-SS, CK2-S and CK2-S-6H), the studies conducted by the inventors in relation to OPN and tryptic phosphopeptides prepared from whole casein, it appears the length of the peptide or protein comprising the phosphate centre is not an important consideration. In addition, whilst the recombinant peptides provided as examples have similarity to bovine β-casein, the osteopontin plasmin (OPN) peptide has no significant sequence similarity to any casein sequence outside of the phosphate centres. Based on the present work, it would be expected suitable recombinant phosphoproteins/phosphopeptides based on OPN could be provided.

### Phosphate Centres

Phosphopeptides comprise a centre of phosphorylation. This can be a region of a peptide or protein containing three or more phosphorylated residues in a short sequence, for example around 5 to 9 residues in length, more preferably around 7 residues in length. In embodiments, the centre of phosphorylation can have at least three phosphorylated residues, preferably at least three phosphorylated residues close together in a multiply phosphorylated peptide, for example three phosphorylated residues in a series of six consecutive residues in a primary structure of a phosphopeptide or phosphoprotein molecule. The inventors have determined that competent phosphate centres (PCs) can be formed by phosphorylation or partial phosphorylation of a much broader range of primary structures than are found in caseins, for example than pS[I,L]pSpSpSEE (SEQ ID NO 223) (Tables 3 to 5 herein provide further examples of casein phosphate centres). In some embodiments the inventors have determined that phosphate centres contain three or more potential sites of phosphorylation, with typical phosphate centres containing four or five actual phosphorylated residues. In embodiments each phosphate centre can include a short acidic sequence including at least three phosphorylated residues in the primary structure of the phosphoprotein. Phosphate centres may contain a block of consecutive phosphorylation sites followed by the kinase primary recognition site of [S,T]EE, [S,T]ED, [S,T]DE or [S,T]DD, where residues in square brackets are alternatives, (Note [S,T]DD is not found in the caseins). Alternatively, a phosphate centre may include a minor pattern involving three or more repeats of a primary kinase recognition triplet [S,T]XE (Matrix Gla Protein (MGP)) or [S,T]DE (Osteopontin (OPN)).

According to PONDR® predictions, the positions of particular phosphate centre sequences in the subset of secreted phosphoproteins identified were invariably within a predicted flexible sequence in the secreted calcium binding phosphoproteins. These phosphoproteins can have two flanking sequences of low complexity or one such flanking sequence when the phosphate centre lies close to the N- or C-terminus of the phosphopeptide.

The largest number of sites of phosphorylation in a competent phosphate centre occurs in rat α_{S1}-casein with 9, 8 of the phosphorylation sites being consecutive and in two α_{S2}-casein sequences. Accordingly, and taking into account the pattern recognition methods discussed herein, a phosphate centre sequence preferably can have between three and nine sites of phosphorylation of which at least three are actually phosphorylated residues. Kinases responsible for phosphorylation of secreted phosphoproteins include, amongst others, the Golgi kinase and intranuclear and extracellular kinases with casein kinase 2-type specificity. Whilst kinase recognition sequences are not rigidly prescribed, the Golgi kinase and casein kinase 2 sites can fit the motif [S,T]X(m,n)[E,D,pS,pT], where X is any residue and X(m,n) denotes a variable sequence of length between m and n. Typically, for the Golgi kinase, m=n=1 and for casein kinase 2, m=n=2. In particular embodiments, such motifs may provide phosphate centres. Clustering of sites of phosphorylation and variability in the degree of phosphorylation are common among the secreted phosphoprotein and can be physiologically important.

Many of the identified phosphate centres contain at least two consecutive sites of phosphorylation. Examples include
i) the phosphate centre motif in eutherian β-caseins which is, with the exception of the horse sequence, pS[L,V]pS(2,3)EE (SEQ ID NO 213 and 214),
ii) PC-3 of the eutherian α_{S1}-caseins is pS[I,G,-]pS(3,7)EE (SEQ ID NO 215),
iii) PC-2 of α_{S2}-caseins is pS(3,5)EEpS(0,2) with the exceptions of the camel, rabbit B and mouse B sequences (SEQ ID NO 216),
iv) PC-1 of OPN is pS(1,2)[G,A]pSpSEE with the exception of the rat sequence (SEQ ID NO 217),
v) OPN PC-2 is pSpSEEpTDD with the exception of the rabbit and mouse sequence (SEQ ID NO 218),
vi) part of the conserved phosphate centre motif of IBSP-II is pSpSE(3,8) except for the chicken sequence, and
vii) the matrix extracellular bone phosphoglycoprotein (MEPE) phosphate centre is pSpSEpSpS[D,pS]pSGpSpSpSEpS(1,2) (SEQ ID NO 219).

All the known SPP-24 phosphate centres include the minimum motif pSpSEE (SEQ ID NO 220). The single known Proline-rich basic phosphoprotein 4 (PRB4) sequence has pSpSpSED (SEQ ID NO 221), but the chicken riboflavin binding protein (RBP) phosphate centre is not conserved.

Phosphate centres without consecutive sites of phosphorylation can also be formed by tandem repeats of a Golgi kinase recognition sequence [S,T]X[E,D,pS,pT]. Examples are found in Osteopontin phosphate centre 3 (OPN PC-3), Fetuin A and matrix Gla Protein. A consensus motif for this type of phosphate centre is (pSXE)₃ (SEQ ID NO 222). Where a protein contains one or more well conserved phosphate centres, the other phosphate centres may appear to show more variability or may be absent altogether. Examples include PC-1 and to a lesser extent PC-2 in α_{S1}-casein, PC-3 in α_{S2}-casein and all PCs other than PC-1 in DMP1. Typically, nanocluster-forming phosphoproteins include one or more phosphate centres and substantially flexible linker portions interposed between the phosphate centres. In embodiments the phosphate centre flanking sequences of calcium phosphate sequestering peptides have low residue diversity, few hydrophobic residues and no cysteine residues. In embodiments, the substantially flexible linkers in solution can be provided by unfolding the phosphoprotein. Unfolding may be provided for by means known to a person of skill in the art, including the use of denaturing agents, disulphide bond reduction or pH adjustment. Adjustment of pH away from the isoelectric pH, for example typically to very acidic or alkaline pH can often completely denature a globular protein and, in combination with the reduction or alkylation of disulphide bridges, will be sufficient in most cases to completely unfold the protein. Likewise, the addition of 6-M urea or guanidinium hydrochloride in combination with disulphide bond reduction or alkylation will be sufficient to unfold the great majority of proteins. The use of guanidinium hydrochloride is preferred over urea because fine pH adjustments by means of the urea/urease reaction are still possible in the presence of the former denaturation agent. Once unfolded, the conformation of proteins is to a large degree flexible.

According to a fourth aspect of the present invention there is provided a thermodynamically stable calcium phosphate nanocluster including a phosphopeptide of the second or third aspect of the invention.

According to a further aspect of the invention, there is provided a thermodynamically stable calcium phosphate nanocluster comprising a phosphoprotein or phosphopeptide wherein the phosphoprotein or phosphopeptide comprises at least one of:
a) a recombinantly expressed phosphopeptide wherein said recombinantly expressed phosphopeptide
   i) includes a phosphate centre modified such that at least one of a phosphorylated residue and an acidic residue or combinations of these residues is increased within the phosphate centre such that the phosphate centre has increased calcium phosphate sequestering power, or
   ii) is modified such that the modified recombinantly expressed phosphopeptide includes an increased number of discrete phosphate centres in comparison to a non-modified version of the recombinantly expressed phosphopeptide, or
   iii) is modified such the modified recombinant phosphopeptide has increased calcium phosphate sequestering power over a non-modified version of the recombinantly expressed phosphopeptide, or
b) a calcium binding phosphoprotein/phosphopeptide, or a variant or a fragment thereof wherein the phosphopeptide or phosphoprotein does not include an individual casein or a mixture of caseins or enzymatic digests of an individual casein or a mixture of caseins, or
c) a combination of a) and b).

In some embodiments the modified recombinant phosphopeptide can have increased calcium phosphate sequestering power over a non-modified version of the recombinantly expressed phosphopeptide through alteration of the number and/or type and/or phosphorylation of the amino acid residues or spacing of particular amino acid residues within a phosphate centre, modifying amino acid residues or the number of the amino acid residues flanking a phosphate centre, or removing amino acid sequences which promote the conversion of amorphous calcium phosphate into a more crystalline phase such as apatite.

In embodiments, the thermodynamically stable calcium phosphate nanocluster can be provided by the method of the first aspect of the invention.

In some embodiments the nanocluster can comprise phosphopeptides comprising SEQ ID NO 1 (OPN 1-149). Without wishing to be bound by theory, based on the modelling of the structure of the OPN containing nanocluster, the number of OPN 1-149 peptide chains on a nanocluster are believed by the inventors be in the range 2500 to 2700, in particular about 2618 peptide chains.

As discussed above, nanoclusters can be prepared under conditions that do not generate excess amorphous calcium phosphate at any time as this may mature to form a more crystalline phase before it can be sequestered to form the nanocluster solution. Methods of forming thermodynamically stable calcium phosphate nanoclusters are known in the art, see for example U.S. Pat. No. 7,060,472. Nanoclusters can be prepared by either a urea/urease method or by simple mixing and after 1-2 days of maturation achieve an equilibrium size which does not change on storage. The urea/urease method is preferred because it does not generate local excess concentrations during mixing and the rate of reaction can be controlled easily through the concentration of the enzyme. Notwithstanding this, the simple mixing method has been used successfully to generate casein phosphopeptide nanocluster solutions containing in excess of 300 mM Ca on a litre scale. Below mM peptide concentrations the nanoclusters are difficult to detect and characterise.

Suitably, in particular embodiments a nanocluster may include another component to provide the nanocluster with additional functions such as to direct the nanocluster to a cell type or to elicit an immunological response, for example OPN nanoclusters contain an RGD sequence that binds to integrin receptors. Integrin binding is a characteristic of a subgroup of the SCPPs. In particular embodiments, an OPN 1-149 nanocluster can be provided as part of an adjuvant. Such an adjuvant would contain antigen and might also include immunostimulating components or cell directing components. The use of OPN 1-149 or fetuin A is advantageous over the use of casein nanoclusters as, in contrast to casein, OPN or fetuin A provides for immunologically silent nanoclusters. Calcium phosphate nanoclusters and phosphopeptides of the present invention have applications in the prophylaxis, diagnosis and treatment of a wide range of disorders of mineral metabolism and in preventing the mineralization of prostheses, transplanted organs and kidney dialysis membranes. Further, they can be used to stabilise biofluids and/or biofluid substitutes.

Accordingly, a fifth aspect of the present invention provides the use of calcium phosphate nanoclusters according to the present invention, in prophylaxis, diagnosis and treatment of disorders of mineral metabolism. In particular embodiments, disorders can include osteoporosis, rickets, bone disease, calcification of the mammary gland, pathological calcification, demineralisation of mineralised tissue, for example teeth, and calcification of dental plaque. Ectopic calcification is a common complication in replacing heart valves and the leading cause of their replacement. It can also occur in the vasculature, increasing the risk of acute disease such as ischaemia, stroke and myocardial infarction. Ectopic calcification of tumours in soft tissues complicates their treatment by chemotherapy and reduces the effectiveness of the drug. As a result of mineral imbalance, such as through kidney failure, metabolic syndrome or diabetes, calcification of the visceral organs, including the lung, heart, aorta, kidney and stomach occurs frequently.

Suitably, the nanoclusters of the present invention may be used to control the stability of biological fluids or artificial fluids, for example synthetic blood serum, where there is a need to maintain supersaturation without the risk of precipitation, for example in blood dialysis. Accordingly a further aspect of the invention provides the use of a nanocluster containing solution to maintain the stability and degree of supersatuation of a natural or synthetic fluid. Preferably, the fluid can be a biological fluid such as blood, blood plasma, extracellular and lymphatic fluids, synovial fluid, cerebrospinal fluid, and saliva. The biofluid can be in contact with and supersaturated with respect to calcium phosphate and be required to maintain the mineralized state of tissues such as bone, teeth and osteoid. Preferably, a significant proportion of the total calcium and phosphate in the fluid can be provided in the form of nanoclusters to help buffer the ion concentrations and pH. Preferably, a fluid may have a free calcium concentration around the physiological value of blood serum, in particular about 1.25 mM, and an isotonic osmolarity of 280-310 mM. Suitably the nanoclusters can include a phosphoprotein or phosphopeptide normally present in plasma, such as OPN, fetuin A, SPP-24 or matrix Gla protein, so that the nanoclusters have a low immunogenicity. In particular embodiments the phosphopeptide can be OPN 1-149 or a variant thereof.

According to a further aspect of the present invention there is provided a formulation wherein some proportion of the total calcium is present in the form of calcium phosphate nanoclusters and an excess of the sequestering phosphopeptide is present. It is a particular advantage of the technology that an artificial biofluid containing nanoclusters of the invention can contain physiological concentrations of calcium and phosphate and still be terminally sterilized without producing a precipitate of calcium phosphate. In embodiments, a formulation comprising nanoclusters of the invention can include an optimum level of phosphate and a phosphopeptide or phosphoprotein containing at least one phosphate centre, for example, the solution can have a pH of 7.3-7.5, an osmolarity of 280-310, and an oncotic pressure of 20-30 mm Hg. Osmolarity is determined primarily by the electrolytes, as well as by the oncotic agent, the phosphopeptide and optionally glucose (preferably 0-125 mM). Agents, such as dextran (0-100 gm/l) and polyethylene glycol (0-25 gm/l) may be added to give the required oncotic pressure. Optionally, anti-oxidant or free radical scavengers, such as mannitol (0-20 gm/l), glutathione (0-4 gm/l), ascorbic acid (0-0.3 gm/l) and vitamin E (0-100 IU/l) may be provided. In embodiments, the phosphopeptide can be present in the range 0.5-2.0 mM, total Ca⁺⁺ in an amount ranging from about 0.5 to 4.0 mM; total Cl⁻ in an amount ranging from 70 to 160 mM; total Mg⁺⁺ in an amount ranging from 0 to 10 mM; total K⁺ in an amount ranging from 0 to 5 mM; total phosphate in an amount ranging from 5-15 mM and, optionally, a simple hexose sugar from 2 to 50 mM. The solution may be terminally heat sterilized. NaHCO₃ may be added as a commercially-available sterile 1 M solution to the sterilized formulation immediately before use. Generally, 5 ml of a 1 M NaHCO₃ solution can be added per litre, but more may be added.

The solution can include concentrations of calcium, sodium and magnesium ions which are within the range of normal physiological concentrations of said ions in plasma. In general, the desired concentration of these ions can be obtained from dissolved chloride and phosphate salts of sodium which are also in solution. In embodiments the sodium ion concentration is preferably in a range from 70 mM to about 160 mM, and preferably in a range of about 130 to 150 mM. The concentration of total calcium can be in a range of about 0.5 mM to 4.0 mM, and preferably in a range of about 2.0 mM to 2.5 mM. The concentration of total magnesium can be in a range of 0 to 10 mM, and preferably in a range of about 0.3 mM to 0.45 mM. The concentration of free calcium ions can be in the range of 0.5 to 2 mM and is preferably 1.25 mM, being the normal concentration in normal plasma. The free magnesium ion concentration can be in the range 0.2 to 1.0 mM and is preferably 0.6 mM being the concentration in normal plasma. The concentration of chloride ion can be in the range of 70 mM to 160 mM, preferably in the range of 110-125 mM Cl⁻. The solution can also include an amount of simple hexose sugar such as glucose, fructose and galactose, of which glucose is preferred. In a preferred embodiment of the invention nutritive hexose sugars can be used and a mixture of sugars can be used. In general, the concentration of sugar can be in a range between 2 mM and 10 mM with concentration of glucose of 5 mM being preferred. At times, it may be desirable to increase the concentration of hexose sugar in order to lower fluid retention in the tissues of a subject. Thus the range of hexose sugar may be expanded up to about 50 mM if necessary to prevent or limit oedema in the subject under treatment. The oncotic agent can be comprised of molecules whose size is sufficient to prevent their loss from the circulation by traversing the fenestrations of the capillary bed into the interstitial spaces of the tissues of the body. As a group, oncotic agents are exemplified by blood plasma expanders. Human serum albumin is a blood plasma protein used to expand plasma volume. Also known are polysaccharides, generally characterized as glucan polymers which are used as blood plasma expanders. In general, it is preferred that the polysaccharide is non-antigenic.

In particular embodiments, nanoclusters of the invention may be used to prevent mineralization of prostheses, transplanted organs and kidney dialysis membranes.

According to further aspects of the present invention, there is provided the use of a phosphopeptide and/or of a nanocluster(s) according to the invention:
i) as a high surface area support medium for catalysts,
ii) as a carrier of a receptor or receptor ligand,
iii) as a vaccine adjuvant,
iv) to enable the targeted delivery of drugs or nutrients, or
v) to elicit an enhanced and selective immunological response to an antigen
vi) to provide for calcium containing food or beverage.

Nanoclusters of the invention, formed from immunologically silent competent phosphopeptides derived from normal blood proteins such as OPN, fetuin A, SPP-24 or matrix Gla protein, for example, OPN 1-149, are advantageous over casein nanoclusters, as casein is not a normal blood protein and hence could elicit an immune response when used as an adjuvant. Immunologically silent nanoclusters, which are not recognised by the immune system as non-self and thus do not trigger an immune response, can have particular applications in drug delivery, as vaccine adjuvants and in bone cement applications, among others. Moreover, tailoring of phosphopeptides of the invention for use in forming nanoclusters of the invention, for example using the methods of the invention, allow adaptation of the properties of the nanoclusters for the particular end use of the nanocluster, for example, the pH/dissociation profile of the nanocluster may be altered, and/or moieties may be added to the phosphopeptides/nanoclusters to allow for chemical coupling of targeting molecules, drugs, for example cytotoxic drugs, immunostimulatory molecules or the like to the outside of the nanoclusters.

In an aspect of the invention, phosphopeptides and/or nanoclusters of the invention may be provided in a composition for provision to the body. For example phosphopeptides may be provided as a component of a mouthwash, dentifrice, toothpaste, gum, gel or another suitable solid phase for application to teeth to inhibit calcification of dental plaque. In addition, such compositions may include abrasive agents, polishing materials and/or antibacterial agents as would be known in the art. Nanoclusters may be provided in nutritional products, nutraceuticals and pharmacological preparations, to provide delivery of macro and trace minerals and other dietary supplements. Alternatively, phosphopeptides and/or nanoclusters of the invention may be provided in materials suitable for the manufacture of coatings for medical prosthesis including, but not limited to, catheters and heart valves. Calcium phosphate nanoclusters prepared using the methods or phosphopeptides of the present invention may be provided in compositions further comprising a dispersing agent and/or a preservative, for example a non-ionic detergent n-octylglucoside or a cationic detergent sodium dodecyl sulphate or bacteriostatic additives such as sodium azide or thymerosal. In addition, or alternatively, the resulting nanoclusters or nanocluster compositions may be pasteurised or sterilised and/or freeze dried. A freeze-dried nanocluster powder prepared from OPN 1-149 was found to dissolve virtually instantly in the original volume of water to form a clear solution without changing the size of the nanocluster. The maturation of the nanocluster solutions prepared by simple mixing could involve the reversal of an initial precipitate of amorphous calcium phosphate through the formation of more thermodynamically stable nanoclusters. Nanoclusters of the present invention can also have utility for cellular drug delivery or nucleic acid, for example RNAi, delivery vehicles.

### DEFINITIONS

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined below are more fully described by reference to the specification as a whole.

By the term fragment is meant a portion of a phosphopeptide or phosphoprotein specifically referred to herein which can suitably be used to form a nanocluster, for example a fragment which retains suitable phosphate centres. Preferably such fragments do not include an amino acid subsequence which promotes the formation of hydroxyapatite from amorphous calcium phosphate. Fragments may be generated by any suitable way known to those of skill in the art. Suitable ways of generating fragments include, but are not limited to, recombinant expression of a fragment from encoding DNA, via chemical synthesis or fragmentation of cognate proteins, either chemically or enzymatically. Fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers.

As used herein, the term “recombinant” refers to a peptide created using molecular biological manipulations, including but not limited to, expression of a peptide by a recombinant expression vector.

As used herein, “protein” refers to any composition comprised of amino acids and recognized as a protein by those of skill in the art. The terms “protein,” “peptide” and polypeptide are used interchangeably herein and wherein a peptide is a portion of a protein, those of skill in the art understand the use of the term in context. Likewise the terms “phosphoprotein”, “phosphopeptide” and “phosphopolypeptide” are used interchangeably herein and wherein a phosphopeptide or phosphopolypeptide are portions of a phosphoprotein, those of skill in the art understand the use of the term in context.

Variants of phosphopeptides of the present invention include functionally similar proteins which are considered to be “related proteins”. In some embodiments, these related proteins are derived from a different genus and/or species, including differences between classes of organisms (e.g., a bacterial protein and a fungal protein). In additional embodiments, related proteins are provided from the same species. The term variant also includes specific peptides specifically referenced herein, for example one of SEQ ID NOs 1 to 4 and 10 to 18 which have been modified to provide for addition of one or more amino acids to either or both of the C- and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence, whilst retaining function as a phosphoprotein capable of forming nanoclusters. The preparation of such a variant is preferably achieved by modifying a DNA sequence which encodes for a phosphoprotein as specifically referenced herein, transformation of that DNA sequence into a suitable host, and expression of the modified DNA sequence to form the variant. As will be understood, variant phosphoproteins/phosphopeptides can differ from a parent phosphoprotein/phosphopeptide (e.g. a peptide specifically referenced herein) and from one another by a small number of amino acid residues. The number of differing amino acid residues may be one or more, preferably 1, 2, 3, 4, 5, 10, 15, 20, 30, 40, 50, or more amino acid residues. In one preferred embodiment, the number of different amino acids between variants is between 1 and 10. In particularly preferred embodiments, related proteins and particularly variant proteins comprise at least 50%, 60%, 65%, 70%, 75%, 80%, 85%, 90%, 95%, 97%, 98%, or 99% amino acid sequence identity to a peptide specifically referenced herein. Additionally, a related protein or a variant protein as used herein, refers to a protein that differs from a member selected from the group consisting of fetuin A (Swiss-Prot Accession No P02765), proline-rich basic phosphoprotein 4 (Swiss-Prot Accession No P10163), matrix Gla protein (Swiss-Prot Accession No P08493), secreted phosphoprotein 24 (Swiss-Prot Accession No Q13103), riboflavin binding protein (Swiss-Prot Accession No P02752), osteopontin (Swiss-Prot Accession No P10451), integrin binding sialophosphoprotein (Swiss-Prot Accession No P21815), matrix extracellular bone phosphoglycoprotein (Swiss-Prot Accession No Q9NQ76), or dentin matrix acidic phosphoprotein 1 (Swiss-Prot Accession No Q13316) in a number of regions, for example at a phosphate centre. For example, in some embodiments, variant proteins have 1, 2, 3, 4, 5, or 10 corresponding regions that differ from a member selected from the group consisting of fetuin A (Swiss-Prot Accession No P02765), proline-rich basic phosphoprotein 4 (Swiss-Prot Accession No P10163), matrix Gla protein (Swiss-Prot Accession No P08493), secreted phosphoprotein 24 (Swiss-Prot Accession No Q13103), riboflavin (Swiss-Prot Accession No P02752), osteopontin (Swiss-Prot Accession No P10451), integrin binding sialophosphoprotein (Swiss-Prot Accession No P21815), matrix extracellular bone phosphoglycoprotein (Swiss-Prot Accession No Q9NQ76), or dentin matrix acidic phosphoprotein 1 (Swiss-Prot Accession No Q13316). Suitably, if the regions include a phosphate centre, the phosphate centre retains the ability to be phosphorylated. Residues which may be considered for substitution, insertion or deletion to form a variant protein can include conserved residues or others which are not conserved. In the case of residues which are not conserved, the replacement of one or more amino acids can be limited to substitutions which produce a variant which has an amino acid sequence that does not correspond to one found in nature (naturally occurring sequences). In the case of conserved residues, such replacements should not result in a naturally-occurring sequence. As will be appreciated, where a variant includes modification of amino acid sequence at a phosphate centre, the phosphate centre should retain its ability to be phosphorylated and may have increased phosphorylation. In some embodiments, the term variant can refer to a protein/peptide that provides similar function, tertiary structure, and/or conserved residues as a member selected from the group consisting of fetuin A (Swiss-Prot Accession No P02765), proline-rich basic phosphoprotein 4 (Swiss-Prot Accession No P10163), matrix Gla protein (Swiss-Prot Accession No P08493), secreted phosphoprotein 24 (Swiss-Prot Accession No Q13103), riboflavin binding protein (Swiss-Prot Accession No P02752), osteopontin (Swiss-Prot Accession No P10451), integrin binding sialophosphoprotein (Swiss-Prot Accession No P21815), matrix extracellular bone phosphoglycoprotein (Swiss-Prot Accession No Q9NQ76), or dentin matrix acidic phosphoprotein 1 (Swiss-Prot Accession No Q13316). In specific embodiments, a variant refers to a protein/peptide that provides similar function, tertiary structure, is substantially identical and/or is a homologue of a member selected from the group consisting of fetuin A (Swiss-Prot Accession No P02765), proline-rich basic phosphoprotein 4 (Swiss-Prot Accession No P10163), matrix Gla protein (Swiss-Prot Accession No P08493), secreted phosphoprotein 24 (Swiss-Prot Accession No Q13103), riboflavin binding protein (Swiss-Prot Accession No P02752), osteopontin (Swiss-Prot Accession No P10451), integrin binding sialophosphoprotein (Swiss-Prot Accession No P21815), matrix extracellular bone phosphoglycoprotein (Swiss-Prot Accession No Q9NQ76), or dentin matrix acidic phosphoprotein 1 (Swiss-Prot Accession No Q13316). In particular embodiments, a variant can be a homologue of a member selected from the group consisting of fetuin A (Swiss-Prot Accession No P02765), proline-rich basic phosphoprotein 4 (Swiss-Prot Accession No P10163), matrix Gla protein (Swiss-Prot Accession No P08493), secreted phosphoprotein 24 (Swiss-Prot Accession No Q13103), riboflavin binding protein (Swiss-Prot Accession No P02752), osteopontin (Swiss-Prot Accession No P10451), integrin binding sialophosphoprotein (Swiss-Prot Accession No P21815), matrix extracellular bone phosphoglycoprotein (Swiss-Prot Accession No Q9NQ76), or dentin matrix acidic phosphoprotein 1 (Swiss-Prot Accession No Q13316). A homologue can be a protein/peptide from a different, but usually related species, which corresponds and encompasses proteins that are separated by speciation (i.e., the development of new species) (e.g., orthologous genes), as well as genes that have been separated by genetic duplication (e.g., paralogous genes). As used herein, “orthologue” and “orthologous genes” refer to genes in different species that have evolved from a common ancestral gene (i.e., a homologous gene) by speciation. Typically, orthologues retain the same function during the course of evolution. Identification of orthologues finds use in the reliable prediction of gene function in newly sequenced genomes. As used herein, “paralogue” and “paralogous genes” refer to genes that are related by duplication within a genome. While orthologues retain the same function through the course of evolution, paralogues evolve new functions, even though some functions are often related to the original one. In particular embodiments a homologue is an orthologue. One indication that two proteins are substantially identical is that the first protein is immunologically cross-reactive with the second protein. Typically, proteins that differ by conservative amino acid substitutions are immunologically cross-reactive. Thus, a protein can be considered substantially identical to a second protein, for example, where the two peptides differ only by a conservative substitution.

The degree of homology between sequences may be determined using any suitable method known in the art (See, for example, Smith and Waterman, Adv. Appl. Math., 2:482 [1981]; Needleman and Wunsch, J. Mol. Biol, 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]), or BLAST, described by Altschul et ah, (Altschul et ah, J. Mol. Biol., 215:403-410, [1990]. As used herein, “percent (%) nucleic acid sequence identity” is defined as the percentage of nucleotide residues in a candidate sequence that are identical with the nucleotide residues of the sequence. Another indication that two nucleic acid sequences which encode phosphopeptides are substantially identical is that the two molecules hybridize to each other under stringent conditions (e.g., within a range of medium to high stringency).

As used herein, the term “hybridization” refers to the process by which a strand of nucleic acid encoding a phosphopeptide joins with a complementary strand through base pairing, as known in the art. “Hybridization conditions” refer to the conditions under which hybridization reactions are conducted and these conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm−5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions can be based upon the salt or ionic strength conditions of hybridization and/or one or more stringency washes. For example, 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, one will typically desire to employ relatively stringent conditions to form the hybrids (e.g., relatively low salt and/or high temperature conditions are used). Substantially similar and “substantially identical” in the context of at least two nucleic acids or polypeptides typically means that a polynucleotide or polypeptide comprises a sequence that has at least 60% identity, preferably at least 75% sequence identity, more preferably at least 80%, yet more preferably at least 90%, still more preferably 95%, most preferably 97%, sometimes as much as 98% and 99% sequence identity, compared to a reference (for example osteopontin OPN) peptide sequence.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers. As used herein, the singular “a”, “an” and “the” includes the plural reference unless the context clearly indicates otherwise.

Preferred features and embodiments of each aspect of the invention are as for each of the other aspects mutatis mutandis unless context demands otherwise.

Embodiments of the invention will now be described, with reference to the following examples which are provided for the purpose of illustration and are not intended to be construed as being limiting on the present invention, wherein:

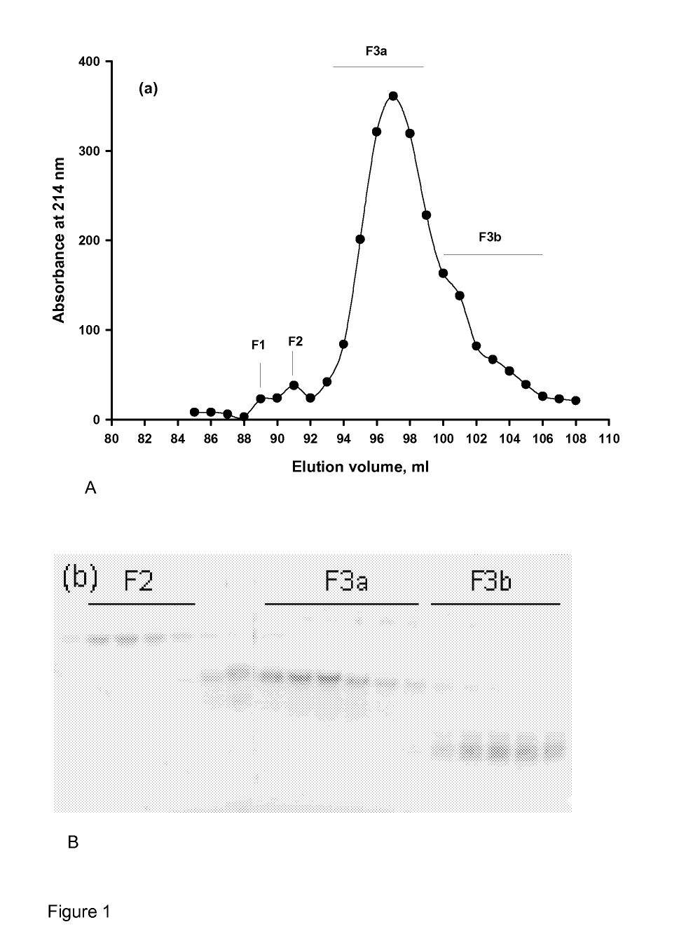

FIG. 1 (a) illustrates chromatographic separation of 10 mg of an OPN sample (OPNmix) on Superdex 75, with two small peaks, F1 and F2 being detected on the leading edge of a main peak; (b) SDS mops gel electrophoresis of individual tubes collected from the chromatographic separation shown in FIG. 1b. —no material penetrated the gel from peak F1 and subsequent fractions contained progressively smaller proteins or peptides, tubes were pooled to create the fractions F2, F3a and F3b, with the F2 fraction providing a single band with an apparent molecular weight of 50-55,000 Da and the F3 samples providing two main groups of bands which were pooled as F3a and F3b. The wt % of F2, F3a and F3b from the recovered weights were 10.2, 56.7 and 33.1, respectively. MALDI-MS measurements and calculations showed that F2 was the full length form (OPN 1-262) and the F3a fraction was the N-terminal fragment OPN 1-149, probably formed by the action of the principal milk proteinase plasmin. Full length native OPN had an experimentally determined molecular weight of 33.9 kDa, of which 1.7 kDa was due to phosphate groups and approx. 2.9 kDa was due to O-bound glycans. For the N-terminal fragment in F3a, the corresponding figures were 19.8 kDa with contributions of 0.9 kDa from phosphate groups and 2.9 kDa from the glycans. Thus, the average degree of phosphorylation of the 28 potential phosphorylation sites in the whole protein was 79%. The N-terminal fragment analysis was consistent with an average of about 60-65% phosphorylation of the 16 sites in an N-terminal plasmin peptide. All the glycans (3-4 sites) were present in both.

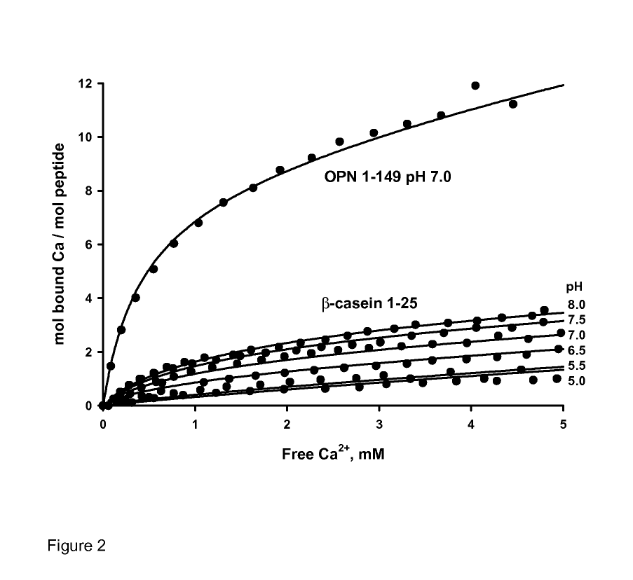

FIG. 2 shows the three Ca ion association constants, obtained by fitting the isotherms of β-casein 1-25, were: 3000, 400 and 30 M⁻¹. The isolated Pse residue had an effective pK of 6.0 and the cluster of three Pse residues ionised with a pK of 7.2. The OPN 1-149 isotherm was fitted by two Ca ion association constants of 3000 (dianionic phosphate) and 30 M⁻¹ and pKs of 6.4 and 5.0.

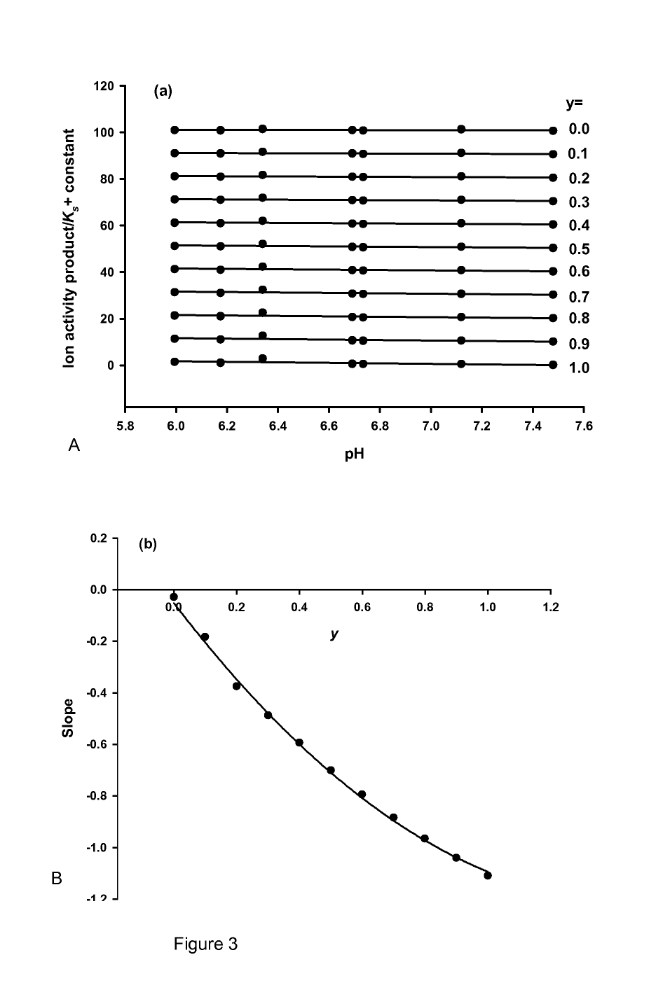

FIG. 3 illustrates the ion equilibria in ultrafiltrates of OPNmix nanocluster solutions. (a) Calculated ion activity products for y=0-1 in equation (9). (b) Slope of the ion activity product versus y.

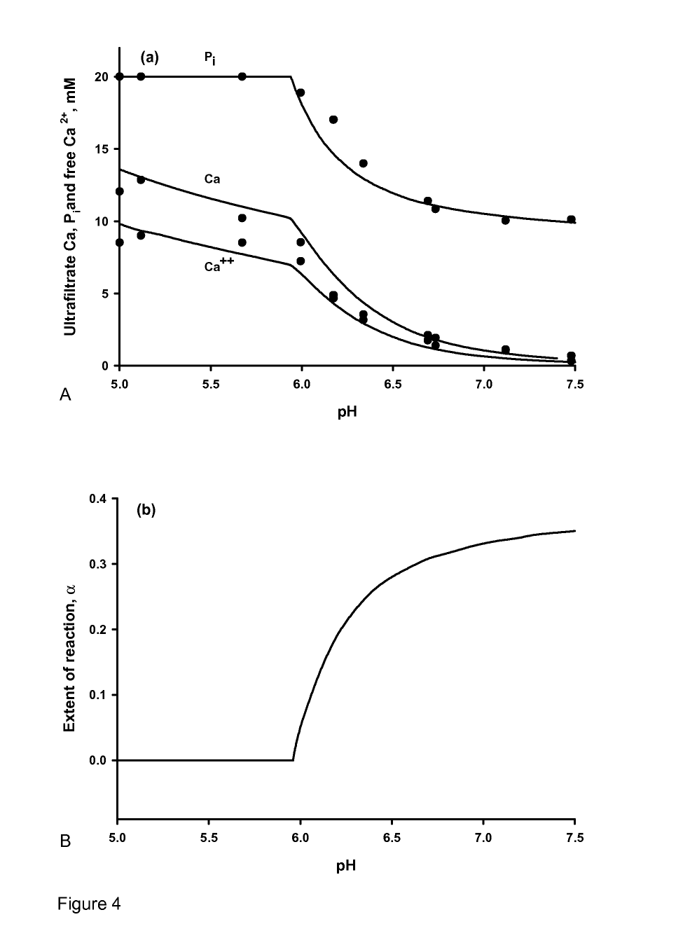

FIG. 4 shows calculated properties of OPNmix nanocluster solutions. (a) Comparison of calculated ultrafiltrate concentrations of Pᵢ, Ca and free Ca²⁺ with experimental values shown as symbols. (b) Calculated fraction of reacted PCs.

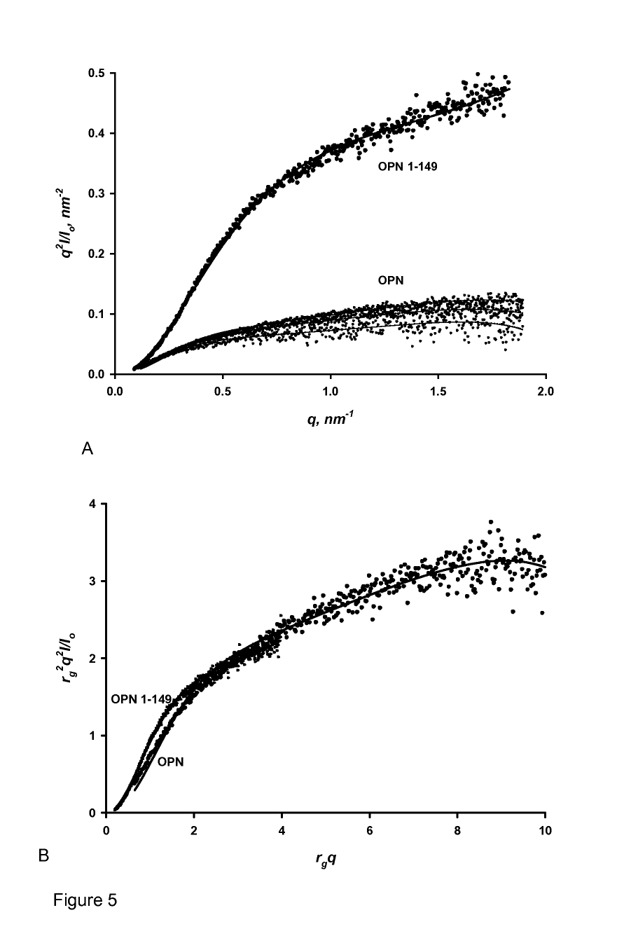

FIG. 5 shows Kratky plots of the SAXS of OPN in 20 mM Pᵢ buffer, pH 7.0, ionic strength 80 mM and of OPN 1-149 measured in the Calcium phosphate dilution buffer used in the nanocluster experiments. Fitted curves are from the worm-like chain model. (a) Effect of concentration on the q² weighted normalised SAXS of OPN at 5, 10 and 15 mg ml⁻¹ and OPN 1-149 at 10 mg ml⁻¹. (b) q² weighted SAXS of OPN and OPN 1-149 at 10 mg ml⁻¹, each axis is scaled by the root mean square radius of gyration determined by the fitting procedure.

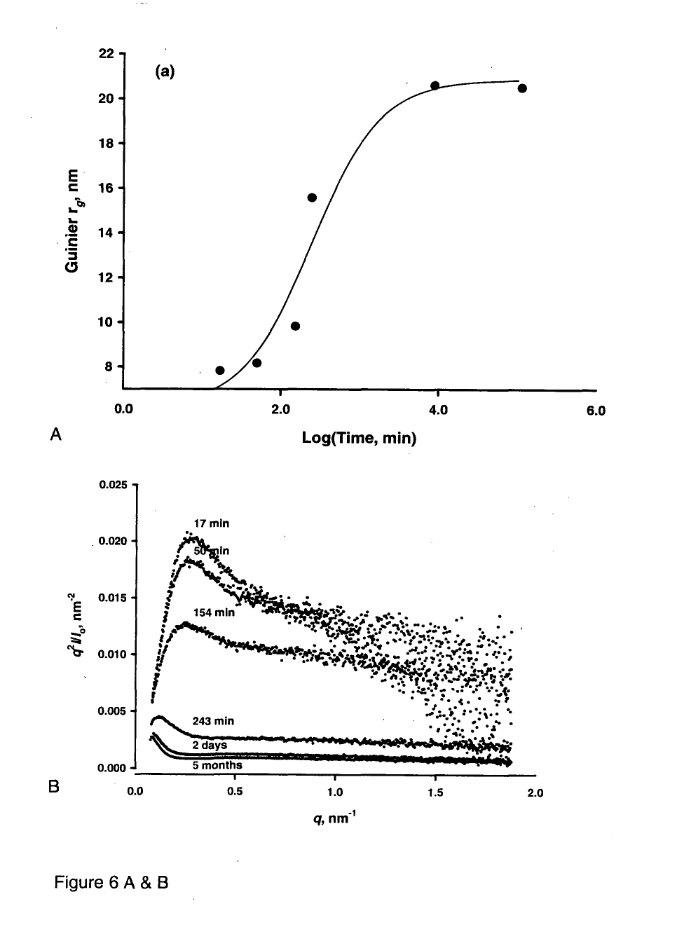

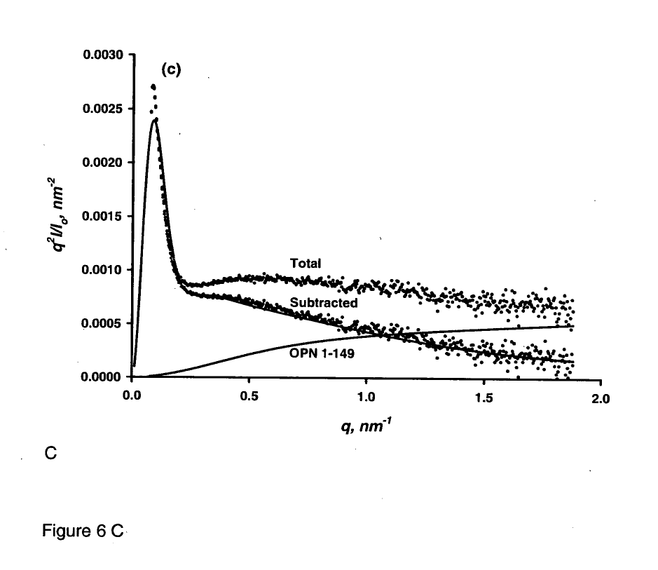

FIG. 6 shows the results of a study by SAXS of the maturation of nanoclusters made with OPN 1-149 by the urease method. (a) Effect of time on the radius of gyration determined by the Guinier method. (b) Normalised, q²-weighted SAXS of the nanoclusters diluted to 5 mg ml⁻¹ after the given times. (c) Model of the scattering of the matured nanocluster solution as a mixture of scattering from Gaussian copolymer micelle-like nanoclusters and free peptide. The scattering of the nanoclusters was obtained by subtracting the scattering of the free peptide from the total scattering. Model calculations used the parameters b=0.07 nm, A_{core}=0.25 nm², rₒ=12.5 nm, β=0.35.

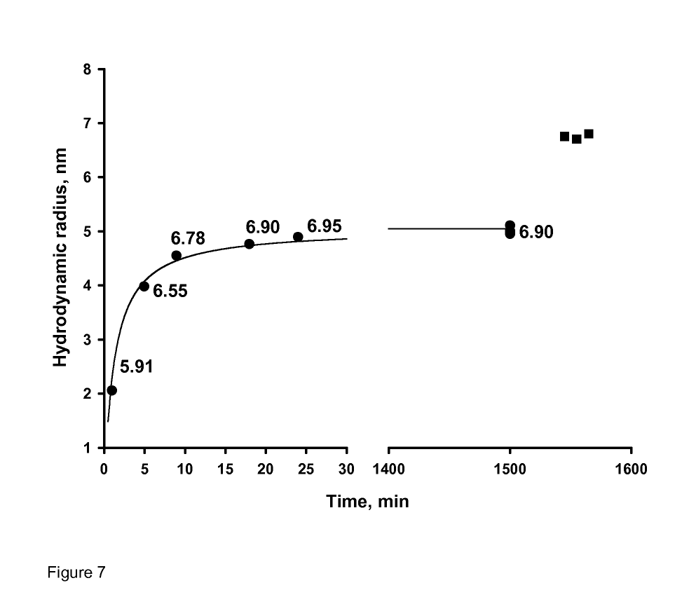

FIG. 7 illustrates the effect of pH (time) on the hydrodynamic radius of β-casein 1-25 nanoclusters () and the equilibrium size after 1 day of CPP nanoclusters (▪).

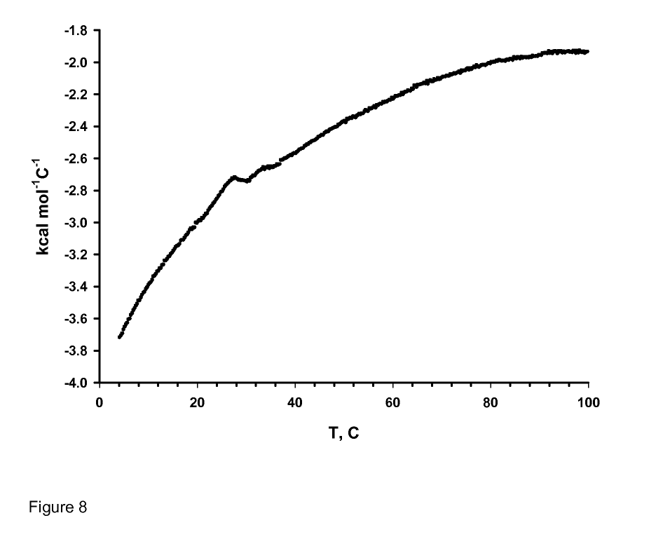

FIG. 8 illustrates a normalised DSC thermogram of OPN 1-149 at pH 7.0.

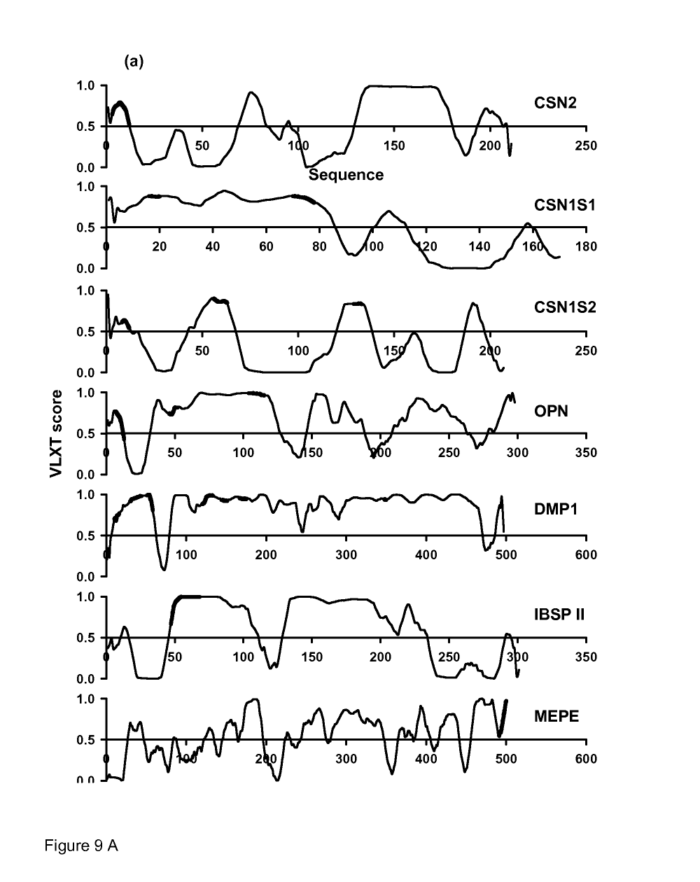

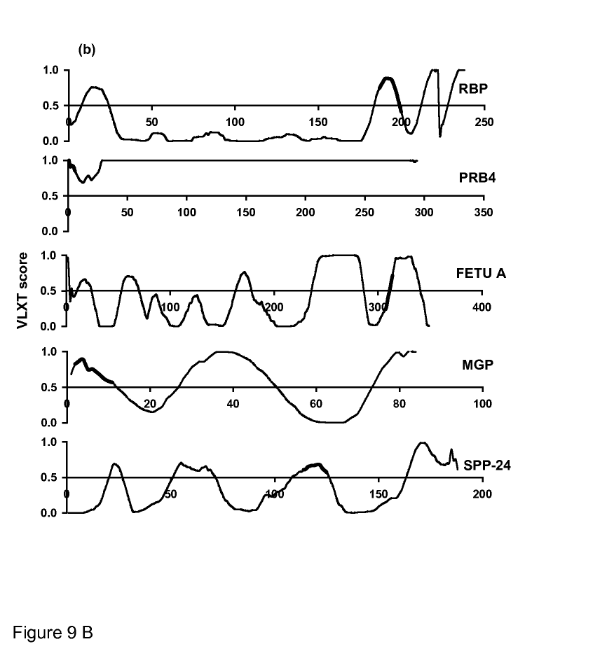

FIG. 9 illustrates prediction of disorder in secreted phosphoproteins having known or potential phosphate centre sequences with the position of known or predicted phosphates are shown in the heavier line (a) SCPPs, (b) non-SCPPs.

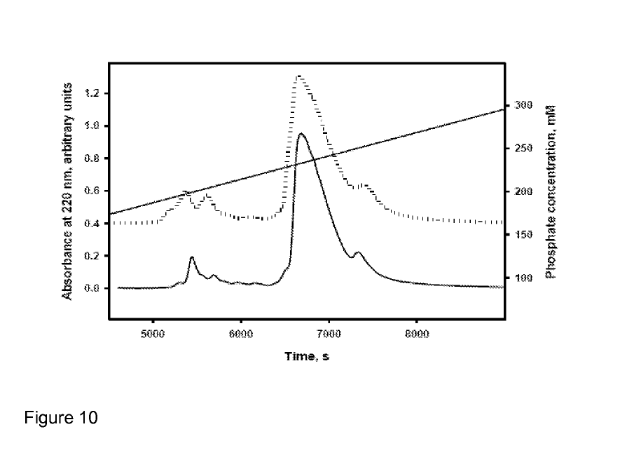

FIG. 10 illustrates a hydroxyapatite chromatography trace of the recombinant phosphopeptides CK2-S-6H and CK2-S with two representative absorbance profiles (220 nm) shown—continuous line, CK2-S; dashed line, CK2-S-6H—superimposed on the calculated linear phosphate concentration gradient during part of stage 2 of the method.

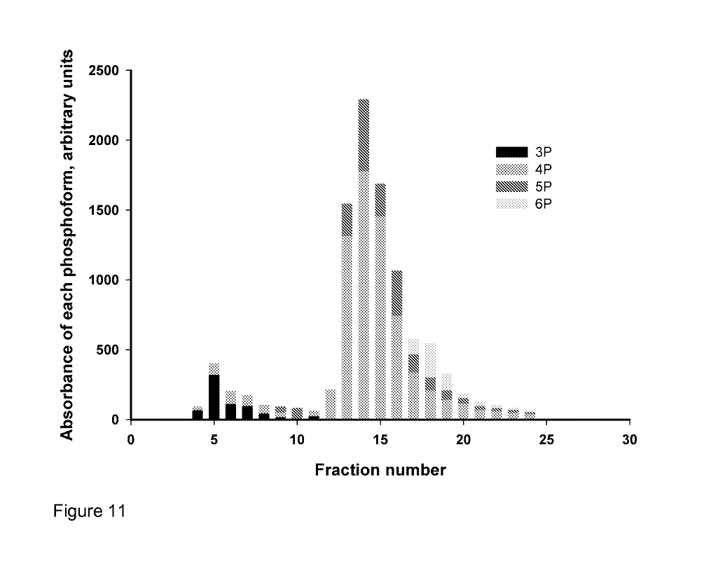

FIG. 11 illustrates the distribution of different phosphoforms of CK2-S-6H across its hydroxyapatite chromatography profile wherein fractions (300 s) were collected during a separation by hydroxyapatite chromatography of CK2-S-6H (see dashed-line chromatogram in FIG. 10) and each fraction was subjected to tandem nano-LC/MS analysis for quantitation of the different degrees of phosphorylation (from 3 to 6 mol P/mol of peptide) present. The greyscale bar-chart shows the mol fractions multiplied by the average absorbance of the fraction with the 4P phosphoform in particular occurring in both the main peak and the leading minor peaks.

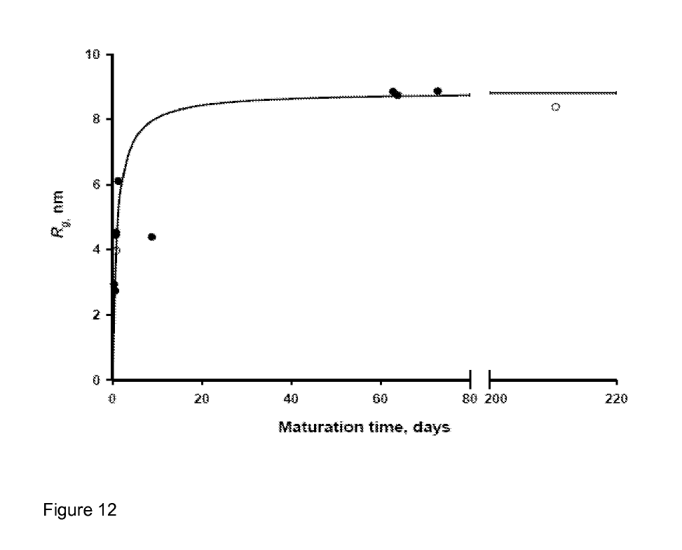

FIG. 12 illustrates the effect of maturation time on the radius of gyration (R_{g}) of nanoclusters prepared with the phosphopeptides CK2-S and CK2-SS with filled circles being for 4 different preparations of CK2-S and open circles being for a sample from a single batch of CK2-SS measured on two separate occasions—replicate and repeat measurements were extended over 3 beamtime allocation periods and the single line fitting the observations was calculated by a non-linear unweighted least squares regression of the equation R_{g,t}=R_{g,∞}/(1+t_{1/2}/t), where t_{1/2} is the half time for maturation, R_{g,t} is the radius of gyration at time t and R_{g,∞} its fully matured value.

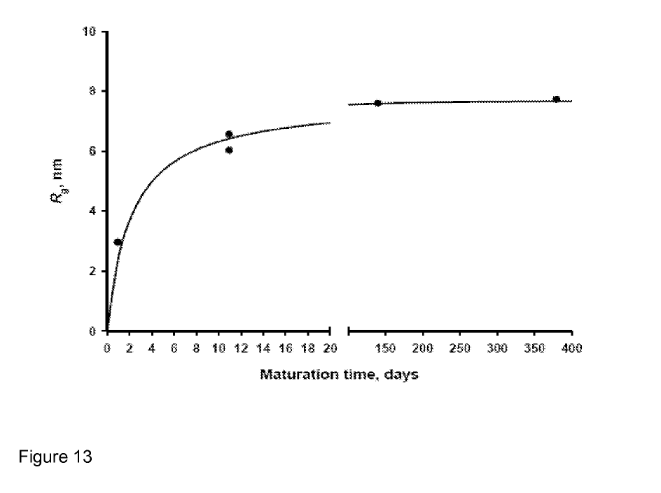

FIG. 13 illustrates the effect of maturation time on the radius of gyration (R_{g}) of nanoclusters prepared with the phosphopeptide CK2-S-6H wherein measurements were made on a single batch of the recombinant phosphopeptide and replicate and repeat measurements extended over 3 beamtime allocations—The single line fitting the observations was calculated as for FIG. 12.

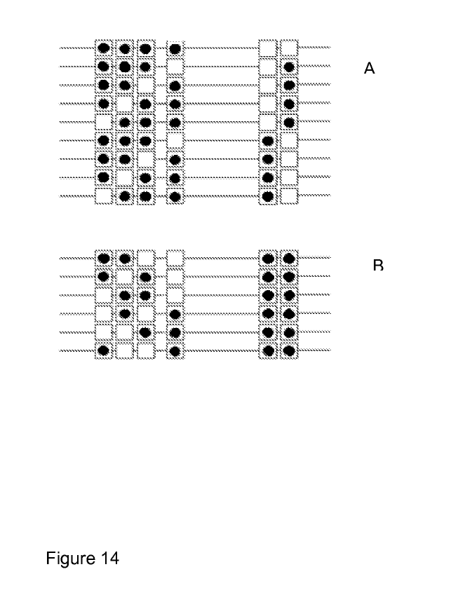

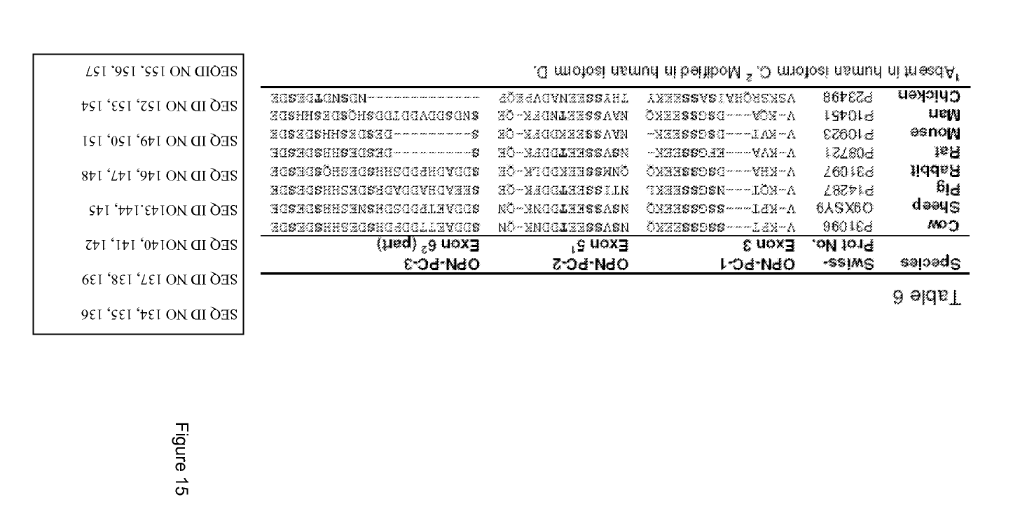

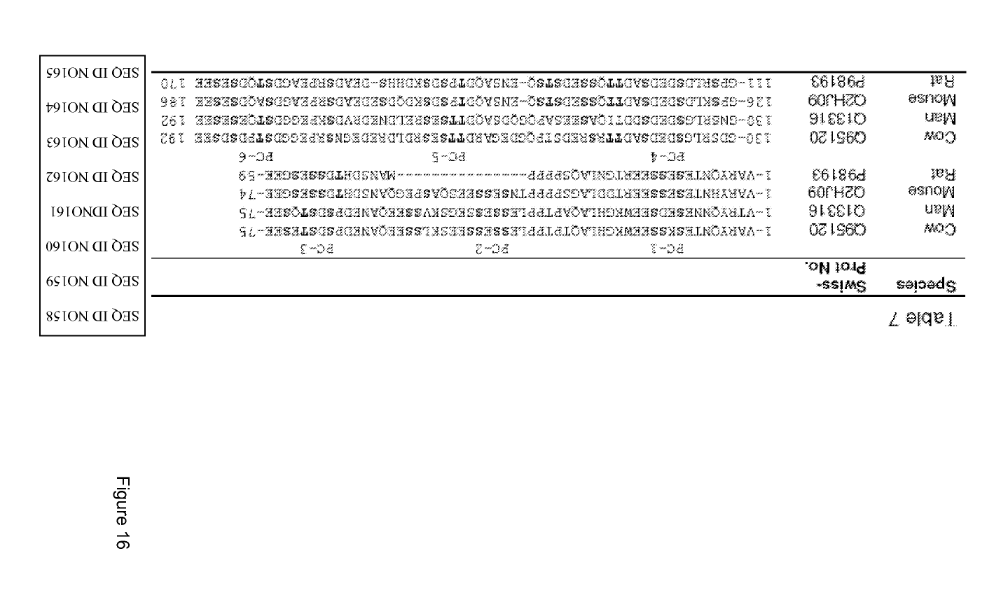

FIG. 14 illustrates the hypothesis for the existence of 4P phosphoforms of CK2-S-6H with (A) high- and (B) low-affinity for hydroxyapatite wherein a diagrammatic representation of the primary structure of CK2-S-6H with potential sites of phosphorylation shown as boxes is provided. Of the 6 sites, 4 are clustered together to form the phosphate centre sequence with the other two sites forming a second, minor, cluster closer to the C-terminus. A filled circle inside a box corresponds to a phosphorylated residue and an empty box to an unphosphorylated residue. Of the 15 positional isomers of the 4P phosphoforms, 9 have 3 or 4 phosphorylated residues in the phosphate centre with 0 or 1 phosphorylated residues in the minor cluster and these isomers are postulated to have a higher binding affinity for hydroxyapatite than the alternative isomers with only two phosphorylated residues in the phosphate centre and two in the minor cluster.

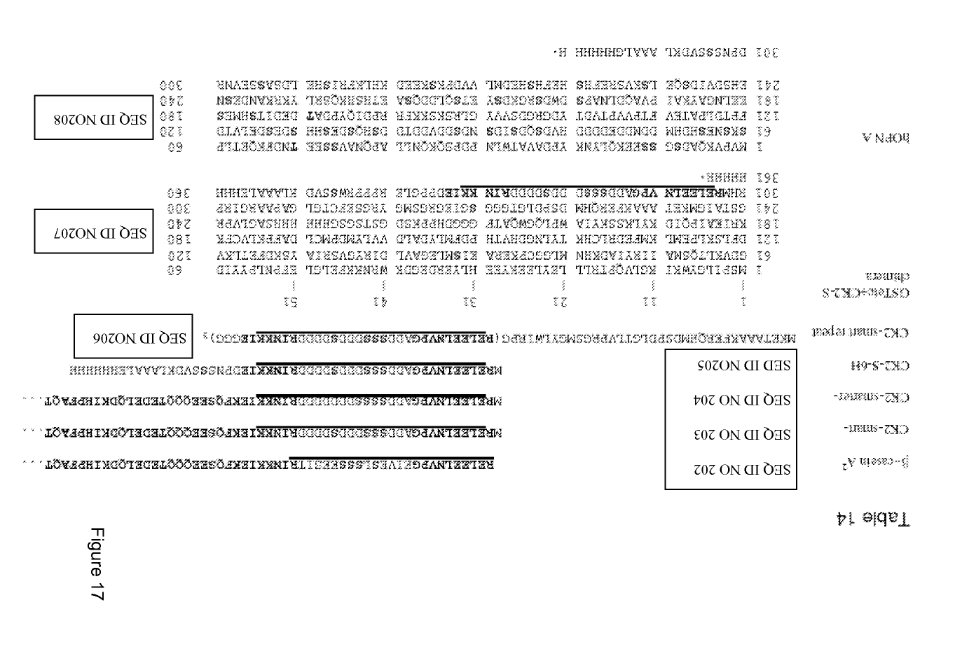

FIG. 15 is Table 6 which illustrates alignments of osteopontin phosphate centres 1-3.

FIG. 16 is Table 7 which illustrates the six potential phosphate centre-type sequences in DMP1 on the N-terminal side of the conserved cleavage site D-202.

FIG. 17 is Table 14 which illustrates sequences of recombinant multiply-phosphorylated proteins compared to native bovine β-casein A².

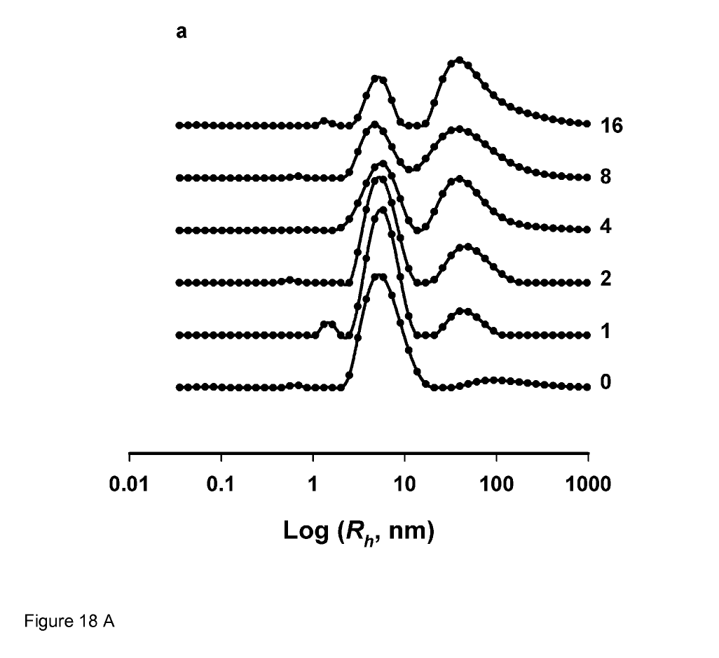

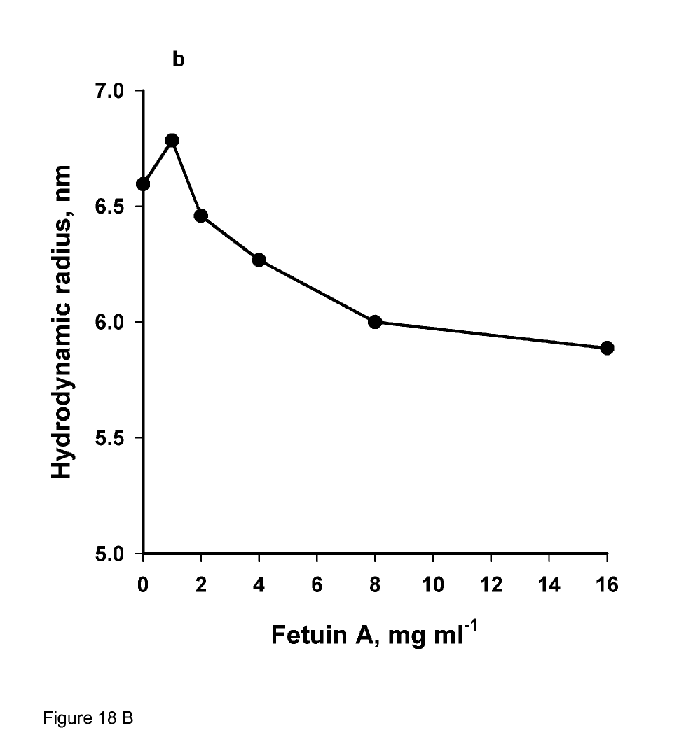

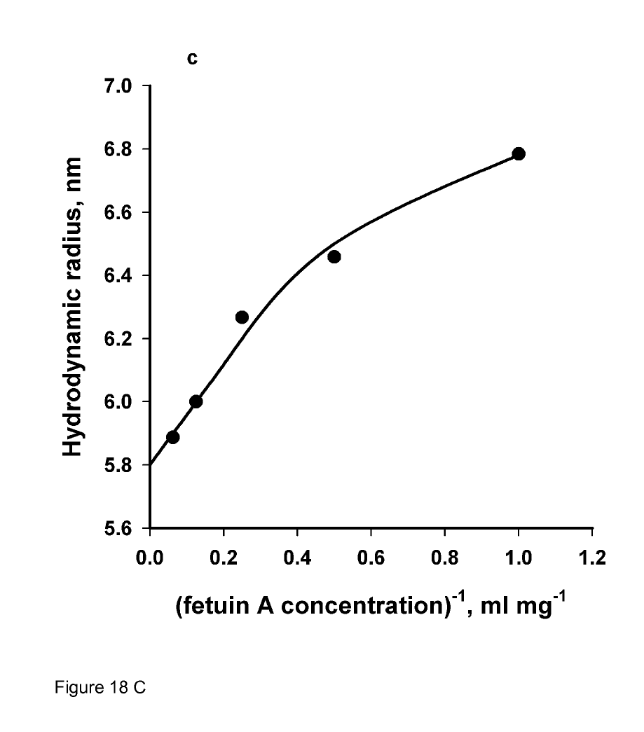

FIG. 18 a shows Intensity weighted hydrodynamic size distributions of nanoclusters formed from a casein phosphopeptide mixture with an increasing proportion of fetuin A, wherein the concentration of Fetuin A in mg ml⁻¹ is given against each distribution curve, and the distribution curves have been offset vertically for clarity.

FIG. 18 b shows the modal value of the hydrodynamic radius of the nanocluster peak versus the fetuin A concentration.

FIG. 18 c shows an extrapolation of the modal value of the nanocluster hydrodynamic radius to infinite fetuin A concentration wherein the intercept of the y axis gives the size of the pure fetuin A nanoclusters.

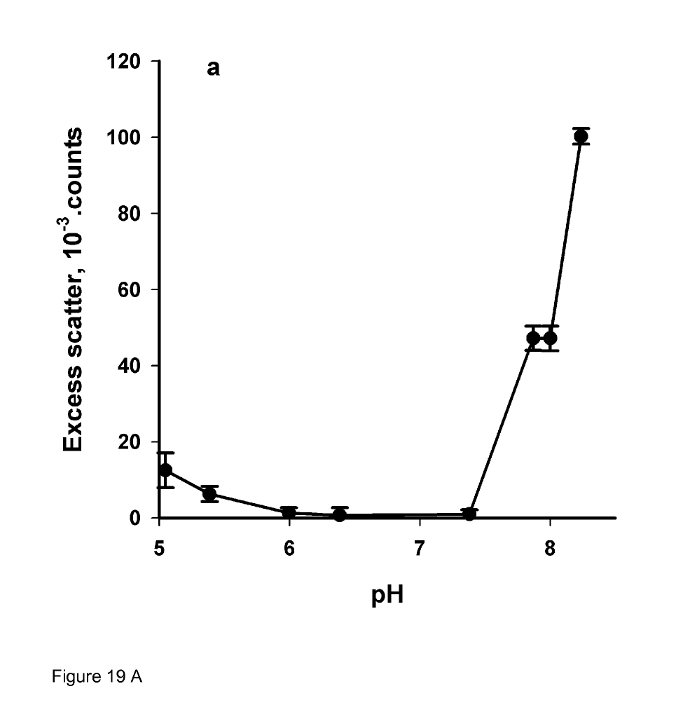

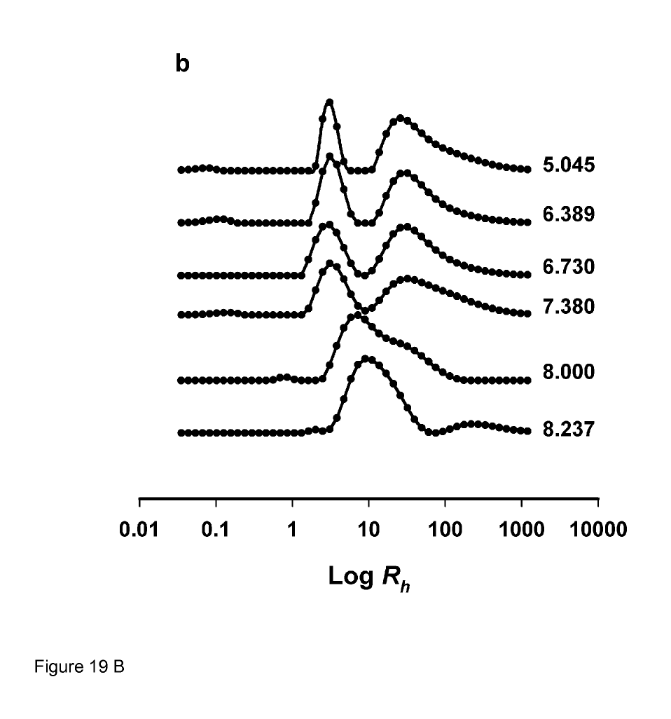

FIG. 19 shows dynamic light scattering by the stable artificial urine. (a) Total scattered intensity versus pH. (b) Typical intensity weighted size distributions in the approximate pH range 5-8. Individual distribution curves have been offset vertically for clarity.

### METHODS

### Thermodynamic Model of Calcium Phosphate Sequestration

The chemical formula of an electroneutral calcium phosphate nanocluster can be written as a multiple of its empirical formula containing a single phosphate centre (PC).

[Ca_{R}_{_{Ca}}H_{R}_{_{H}}(Pᵢ)_{R}_{_{P}}(H₂O)_{R}_{_{W}}(Pep-PC)₁]_{ī} (1)

The average molar ratios of water, calcium and inorganic phosphorus (Pᵢ) to the phosphate centre are R_{W}, R_{ca} and R_{P}, respectively, ī is the average number of phosphate centres in the nanocluster and Pep is the chemical formula of the peptide divided by the number of phosphate centres it contains. The formula of the monomer can be further divided into an amorphous hydrated calcium phosphate and a sequestering ligand of calcium phosphopeptide. The invariant empirical chemical formula of the electroneutral calcium phosphate is

$${Ca ( H P {O}_{4} )}_{y} {( P {O}_{4} )}_{\frac{2 - 2 y}{3}} \cdot x ( {H}_{2} O ) \qquad ( 2 )$$

where 3y/(2+y) is the mol fraction of Pᵢ in the di-anionic form. The empirical chemical formula of the average complex can then be written as

$$\begin{matrix}{[ {Ca ( H P {O}_{4} )}_{y} {( P {O}_{4} )}_{\frac{2 - 2 y}{3}} \cdot x ( {H}_{2} O ) ]}_{\frac{3 {R}_{P}}{2 + y}} \cdot [ {Ca}_{m} {{H}_{n} ( Pep - PC )}_{1} ] . & ( 3 ) \\ x = ( \frac{2 + y}{3} ) \frac{{R}_{W}}{{R}_{P}} & ( 4 )\end{matrix}$$

If the net charge on the peptide per phosphate centre is Z,

$$m = ( {R}_{Ca} - \frac{3}{2 + y} {R}_{P} ) \\ n = Z - 2 m \qquad ( 5 )$$

The best characterized CaP nanocluster is that formed by bovine β-casein 1-25 which has a mass, determined by sedimentation equilibrium, of 197 kDa, R_{Ca}=13.2, R_{P}=6.5 and y=0.4. This yields a monomer mass of 4246 Da and an average number of monomers of 46.3. The radius of the core was found by SANS to be 2.39 nm at the contrast match point of protein in the shell, which yields a core surface area per mole of PC (A_{core}) of 9.22.10⁹ cm² mol⁻¹. It can readily be shown by simple geometry that the average number of calcium phosphate molecules in a nanocluster, j̅, is given by

$$\overline{j} = ( \frac{3 {R}_{P}}{2 + y} ) \overline{i} = {( \frac{3 {kN}_{A} {R}_{P}}{( 2 + y ) {A}_{core}} )}^{3} \qquad ( 6 )$$

where k=(36πV_{core}²)^{1/3}, N_{A} is Avogadro's constant and V_{core} is the molar volume of the empirical formula of the calcium phosphate. From equation (6), j̅=374 and V_{core}=89.6 cm³ mol⁻¹, the core volume defined by SANS probably includes the peptide phosphate moieties and bound calcium, but is large enough also to contain about 10 water molecules per monomer (i.e. x in equation (3) is about 1.3). Previously, a value for j̅ of 350 was calculated assuming the core material had V̅_{core}=74.64 cm³ mole⁻¹, which is the molar volume of the nearest crystalline analogue of the amorphous and hydrated core, DCPD. This crystal structure had been used to model the short-range structure of the amorphous calcium phosphate in micellar calcium phosphate, as determined by X-ray absorption fine structure spectroscopy at the K-absorption edge of Ca.

### Free Energy of Formation of Calcium Phosphate Nanoclusters

The Gibbs free energy of formation of the nanoclusters (ΔG_{CPN}) can be divided into the free energy of formation of the core of CaP (ΔG_{core}) and the free energy of sequestration of the CaP in the surface by the phosphoprotein (ΔGₛₕₑₗₗ) to form the core-shell structure.

ΔG_{CPN}=ΔG_{core}+ΔGₛₕₑₗₗ (7)

The free energy of the CaP in the core, compared to a bulk phase (ΔG°_{core}) can be written in terms of the Kelvin equation as

$$Δ {G}_{core} - Δ {G}_{core}^{o} = RT ln ( \frac{{a}_{core}}{{a}_{s}} ) = \frac{2 {V}_{core} {γ}_{core}}{{r}_{core}} . \qquad ( 8 )$$

where γ_{core} and r_{core} are the interfacial tension and radius of the core and aₛ is the activity of the monomer in a saturated solution of the bulk phase. Clearly, if the core radius is a constant, the free energy of formation of the core is also constant. The empirical chemical formula of the calcium phosphate can then be used to define a solubility constant, K_{S}, in terms of the activities of the ions. In a dilute solution where the activity of water is effectively unity,

K_{S}=a_{Ca}_{²⁺}¹a_{HPO}_{₄}_{^{2−}}^{y}a_{PO}_{₄}_{^{3−}}⁽²⁻²y)/3 (9)

The formation constant can be used, just like the solubility product of a pure bulk phase, to calculate the extent of formation of the nanoclusters. Nevertheless, although the actual value of K_{S} is a constant for a given sequestering peptide, the form of the equation, and its numerical value depend on the sequestration free energy.

### Determination of Phosphopeptides Predicted to Form Nanoclusters

Based on the determination that the N-terminal peptide OPN 1-149, but not the whole protein, can form an equilibrium, nm-sized complex with calcium phosphate, the inventors have determined a general phosphate centre sequence motif and the conditions required for nanocluster formation. Using searches for secreted phosphoproteins, known to the inventors to be involved in calcium phosphate mineralization processes, in the UniProt (=Swiss-Prot+TrEMBL) database on the ExPASy server (www.expasy.com) of the Swiss Institute of Bioinformatics, possible nanocluster forming phosphopeptides were identified. Alignment of orthologous sequences was performed and the general motifs generated using the ClustalW2 method and the pattern searching routine PRATT, respectively, both implemented on the European Bioinformatics Institute server (www.ebi.ac.uk). In view of the remarkable sequence variability and the rarity of high scoring residues, standard scoring matrices applied to caseins were previously not found to work well without the use of supplementary constraints (Holt, C. & Sawyer, L. (1993). Caseins as Rheomorphic Proteins—Interpretation of Primary and Secondary Structures of the Alpha-S1-Caseins, Beta-Caseins and Kappa-Caseins. Journal of the Chemical Society-Faraday Transactions 89, 2683-2692). This was also found to be true of the other secretory calcium binding phosphoproteins and of all other secreted phosphoprotein sequences around phosphate centres. In view of this, alignments were made of sequences coded by individual exons or neighbouring exons, making use of the tendency for the conservation of splice junctions. Also, known and predicted sites of phosphorylation were edited to become Cys residues in order to give them a higher alignment score than Ser or Thr. Predicted sites of phosphorylation were identified according to the consensus rules for phosphorylation by the Golgi and casein kinase 2 kinases. Potential phosphate centre sequences were selected manually from the alignment of orthologues of eukaryotic proteins known to be involved in calcium phosphate mineralization. For comparative purposes, an exception was made of chicken riboflavin-binding protein which fulfils the sequence criteria, but is not at present known to be involved in mineralization. The identified phosphoproteins containing proven or candidate phosphate centres (mostly human sequences) are listed in Table 1. Further tables showing phosphate centres determined in a range of phosphopeptides are provided for illustrative purposes. As will be appreciated, phosphoproteins as identified in Table 1, which are present in other species may be utilised, where such sequences fit the parameters described above.

TABLE 1
Secreted phosphoproteins containing proven or candidate
phosphate centres

| | Swiss-Prot | |
|---|---|---|
| Gene name or | accession | |
| abbreviation | No.¹ | Protein name |
| SCPPs | | |
| OPN | P10451 | Osteopontin |
| CSN1S1 | P47710 | αₛ₁-casein |
| CSN1S2 | P02663 | αₛ₂-casein² |
| CSN2 | P05814 | β-casein |
| IBSP-II | P21815 | Integrin-binding sialophosphoprotein |
| MEPE | Q9NQ76 | Matrix extracellular bone |
| | | phosphoglycoprotein |
| DMP1 | Q13316 | Dentin matrix acidic phosphoprotein 1 |
| Non SCPPs | | |
| FETUA | P02765 | Fetuin A |
| PRB4 | P10163 | Proline-rich basic phosphoprotein 4 |
| MGP¹ | P08493 | Matrix Gla protein |
| SPP-24 | Q13103 | Secreted phosphoprotein 24 |
| RBP³ | P02752 | Riboflavin-binding protein |

Swiss-Prot entries are nearly all of human sequences and, where there are several isofoms to consider, isoform 1 has been chosen. ² SCCP is the abbreviation used for Secretory calcium binding phosphoprotein. ³Human CSN1S2 is a pseudogene so the bovine orthologue is given. ⁴Chicken RBP is the only RBP to contain a phosphate centre sequence. Example phosphate centre sequences are shown in Table 2.

TABLE 2
Candidate or confirmed phosphate centre sequences formed by
the action of the Golgi and CK2 kinases on selected secreted
phosphoproteins and the recombinant CK2-S and CK2-SS peptide.

| | | | | SEQ |
|---|---|---|---|---|
| | | Swiss- | | ID |
| Protein | Species | Prot No. | PC¹ | NO |
| OPN | Cow | P31096 | 1- LPVKPTSSGSSEEKQLNN-18 | 23 |
| | | | 42- QNSVSSEETDD -52 | 24 |
| | | | 96- PDHSDESHHSDESDEVD-112 | 25 |
| DMP1 | Mouse | O55188 | 4- ARYHNTESESSEERTDDL-21 | 26 |
| | | | 28- PTNSESSEESQA -39 | 27 |
| | | | 43- GQANSDHTDSSESGEEL -59 | 28 |
| | | | 116- SDEDSADTTQSSEDSTSQE-134 | 29 |
| | | | 139- DTPSDSKDQDSEDE -152 | 30 |
| | | | 161- DSAQDSESEE -170 | 31 |
| CSN1S1 | Guinea | P04656 | 16- RGGSSSSSSSEERLKEE-32 | 32 |
| | pig | | | |
| | | | 54- IISESTEEREA -64 | 33 |
| | | | 61- QREASSISSSEEVVPKN-77 | 34 |
| CSN1S2 | Pig | P39036 | 1- KHEMEHVSSSEESINIS-17 | 35 |
| | | | 54- ASSSSSEESVD -64 | 36 |
| | | | 125- IQSGEELSTSEEPVSSS-141 | 37 |
| CSN2 | Human | P05814 | 1- RETIESLSSSEESITEY-17 | 38 |
| IBSP | Man | P21815 | 50- SSEENGDDSSEEEEEE -63 | 39 |
| MEPE | Man | Q9NQ76 | 505- SSESSDSGSSSESDGD -525 | 40 |
| FETUA | Man | P02765 | 304- GVVSLGSPSGEVSHP-318 | 41 |
| SPP24 | Man | Q13103 | 108- SSSTSESYSSEEMIFG-123 | 42 |
| MGP | Man | P08493 | 1- YESHESMESYE -11 | 43 |
| PRB4 | Man | P10163 | 1- ESSSEDVSQEES -12 | 44 |
| RBP | Chicken | P02752 | 190-SESSEESSSMSSSEEHACQ-205 | 45 |
| CK2-SS | — | — | ELNVPGADDSSSSDDDDDD | 46 |
| CK2-S | — | — | ELNVPGADDSSSDDDSDDD | 47 |

¹Sites of phosphorylation by the Golgi or CK-2 kinases are shown as bold face.

TABLE 3
β-Casein phosphate centre alignment

| | | | SEQ |
|---|---|---|---|
| | Swiss- | β-PC | ID |
| Species | Prot No. | Exons 2-5¹ | NO |
| Cow | P02666 | RELEELNVPGEIVESL------SSSEESITRINK | 48 |
| Water | Q9TSI0 | RELEELNVPGEIVESL------SSSEESITHINK | 49 |
| buffalo | | | |
| Dromedary | Q9TVD0 | REKEEFKTAGEALESI------SSSEESITHINK | 50 |
| Sheep | P11839 | REQEELNVVGETVESL------SSSEESITHINK | 51 |
| Goat | P33048 | REQEELNVVGETVESL------SSSEESITHINK | 52 |
| Horse² | Q9GKK3 | REKEELNVSSETVESLSSNEPDSSSEESITHINK | 53 |
| Dog | Q9N2G8 | REKEELTLSNETVESL------SSSEESITHINK | 54 |
| Rabbit | P09116 | REKEQLSVPTEAVGSV------SSSEE-ITHINK | 55 |
| Pig | P39037 | RAKEELNASGETVESL------SSSEESITHISK | 56 |
| Mouse | P10598 | RETT-FTVSSET-DSI-------SSEESVEHINE | 57 |
| Rat | P02665 | REKDAFTVSSETG-SI-------SSEESVEHINE | 58 |
| Human | P05814 | RE---------TIESL------SSSEESITEYKQ | 59 |
| Tamar | P28550 | RP---------MVEKI------SESEEYVNEVPE | 60 |
| wallaby | | | |
| Bush-tailed | Q9XSE4 | RP---------MVEKI------SESEEHVTDVPE | 61 |
| possum | | | |

¹Exon numbering follows the bovine genes, ²Isoform 1

TABLE 4
α_{S1}-Casein phosphate centre alignments

| | Swiss-Prot | α_{S1}-PC-1 | ID |
|---|---|---|---|
| Species | No. | Exons 3, 3′ and 4¹ | NO |
| Cow | P02662 | KHPIKHQGLPQ--------------EVLNEN-LLRFFVA- | 62 |
| Water | O62823 | KQPIKHQGLPQ--------------GVLNEN-LLRFFVA- | 63 |
| buffalo | | | |
| Sheep | P04653 | KHPIKHQGLSP--------------EVLNEN-LLRFVVA- | 64 |
| Goat | P18626 | KHPINHQGLSP--------------EVLNEN-LLRFVVA- | 65 |
| Dromedary | O97943 | KYPLRYPEVFQNEPD------SIE-EVLNKRKILELAVVS | 66 |
| Pig | P39035 | KPPLRHQEHLQNEPD------SRE-ELFKERKFLRFPEV- | 67 |
| Rabbit | P09115 | KFHLGHLKLTQEQPE------SSEQEILKERKLLRF-VQ- | 68 |
| Guinea pig | P04656 | KFPFRHTELFQTQRGGSSSSSSSE-ERLKEENIFKF-DQ- | 69 |
| Rat | P02661 | RAHRRNAVSSQTQQEN----SSSE-EQEIVKQPKYLSLNE | 70 |
| Mouse | P19228 | RLHSRNAVSSQTQQQH----SSSE-EIFKQPKYLNLN-Q- | 71 |
| Human² | P47710 | KLPLRYPERLQNPSE------SSE---------------- | 72 |
| Tamar | P28549 | RELENRLNEDPIPVSEA---SSSE-ESVHQLNRDRRPLE- | 73 |
| wallaby | | | |
| | Swiss-Prot | α_{S1}-PC-2 | |
| Species | No. | Exons 6-8¹ | |
| Cow | P02662 | EKVNELSKDIGSESTEDQAMEDIK | 74 |
| Water | O62823 | EKVNELSTDIGSESTEDQAMEDIK | 75 |
| buffalo | | | |
| Sheep | P04653 | ENINELSKDIGSESIEDQAMEDAK | 76 |
| Goat | P18626 | ENINELSKDIGSESTEDQAMEDAK | 77 |
| Dromedary | O97943 | ENIDEL-KDTRNEPTEDHIMEDTE | 78 |
| Pig | P39035 | EIINELNR---------------- | 79 |
| Rabbit | P09115 | EKENEEIKGTRNEVTEEHVLADRE | 80 |
| Guinea pig | P04656 | -KQSEKIKEIISESTEE------- | 81 |
| Rat | P02661 | EQDN-EIKITMDSSAEEQATASAQ | 82 |
| Mouse | P19228 | EQND-EIKVTMDAASEEQAMASAQ | 83 |
| Human² | P47710 | EKQTDEIKDTRNESTQNCVVAEPE | 84 |
| Tamar | P28549 | YELDKYREDLKTSSSEEFVTPSTN | 85 |
| wallaby | | | |
| | Swiss-Prot | α_{S1}-PC-3 | |
| Species | No. | Exons 9 and 10¹ | |
| Cow | P02662 | QMEAESISSSEEIVPNSVE--- | 86 |
| Water | O62823 | QMEAESISSSEEIVPISVE--- | 87 |
| buffalo | | | |
| Sheep | P04653 | QMKAGSSSSSEEIVPNSAE--- | 88 |
| Goat | P18626 | QMKAGSSSSSEEIVPNSAE--- | 89 |
| Dromedary | O97943 | RKE-SGSSSSEEVVSSTTE--- | 90 |
| Pig | P39035 | Q-RGSSSSSSEEVVGNSAE--- | 91 |
| Rabbit | P09115 | -TEASISSSSEEIVPSSTK--- | 92 |
| Guinea pig | P04656 | QREASSISSSEEVVPKNTE--- | 93 |
| Rat | P02661 | EDSSSSSSSSEESKDAIPSATE | 94 |
| Mouse | P19228 | EDS-SISSSSEESEEAIPNITE | 95 |
| Human² | P47710 | KMESSISSSSEEMSLSKCA--- | 96 |
| Tamar | P28549 | NFNEEDSSASRERKIEDFSE-- | 97 |
| wallaby | | | |

¹Exon numbering follows the bovine genes, ²Isoform 1.

TABLE 5
Alignment of ase-casein phosphate centres

| | Swiss- | | SEQ |
|---|---|---|---|
| | Prot | α_{S2}-PC-1 | ID |
| Species | No. | Exons 2(part)-4 | NO |
| Cow | P02663 | KNTMEHVSSSEESI-ISQE | 98 |
| Sheep | P04654 | KHKMEHVSSSEEPINISQE | 99 |
| Goat | P33049 | KHKMEHVSSSEEPINIFQE | 100 |
| Dromedary | O97944 | KHEMDQGSSSEESINVSQQ | 101 |
| Pig | P39036 | KHEMEHVSSSEESINISQE | 102 |
| Rabbit B | P50419 | KPKIE-QSSSEETIAVSQE | 103 |
| Rabbit A | P50418 | KNGIEQRSASEEIVSFYQE | 104 |
| Mouse A | Q02862 | KHEIKDKSSSEESSASIYP | 105 |
| Mouse B | P02664 | KQRMEQYISSEESMDNSQE | 106 |
| Guinea pig | P04655 | KHKSEQQSSSEESVSISQE | 107 |
| Rat A | P02667 | KHAVKDKPSSEESASVYLG | 108 |
| | Swiss- | | |
| | Prot | α_{S2}-PC-2 | |
| Species | No. | Exons 7-9 | |
| Cow | P02663 | EVVRNANEE-EYSIGSSSEESAEVATE | 109 |
| Sheep | P04654 | EVVRNADEE-EYSIRSSSEESAEVAPE | 110 |
| Goat | P33049 | EVVRNANEE-EYSIRSSSEESAEVAPE | 111 |
| Dromedary | O97944 | EAVRNIKEV----------ESAEVPTE | 112 |
| Pig | P39036 | EAVRNIKEV-GYASSSSSEESVDIPAE | 113 |
| Rabbit B | P50419 | EPIKNINEV----------EYVEVPTE | 114 |
| Rabbit A | P50418 | ---------QETPSVSSSEESVEVQTE | 115 |
| Mouse A | Q02862 | ----------DSASSSSSEESSEEVSE | 116 |
| Mouse B | P02664 | ETVENIYIP------------------ | 117 |
| Guinea pig | P04655 | EATKNTPKM-AFFSRSSSEEFADIHRE | 118 |
| Rat A | P02667 | ----------DSASSSSSEESSEEISE | 119 |
| | Swiss- | | |
| | Prot | α_{S2}-PC-3 | |
| Species | No. | Exons 12-14 | |
| Cow | P02663 | NREQLSTSE--------ENSKKTVDM | 120 |
| Sheep | P04654 | NREQLSTSE--------ENSKKTIDM | 121 |
| Goat | P33049 | NREQLSTSE--------ENSKKTIDM | 122 |
| Dromedary | O97944 | NTEQLSISEESTEVPTE--------- | 123 |
| Pig | P39036 | SGEELSTSEEPVSSSQEENT-KTVDM | 124 |
| Rabbit B | P50419 | -------------------------- | |
| Rabbit A | P50418 | -------------------------- | |
| Mouse A | Q02862 | ---------ESISTSVEEILKKIIDM | 125 |
| Mouse B | P02664 | -------------------------- | |
| Guinea pig | P04655 | ---------ESSSSSSTEKSTDVFIK | 126 |
| Rat A | P02667 | ---------ESTSTSVEEILKKIIDI | 127 |

Additional Tables 6 and 7 are provided in the figures.

TABLE 8
Alignment of phosphate centres in IBSP-II coded
by exon 5 showing confirmed in vivo
phosphorylation of the bovine sequence

| | | | SEQ |
|---|---|---|---|
| | Swiss- | | ID |
| Species | Prot No. | | NO |
| Man | P21815 | GSSDSSEENGD-DSSEEEEEEEETS | 128 |
| Rat | P13839 | GGSDSSEENGDGDSSEEEGEEEETS | 129 |
| Mouse | Q61711 | GGSDSSEENGDGDSSEEEGEEEETS | 130 |
| Cow | Q28862 | SSSDSSEENGNGDSSEEEEEEEETS | 131 |
| Pig | P31936 | SSSDSSEENGNGDSSEEEEEEEENS | 132 |
| Chicken | P79780 | G-SDSSEEEG--DGSEEEEEGGAPS | 133 |

TABLE 9
Alignment of C-terminal phosphate centre of MEPE
showing predicted sites of phosphorylation

| | | | SEQ |
|---|---|---|---|
| | Swiss-Prot | | ID |
| Species | No. | | NO |
| Macaque | Q9GM13 | RREDSSESSDSGSSSESDGD-555 | 166 |
| Man | Q9NQ76 | RRDDSSESSDSGSSSESDGD-525 | 167 |
| Mouse | Q3TYZ5 | QR-DSSESSSSGSSSESHGD-433 | 168 |
| Rat | Q8K3V0 | QR-DSSESSSSGSSSESSGD-435 | 169 |

TABLE 10
Alignment of phosphate centre sequences in
Fetuin A

| | Swiss- | | SEQ |
|---|---|---|---|
| | Prot | | ID |
| Species | No | Sequence | NO |
| Cow | P12763 | FSGVASVESSSGE 307 | 170 |
| Sheep | P29701 | FSGVASVESASGE 315 | 171 |
| Pig | P29700 | FSGVASVESASGE 310 | 172 |
| Man | P02765 | FMGVVSLGSPSGE 304 | 173 |
| Mouse | P29699 | FSPVASVESASGE 298 | 174 |
| Rat | P24090 | FSPVASVESASGE 302 | 175 |
| Chimpanzee | Q9N2D0 | FMGVVSLGSPSGE 304 | 176 |
| Guinea pig | 070159 | FP---DVDSASGE 304 | 177 |
| Rabbit | P80191 | FAGVPSMESGSGE 307 | 178 |
| Gerbil | P97515 | FSPVASVESASGE 300 | 179 |

TABLE 11
Alignment of phosphate centre sequences in SPP-24

| | | | SEQ |
|---|---|---|---|
| | Swiss- | | ID |
| Species | Prot No | Sequence | NO |
| Cow | Q27967 | CHWSS-SSGSSSSEEM 129 | 180 |
| Sheep | Q70TH4 | CHWSS-SSGSSSSEEM 129 | 181 |
| Pig | Q711S8 | CRWSS-SSESNSSEEM 129 | 182 |
| Man | Q13103 | CSWSSSTSESYSSEEM 126 | 183 |
| Mouse | Q8K1I3 | CRWAS-SSESNSSEEM 128 | 184 |
| Rat | Q62740 | CRWAS-TSESNSSEEM 128 | 185 |
| Chicken | Q710A0 | CHQSTFSSESMSSEEM 126 | 186 |
| Trout | Q70I47 | CGQDSSSSES-SSEEN 119 | 187 |
| Salmon | Q710A1 | CGQDSSSSES-SSEEN 120 | 188 |

TABLE 12
Alignment of phosphate centre sequences in MGP¹
and phosphate centres in human PRB4 and chicken
RBP

| | Swiss- | | SEQ |
|---|---|---|---|
| | Prot | | ID |
| Species | No | Sequence | NO |
| Cow | P07507 | YESHESLESYE 11 | 189 |
| Pig | Q8MJ39 | YESHESLESYE 11 | 190 |
| Man | P08493 | YESHESMESYE 11 | 191 |
| Mouse | P19788 | YESHESMESYE 11 | 192 |
| Rat | P08494 | YESHESMESYE 11 | 193 |
| Rabbit | P47841 | YESHESMESYE 11 | 194 |
| Chicken | O42413 | YESHESMESHE 11 | 195 |
| Orangutan | Q5RDP6 | YESHESMESYE 11 | 196 |
| Chinchilla | Q6QN06 | YESHESMESYE 11 | 197 |
| Meagre | Q800Y2 | YESHESSESAE 11 | 198 |
| Shark | P56620 | -DSSESNEIED 10 | 199 |
| PRB4 | P10163 | ESSSEDVSQEESLF | 200 |
| Man | | LISGK-19 | |
| RBP | P02752 | SESSEESSSMSSSE | 201 |
| Chicken | | EHACQ-205 | |

¹In MGP, E-2 is normally γ-carboxylated to Gla.

TABLE 13
Recombinant nanocluster-forming peptides

| | |
|---|---|
| CK2-SS | --ELEELNVPGADDSSSSDDDDDDD RINKK |
| CK2-S | --ELEELNVPGADD-SSSDDDSDDDDRINKK |
| CK2-S-6H | MRELEELNVPGADD-SSSDDDSDDDDRINKKIEDPNSSSVDKLAAALEHHHHHH |

### Prediction of Flexible Sequences

To determine whether predicted phosphate centres are provided in flexible sequence portions of the phosphopeptides identified, predictions were made using the PONDR® VL-XT predictor (http://www.pondr.com/; which integrates three feed-forward neural networks: the VL1 predictor, the N-terminus predictor (XN), and the C-terminus predictor (XC). VL-XT outputs are real numbers between 1 and 0, where 1 is the ideal prediction of disorder and 0 is the ideal prediction of order. VL-XT outputs are typically not exact and a threshold is applied with disorder assigned to values greater than or equal to 0.5. Predictions of long flexible regions of 40 or more residues are considered more reliable than shorter regions.

### Size Distribution of Nanoclusters

Since iₘₐₓ must be a positive real number, two possible solutions exist. In classical nucleation theory the surface energy is positive and the bulk free energy term is negative; precipitation occurs from a supersaturated solution in which a₁>aₛ. In the formation of the nanoclusters, the effective surface energy is negative and hence the solution is undersaturated with respect to the bulk phase (a₁<aₛ). Note that the bulk phase in this context is amorphous calcium phosphate. When a fluid volume of V litres containing a phosphopeptide or phosphoprotein having f competent phosphate centres in its sequence at a molar concentration [PP] is considered, if p moles of calcium phosphate (CaP) precipitate from the solution in a localised fluctuation from equilibrium, then the condition for thermodynamic stability in the whole volume of fluid is

$$α = \frac{p}{f [ PP ] {R}_{P} V} < 1 \qquad ( 10 )$$

where α is the fraction of reacted phosphate centres and V is the total volume of fluid. Clearly there must be an excess of the phosphate centres for the fluid to be stable (α<1). Thermodynamic stability can be achieved through the formation of nanoclusters, while maintaining a state of supersaturation with respect to hydroxyapatite. Equation (18) places no upper limit on the concentrations of Ca and Pᵢ in the biofluid, which has been exploited in the formation of some milks with concentrations of total Ca in excess of 100 mM. Nevertheless, the free Ca ion concentrations and supersaturation with respect to hydroxyapatite remain comparable to those in blood.

It is considered that in a two phase system of amorphous calcium phosphate and a solution of a competent sequestering peptide at a high enough concentration, nanoclusters will form spontaneously at the expense of the amorphous calcium phosphate. The inventors believe this could be exploited in the removal of freshly formed ectopic deposits in soft tissues or in preventing the growth of amorphous calcium phosphate nuclei into colloidal or macroscopic crystalline particles in biofluids. Alternatively, as in a two phase system of hydroxyapatite and a nanocluster solution, where the hydroxyapatite will tend to grow at the expense of the nanoclusters; a nanocluster solution could act as a reservoir of Ca and Pᵢ for the growth or remineralization of hard tissues. The inventors do not consider that adding a protein or peptide containing an amino-acid sequence that promotes the maturation of amorphous calcium phosphate (ACP) to hydroxyapatite (HA) would destabilise the solution since the only route for the formation of hydroxyapatite is via the formation of amorphous calcium phosphate.

### Example 1

### Fractionation of the OPNmix Sample by Gel Filtration Chromatography and Composition

An osteopontin fraction (OPNmix) was isolated from bovine milk by the method of Sørensen et al. (Sørensen, E. S., Hojrup, P. & Petersen, T. E. (1995). Posttranslational Modifications of Bovine Osteopontin—Identification of 28 Phosphorylation and 3 O-Glycosylation Sites. Protein Science 4, 2040-2049) and Sorensen, E. S., & Petersen, T. E. (1993) Purification and characterisation of three proteins isolated from the proteose peptone fraction of Bovine Milk. J. Dairy Res 60: 189-197). Three major fractions from the proteose peptone fraction of bovine milk were isolated by Sephadex G-75 gel chromatography followed by Q-Sepharose ion-exchange chromatography in the presence of urea. From their mobility in a gradient SDS-PAGE. The proteins were found to have apparent molecular masses of 17, 28 and 60 kDa. The 60 kDa protein was collected and freeze dried. It comprised more than 95% osteopontin or osteopontin peptides, phosphorylated to some degree at all sites. It was fractionated further by Superdex 75 gel filtration chromatography using a Pharmacia XK 16 column having a bed length of 64 cm at a flow rate of 0.3 ml min⁻¹. The sample of 10 mg was dissolved in 1 ml of the elution buffer (50 mM Phosphate, 300 mM NaCl, 0.02% NaN₃, pH 7.0) and dialysed overnight against 120 ml of elution buffer before loading on the column. Fractions of 1 ml were collected and examined by SDS-MOPS gel electrophoresis (Qi, X. L., Holt, C., McNulty, D., Clarke, D. T., Brownlow, S. & Jones, G. R. (1997). Effect of temperature on the secondary structure of beta-lactoglobulin at pH 6.7, as determined by CD and IR spectroscopy: A test of the molten globule hypothesis. Biochemical Journal 324, 341-346 and McClenaghan, M., Hitchin, E., Stevenson, E. M., Clark, A. J., Holt, C. & Leaver, J. (1999). Insertion of a casein kinase recognition sequence induces phosphorylation of ovine beta-lactoglobulin in transgenic mice. Protein Engineering 12, 259-264). These were then pooled into 4 fractions (F1, F2, F3a and F3b) and dialysed exhaustively against deionised water before being freeze dried and the recovered weights recorded. The procedure was repeated as required to collect enough of each fraction for the physico-chemical studies.

As illustrated in FIG. 1a, the gel filtration chromatogram showed a main peak (F3) with some obvious substructure and two very much smaller peaks on the leading edge, labelled F1 and F2. Fractions of 1.0 ml were collected and examined by SDS gel electrophoresis (illustrated in FIG. 1b). None of the F1 sample penetrated the gel suggesting it was void volume material of M>200,000 Da. The F2 fraction was a single band with an apparent molecular weight of 50-55,000 Da and the F3 samples contained two main groups of bands which were pooled as F3a and F3b. The wt % of F2, F3a and F3b from the recovered weights were 10.2, 56.7 and 33.1, respectively. MALDI-MS measurements and calculations showed that F2 was the full length form (OPN 1-262) and the F3a fraction was the N-terminal fragment OPN 1-149, probably formed by the action of the principal milk proteinase plasmin. Full length native OPN had an experimentally determined molecular weight of 33.9 kDa, of which 1.7 kDa was due to phosphate groups and approx. 2.9 kDa was due to O-bound glycans. For the N-terminal fragment in F3a, the corresponding figures were 19.8 kDa with contributions of 0.9 kDa from phosphate groups and 2.9 kDa from the glycans. Thus, the average degree of phosphorylation of the 28 potential phosphorylation sites in the whole protein was 79%. The N-terminal fragment analysis was consistent with an average of about 60-65% phosphorylation of the 16 sites in an N-terminal plasmin peptide. All the glycans (3-4 sites) were present in both.

### Example 2

### Preparation of Calcium Phosphate Nanoclusters

### Urea/Urease Method

Nanoclusters were prepared by the urea/urease method, as discussed above and as known in the art (Holt, C., Wahlgren, N. M. & Drakenberg, T. (1996). Ability of a beta-casein phosphopeptide to modulate the precipitation of calcium phosphate by forming amorphous dicalcium phosphate nanoclusters. Biochemical Journal 314, 1035-1039) but wherein the phosphopeptide was provided by either the OPNmix, discussed above, or fractions from the gel filtration separation. Typically, an OPNmix sample (25 mg) was dissolved in 1 ml of water and dialysed overnight against 500 ml of 1 mM EDTA followed by exhaustive dialysis against deionised water to remove Ca ions. The Ca-free peptides were recovered by freeze drying.

An initial under-saturated solution of salts at pH 5 with the composition 22 mM Ca(NO₃)₂, 20 mM KH₂PO₄, 36 mM KNO₃ and 1.5 mM NaN₃ (Mg-free Buffer A) was used to dissolve the peptide. Sufficient urea (0-30 mM) was added from a stock solution of 200 mM to give the desired final pH after hydrolysis with 2 units of urease from Canavalia ensiformis (Calbiochem product no. 666133). These conditions allowed the pH to rise to within 0.004 units of the target value within a few minutes. The standard concentration of OPNmix and F3a was either 25 or 30 mg ml⁻¹; For the SAXS, electrophoretic light scattering and differential scanning calorimetry measurements, the OPN 1-149 nanoclusters were diluted to a suitable concentration of 5-10 mg ml⁻¹ with a buffer that preserved their integrity. The dilution buffer had the same salt composition and pH as an ultrafiltrate prepared from the nanocluster solution but with the addition of sodium azide (1.5 mM) as a preservative and 0.01% of the whole casein tryptic phosphopeptide mixture to inhibit calcium phosphate precipitation in the buffer during storage.

### Simple Mixing Method

Experiments were performed using the OPNmix sample at a final concentration of 30 mg ml⁻¹. At this concentration there was no initial precipitation even with a single addition of the P, stock provided it was added slowly with good stirring. The initial turbidity slowly disappeared over about a week to give a slightly opalescent solution comparable to that of nanoclusters prepared by the urea/urease method. When the peptide concentration was reduced to below 10 mg ml⁻¹, an initial colloidal precipitate did develop which did not fully re-disperse on standing. If, however, further OPNmix was added to a final concentration of 30 mg ml⁻¹ soon after the development of the initial turbidity, the solution clarified completely over about a week. However, if the addition of the phosphopeptide was delayed, or if the initial peptide concentration was below 5 mg ml⁻¹, complete re-dispersion was not achieved, even after 4 months.

The spontaneous re-dispersion of the amorphous calcium phosphate produced by the mixing method and the facile preparation of the nanoclusters by the urea/urease method demonstrates that the nanoclusters can be formed by both a forward reaction from a supersaturated solution and by a back reaction with a pre-formed precipitate of amorphous calcium phosphate. However, if the amorphous calcium phosphate was allowed to mature, even for only a few minutes, the osteopontin peptide was unable to disperse it. The standardised conditions for forming the equilibrium calcium phosphate nanoclusters by the urea/urease method yielded solutions that were stable for years. An OPN 1-149 nanocluster solution can therefore be regarded as thermodynamically stable with respect to phase separation of amorphous calcium phosphate, particularly if there is an excess of the free peptide.

### Example 3

### Partition of Salts by Ultrafiltration

Nanocluster solutions of the Ca-free OPNmix in the Mg-free Buffer A were prepared by the urea/urease method with a pH between 5.0 and 7.5. They were allowed to equilibrate before ultrafiltration through Vivaspin 0.5 ml concentrators (Product VS0101 with a molecular weight cut off of 10,000 Da, Vivascience AG, Germany) using a centripetal field of 5000×g for 15 min. The concentrations of Ca, free Ca²⁺, and P, in the ultrafiltrate and starting solution were determined (Little, E. M. & Holt, C. (2004). An equilibrium thermodynamic model of the sequestration of calcium phosphate by casein phosphopeptides. European Biophysics Journal with Biophysics Letters 33, 435-447). The concentrations of complexed Pᵢ and Ca, [Pᵢ]_{c} and [Ca]_{c}, respectively, were calculated from the difference between the total and ultrafiltrate concentrations after allowing for a Donnan equilibrium of each diffusible ion species across the semi-permeable membrane (Holt, C. (1997). The milk salts and their interaction with casein. In Advanced Dairy Chemistry Second edit. (Fox, P. F., ed.), Vol. 3, pp. 233-256. Chapman & Hall, London). The peptide-free ultrafiltrate composition was then used to calculate the ion equilibria (Holt, C., Dalgleish, D. G. & Jenness, R. (1981). Inorganic Constituents of Milk 0.2. Calculation of the Ion Equilibria in Milk Diffusate and Comparison with Experiment. Analytical Biochemistry 113, 154-163) and ion activity product for CaP, according to equation (9) for y in the range 0 (tri-calcium phosphate) to 1 (di-calcium phosphate). The ion activity product in the ultrafiltrates were calculated at each pH for a range of y values in order to find the value that was independent of pH. In FIG. 3a the results are shown after being divided by the average for each value of y, and a constant added to separate the different curves. The slope of each curve was found by linear regression and an invariant ion activity product, corresponding to zero slope FIG. 3b) was found for a tri-calcium phosphate stoichiometry (y=0, K_{S}=7.6.10⁻¹⁰ M^{1.66′}). This is a more basic ACP than that found in the casein nanoclusters which have y=0.4.

### Example 4

### Binding of Calcium Ions to OPN 1-149

To model the chemical species in the OPN 1-149 nanocluster solution requires the calculation of Ca ion binding to the free peptide at any pH in the range 5.0-8.0. A semi-empirical model was used to describe the binding isotherms obtained previously for the β-casein 1-25 phosphopeptide in this pH range and the same model was adapted to fit the binding isotherm of OPN 1-149 measured at pH 7.0. The rescaled model was then used to predict binding at any other value of the pH. Binding of Ca ions to the β-casein 1-25 peptide is predominantly to 4 phosphorylated residues and, to a lesser extent, to 7 Glu residues and the C-terminus.

Fitting of the experimental isotherms was done to an equation of the form

$${\overline{υ}}_{Ca} = {\sum }_{i} \frac{{ϕ}_{i} {N}_{i} ( pH ) {K}_{a , i} [ {Ca}^{2 +} ]}{( 1 + {K}_{a , i} [ {Ca}^{2 +} ] )} \qquad ( 11 )$$

where the summation is over all binding sites with Ca ion association constants K_{a,i}. The function Nᵢ(pH) describes the number of sites of type i as a function of pH and φᵢ is the degree of phosphorylation. It was assumed, that all the phosphorylated sites had the average degree of phosphorylation φ̅. The number of dianionic phosphoseryl sites at a given pH is given by

N_{P}_{^{2−}}= φ̅N_{P}/(1+10^{pK-PH}) (12)

The titration behaviour of casein phosphate groups in a phosphate centre of the type -sPXsPsPsPEE- shows two effective pKs associated with the consecutive cluster of three sites (pK=6.7) whereas the other site (pK=5.95) is more typical of an isolated residue. Since the sites are not independent, the effective pKs will be influenced by the binding of Ca ions.

The binding behaviour of β-casein has shown that one Ca ion is bound much more strongly than the rest, so the model for binding to the phosphate dianionic groups allowed for two pKs and two Ca ion association constants. The phosphorylated sites have a reduced affinity for Ca ions when they become protonated and were treated as having the same low affinity as the carboxyl groups. The pK of the first ionisation of the phosphate groups does not influence binding in the pH range of interest and hence was omitted from the model. The OPN peptide has 16 sites of phosphorylation, 60% of which were actually phosphorylated, and 37 carboxyl groups, including the C-terminus. However the sequence does not contain the three consecutive phosphorylated residues of the β-casein phosphate centre. It proved possible to reduce the number of types of site to two, namely high affinity dianionic phosphoseryl sites titrating at a pK of 6.4 and the low affinity sites comprising the carboxyl groups and protonated phosphoseryl residues.

The Ca²⁺ binding isotherm of peptides recovered from fraction F3a was determined by measuring the concentration of free Ca ions with a Ca ion selective electrode after successive small additions of a stock solution of 100 mM Ca(NO₃)₂ to a solution containing 25 mg ml⁻¹ of F3a, buffered to pH 7.0 with 20 mM P, and 50 mM KNO₃. The total Ca concentration ranged from 0 to 19.3 mM and the corresponding ionic strengths varied from 80 to 93 mM. The three pKs and Ca ion association constants were allowed to vary during the simultaneous fitting to the experimental isotherms of the β-casein 1-25 peptide and the resulting fitted curves are shown in FIG. 2. One high affinity site was generated by the ionisation of the phosphate moieties in the consecutive cluster of three sites along with two sites of lower affinity and a third lower affinity site resulted from the ionisation of the isolated phosphoseryl residue. The lowest affinity of sites resulted from the combination of carboxyl and protonated phosphoseryl residues. The calculated OPN 1-149 isotherm was obtained by a separate fitting of the single isotherm employing a model with two pKs and association constants but keeping as many as possible of the constants from the casein model.

### Example 5

### Calculation of the Chemical Species in Nanocluster Solutions

At a given fraction of reacted phosphate centres, the concentrations of non-diffusible (complexed) Ca and P, are given by

[Pᵢ]_{c}=f[PP]R_{P}α

[Ca]_{c}=f[PP](R_{Ca}α+(1−α) ν̅_{Ca}([Ca²⁺],pH)) (13)

where [PP] is the phosphopeptide concentration. From these values the diffusible concentrations were calculated and hence the composition of the ultrafiltrate was obtained from the assumed Donnan equilibrium across the membrane. Below pH 5.97, no nanoclusters could form because the ion activity product was below K_{S}; the equilibria involve only small ions, ion complexes and ion binding to the peptides. Above pH 5.97, the extent of reaction of the phosphate centres was found which allowed the ion activity product in the nanocluster solution to equal K_{S}, assuming the same values for R_{Ca} and R_{P} as were found for casein phosphopeptide nanoclusters. Peptide binding was modelled as if all the peptides had the same properties as OPN 1-149, even though the weight fraction of this latter peptide is less than 60% of the total. The complete model of the ion equilibria was then used to calculate the composition of an equilibrium diffusate, so that it could be compared with the composition of the experimental ultrafiltrate (FIG. 4a). FIG. 4 b shows how the calculated extent of reaction of the PCs varied with pH. The general agreement of the model with experiment is satisfactory although the calculated free Ca ion concentration is systematically below experiment at the lower pH values. At a given extent of reaction, the fraction of free peptide depends on whether the 3 phosphate centres react independently or together in sequestering the calcium phosphate. If they are not independent then the fraction of free peptide is (1−α) but if they react completely independently then the fraction of peptide chains with no reacted PCs is (1−α)³. In addition to the work with the OPNmix sample, a single determination was made of the partition of salts in a nanocluster solution at pH 7.0 prepared with the pure OPN 1-149 peptide. The experimental and, in parentheses, the model, ultrafiltrate concentrations of Pᵢ, Ca and Ca²⁺ were 12.1 (11.1), 1.4 (0.92) and 1.1 (0.52) mM, respectively, which compare quite closely with values obtained with the OPNmix.

### Example 6

### Structural Models

In a SAXS experiment, wherein measurements were made on station 2.1 at the CCLRC Daresbury Laboratory, the normalised scattered intensity I(q) was measured. For a dispersion of isotropic, monodisperse particles, I(q) is related to the inter-particle structure factor, S(q) and the particle scattering factor P(q) by

$$I ( q ) = Δ {\overline{ρ}}_{p}^{2} \frac{NM}{{V}_{p}} P ( q ) S ( q ) \qquad ( 14 )$$

where N is the number of particles of mean excess scattering length density over the solvent Δ ρ̅ₚ, volume Vₚ and mass M. The scattering wave vector is q=4π sin θ/λₒ, where θ is half the scattering angle and λₒ is the wavelength of the X-rays. In a dilute solution where S(q)=1 and a fraction α′ of the peptide chains have formed a polydisperse distribution of nanoclusters, equation (22) becomes

$$I ( q ) = \frac{{α}^{′} N}{f \overline{i}} {\sum }_{i} Δ {\overline{ρ}}_{CPN , i}^{2} \frac{{x}_{i} {M}_{CPN , i}}{{V}_{CPN , i}} {P}_{CPN , i} ( q ) + {Δρ}_{chain}^{2} \frac{( 1 - {α}^{′} ) {NM}_{chain}}{{V}_{chain}} {P}_{chain} ( q ) \qquad ( 15 )$$

where N is the number of peptide chains.

### Scattering Length Densities

The molar volumes of the unphosphorylated residues were taken from Jacrot and Zaccai (Jacrot, B. & Zaccai, G. (1981). Determination of Molecular-Weight by Neutron-Scattering. Biopolymers 20, 2413-2426). The molar volume of ortho-L-phosphoserine was calculated from the unit cell volume of the amino acid, as determined by Sundaralingam (Sundaralingam, M. & Putkey, E. F. (1970). Molecular structures of amino acids and peptides. II. A redetermination of the crystal structure of L-O-serine phosphate. A very short phosphate-carboxyl hydrogen bond. Acta Crystallographica B26, 790-800) and 11 Å³ subtracted to give the residue volume of 168.7 Å³. The difference in residue volumes of serine and phosphoserine was then used to calculate a residue volume for phosphothreonine of 191.7 Å³. The X-ray forward scattering amplitudes of the residues were calculated from the elemental compositions using atomic scattering factors. For OPN 16P 1-149 and OPN 28P 1-262, the scattering length densities in units of 10⁻¹⁰ cm⁻² were calculated to be 12.56 and 12.48, respectively. For the nanoclusters prepared with OPN 16P 1-149 it was assumed that the phosphate moieties of the 10 phosphorylated residues found in the three PCs, were assigned to the core, giving ρₛₕₑₗₗ=12.40×10¹⁰ cm⁻². For the core, a hydrated CaP with the composition given by equation (2) gave ρ_{core}=19.1×10¹⁰ cm⁻². Radii of gyration of the whole protein and N-terminal polypeptide were independent of concentration in the range 5-15 mg ml⁻¹. For OPN the average of three determinations by the Guinier method was 5.50±0.17 nm and the corresponding value for OPN 1-149 was 2.17±0.24 nm. For OPN with 262 residues, equation (28) predicts a radius of gyration of 5.38 nm and for the N-terminal plasmin peptide of 149 residues, the predicted value is 3.84 nm.

At higher q, the effect of concentration on the SAXS of OPN was significant (FIG. 5a), possibly due to self association in a solvent giving a negative second virial coefficient, but the SAXS of the OPN 1-149 was the same, within experimental error, at all the concentrations studied. The Kratky plot of a worm-like chain may exhibit a plateau at q² [symbol] r_{g}² [symbol] I(q)=2 if it is sufficiently long to give Gaussian chain statistics for distant segments of the primary structure. Neither OPN nor OPN 1-149 exhibited such a plateau region. Both peptides showed (FIG. 5b) a region in which I(q) varied as q⁻¹ which is a characteristic of a worm-like chain with short rod-like segments. However, the statistical nature of the polymer chain means that local backbone conformations need not persist in time, particularly since the Poly-L-proline-II (PP-II) conformation is not stabilised by any direct inter-residue H-bonding. Equation (24) gives b=1.9 nm for OPN, in agreement with the Kuhn length of 1.74±0.14 nm calculated here from the worm-like chain model. This length could correspond, for example, to an average of 5-6 residues temporarily arranged in a PP-II local helix. For OPN 1-149 equation (24) is inconsistent with the experimental radius of gyration. The lower chain stiffness of OPN 1-149 is possibly due to the higher proportion of Pro residues in this part of the sequence (10 out of the total of 13), each of which produces a sharp change of chain direction in the cis configuration, and of Gly residues (4 out of 4) which allow markedly more backbone chain flexibility than other residues because of the short side chain. Apart from Asp, the other residues are present in similar proportions in the two halves of OPN. It is possible, therefore that both OPN and OPN 1-149 contain similarly sized runs of local PP-II structure but in the latter the frequency of hinge residues allowing higher backbone flexibility is greater.

### Example 7

### Worm-Like Chain Model

Fully natively unfolded proteins and chemically denatured globular proteins show evidence of short range structure, predominantly of the PP-II type, but the root mean square radius of gyration, [symbol] r_{g}² [symbol] ^{1/2} of fully denatured proteins without disulphide bridges or secondary modifications shows a dependence on the number of residues, Nᵣₑₛ, given by (Kohn, J. E., Millett, I. S., Jacob, J., Zagrovic, B., Dillon, T. M., Cingel, N., Dothager, R. S., Seifert, S., Thiyagarajan, P., Sosnick, T. R., Hasan, M. Z., Pande, V. S., Ruczinski, I., Doniach, S. & Plaxco, K. W. (2004). Random-coil behavior and the dimensions of chemically unfolded proteins. Proceedings of the National Academy of Sciences of the United States of America 101, 12491-12496).

[symbol]

r_{g}² [symbol] =0.1927Nᵣₑₛ^{0.598} (nm) (16)

The exponent is close to the value of ⅗ expected of a homopolymer in a good solvent with excluded volume but the pre-factor is smaller than the peptide bond length.

For a Gaussian chain, [symbol] r_{g}² [symbol] =Lb/6 (L is the contour length, b is the Kuhn segment length of an equivalent freely jointed chain having the same root mean square end-to-end distance).

According to Benoit and Doty (Benoit, H. & Doty, P. (1954). Light Scattering from Non-Gaussian Chains. Journal of Physical Chemistry 57, 958-963), the corresponding relation for a worm-like chain is

$$〈 {r}_{g}^{2} 〉 = \frac{Lb}{6} [ 1 - \frac{3 b}{2 L} + \frac{3 {b}^{2}}{2 {L}^{2}} - \frac{3 {b}^{2}}{4 {L}^{3}} ( 1 - {e}^{- 2 L / b} ) ] \qquad ( 17 )$$

In the calculation of the contour length, a peptide bond length of 0.38 nm was assumed, corresponding to the planar all-trans conformation, though this ignores the effect of Pro peptide bonds which can introduce a kink in the cis configuration. In principle, evidence of short range structure can be obtained from SAXS or SANS measurements at higher values of q where deviations from the Gaussian statistics of random coils should become clearer.

The particle scattering factor of the worm-like chain of Kratky and Porod, as derived by Kholodenko (Kholodenko, A. L. (1993). Analytical Calculation of the Scattering Function for Polymers of Arbitrary Flexibility Using the Dirac Propagator. Macromolecules 26, 4179-4183) and validated by the Monte Carlo simulations of Pötschke et al. (Potschke, D., Hickl, P., Ballauff, M., Astrand, P. O. & Pedersen, J. S. (2000). Analysis of the conformation of worm-like chains by small-angle scattering: Monte-Carlo simulations in comparison to analytical theory. Macromolecular Theory and Simulations 9, 345-353) has been used to model the SAXS data. Experimental SAXS measurements on OPN and OPN 1-149, were obtained as a function of peptide concentration in the range 5-15 mg ml⁻¹. Radii of gyration and the intercept at q=0 were determined by a Guinier plot of ln(I) vs. q². Scattering curves were normalised by dividing by the Guinier intercept and weighted by q² to emphasize the low intensity features (Kratky plot). The normalised scattering at each concentration was then fitted to the structure factor taking account of the cross section of the chain.

### Example 8

### Nanocluster Model

For the OPN 1-149 nanocluster, a description of the peptide segment distribution around the core was considered which is more explicit than that provided by the core-shell model. The simplest block copolymer micelle model has been used in which a corona is formed by Gaussian chains attached by one end to a uniform spherical core. The nanoclusters were prepared either on the SAXS station and measured immediately or were prepared 2 days previously to allow the system to come to equilibrium. The sample was then stored at room temperature and remeasured 5 months later during a later allocation of beamtime. Preliminary experiments with nanoclusters prepared with the OPNmix sample established that the scattering was independent of concentration below a peptide concentration of 7 mg ml⁻¹. Accordingly, all measurements were made after dilution to 5 mg ml⁻¹ with the dilution buffer where, effectively, S(q)=1. The experimental SAXS results on the matured, equilibrium nanocluster were fitted to a polydisperse copolymer micelle model (Pedersen, J. S. & Gerstenberg, M. C. (1996). Scattering form factor of block copolymer micelles. Macromolecules 29, 1363-1365) using equation (23) with either a log-normal distribution or equation (15) in the ideal solution approximation. In addition, the particles were characterised by dynamic light scattering. The intensity averaged diffusion coefficient, D̅, was determined with a Malvern Zetasizer Nano instrument.

A polystyrene latex standard having a narrow size distribution and average hydrodynamic diameter of 20 nm was used as a standard. Inversion of the intensity autocorrelation function by means of the Multiple Narrow Modes algorithm in the instrument's software gave an intensity weighted size distribution which was used to test for polydispersity and the presence of much larger particles. If large particles were found they were removed by filtration of the nanocluster solution through a membrane of porosity 0.2 μm. The mean hydrodynamic radius ( r̅ₕ) was calculated from the diffusion coefficient using the Stokes-Einstein equation:

r̅ₕ=RT/6πηN_{A} D̅ (18)

is the viscosity of the aqueous medium. where η

Electrophoretic mobilities were measured by the phase analysis method in the disposable single-use cells supplied with the instrument. The zeta potential (ζ) was calculated from the electrophoretic mobility in a unit field (uₑ) using the Henry equation:

$$ζ = \frac{π η {u}_{e}}{f ( κ {\overline{r}}_{h} ) ɛ} \qquad ( 19 )$$

where ∈ is the dielectric constant and κ is the Debye-Hückel reciprocal length. The function ƒ(κ r̅ₕ) in equation (27) varies between the Hückel limit of ⅙ at small κrₕ and the Smoluchowski limit of ¼ at large κ r̅ₕ and at intermediate sizes is approximated by series expansions given by Henry, for example, at an ionic strength of 80 mM and a hydrodynamic radius of 22 nm, f(Krₕ)=1/4.9. No further correction for double layer relaxation was considered necessary for the low mobilities of the nanoclusters and relatively high ionic strength of the solution.

The results of the SAXS measurements on nanocluster sub-samples measured as a function of time after the addition of the urease are summarised in FIGS. 6a and 6b. The first two sub-samples were taken after 17 min when the pH was 6.82 and after 50 min when the pH was 6.87, but by the third sample the pH was essentially constant and close to 7.0. The Guinier estimates of the radii of gyration (FIG. 6a) and the complete SAXS (FIG. 6b) show the emergence of strongly scattering spherical particles from an initial state dominated by the scattering of a statistical polymer but after about 2 days the scattering profile was nearly constant. Studies with the recombinant phosphopeptides CK2-S, CK2-SS and CK2-S-6H also showed that their nanoclusters took 1-2 days to achieve the equilibrium size (FIGS. 12 and 13). According to the calculated degree of reaction of the phosphate centres and to the analysis of the correlation function given by the dynamic light scattering studies, the equilibrium state has a significant excess of free peptide, the scattering from which, although negligible at low q, has to be taken account of in modelling the SAXS at higher q.

Accordingly, equation (23) was used to recover the scattering of the nanocluster particles. The worm like chain representation of the free peptide was used with the assumption that the phosphate centres on the same peptide all react together to give either fully bound or fully free peptide so that α=α′. The counter assumption of free and independent phosphate centres would predict a much smaller fraction of fully free peptides and is inconsistent with the correlation function analysis of the dynamic light scattering results (see below). The weighted subtraction produced a scattering curve which is characteristic of spherical but polydisperse particles with a corona of statistical scattering elements. The Gaussian copolymer micelle model of Pedersen and Gerstenberg, together with the log-normal distribution function, produced a reasonably close representation of the scattering of the nanoclusters even though the OPN peptide chains in free solution are non-Gaussian.

### Example 9

### Electrophoretic Light Scattering by Nanoclusters

The size of the nanoclusters increased for some hours after the pH was raised through the action of the urease, but remained constant thereafter (FIG. 7). The equilibrium hydrodynamic radius of the β-casein 1-25 nanoclusters was 5.05 nm which is about 1 nm larger than the outer shell radius of a core-shell model derived from contrast variation SANS. When the casein phosphopeptide mixture was used to prepare nanoclusters by the mixing method and stored for 2 weeks at room temperature, the absorbance of the solution (A₆₀₀ₙₘ^{1cm}) fell from 0.017 to 0.003 in the first 5 d and remained constant thereafter. The hydrodynamic radius was affected by a very small proportion of particles with an average radius of about 150 nm. The large particles were evident in the correlation function and in the intensity weighted size distribution. After removing these by filtration through a 0.2 μm filter, the intensity averaged hydrodynamic radius was 6.05 nm, in good agreement with the (unfiltered) result obtained by the urea/urease method of 6.75 nm. The OPN 1-149 nanoclusters had a hydrodynamic radius of 21.9 nm after 2 days of equilibration which is comparable to the radius of gyration determined by SAXS. In the intensity weighted size distribution of unfiltered nanoclusters, there was a very small peak of much larger particles and another small peak, contributing 8.5% to the total scattered intensity, on the low side of the main nanocluster peak due to the free peptide. Because of the low scattering by the free peptide at optical wavelengths compared to the nanoclusters, the detection of a peptide peak is an indication that a substantial fraction of the peptide has not reacted to form nanoclusters and for this reason, whilst not wishing to be bound by theory, the inventors consider that all three phosphate centres react together to form the particle. Hence the fraction of free peptide is assumed to be the same as the fraction of unreacted phosphate centres, calculated from the chemical analysis to be about 62% at pH 7.0. Any precise comparison of the intensity averaged hydrodynamic radius from this technique with the so-called Z-averaged radius obtained by SAXS or SANS must take account of the different types of averages that the two methods generate, the different weightings given to heterogeneous particles at optical and X-ray frequencies and solvent drainage through the outer shell which affects the position of the plane of shear.

Hydrodynamic measurement gives a comparable estimate of the size of the nanoclusters to that from SAXS. The electrophoretic mobilities of the OPN 1-149 and CPP phosphopeptide nanoclusters were 1.4 and 1.0 μm s⁻¹ V⁻¹ cm. Using equation (27) and allowing for the differences of hydrodynamic radii and ionic strength these mobilities correspond to ζ-potentials, of −15.4 and −9.2 mV, respectively. Since the plane of shear of the casein nanoclusters is located about 1 nm from the outer surface of the nanoclusters, a distance which is comparable to the Debye-Hückel length, the surface charge density is effectively shielded by the double layer surrounding the nanoclusters. Nevertheless, the values reflect the larger net negative charge carried by the OPN 1-149 peptide compared to casein tryptic phosphopeptides.

### Example 10

### Microcalorimetry

The thermogram shown in FIG. 8 shows an almost perfectly smooth increase in specific heat with temperature, as expected for a polypeptide without a stable conformation. The change in specific heat capacity reflects the increased population of excited vibrational and rotational states with increase of temperature rather than any change in hydration of the backbone or side chains brought about by denaturation from a stable folded state. The result is also in accord with the low chemical shift dispersion in ¹H-NMR spectra of OPN.

### Example 11

### Cloning of the Coding Sequences for Native and Modified Bovine β-Casein

A method which routinely gives a yield of 10-100 mg of fully (or nearly fully) phosphorylated recombinant protein or peptide per litre of E. coli culture medium involving co-expression of a protein phosphokinase and a cognate protein (or peptide) substrate was used. Phosphotransferase casein kinase 2 (CK2) was used to phosphorylate β-casein or osteopontin at multiple sites, some of which are clustered together to form phosphate centres. Recombinant phosphorylation accurately reproduced the degree of phosphorylation that resulted from an optimised in vitro phosphorylation of the same (unphosphorylated) substrate with the chosen kinase. A full-length orf encoding β-casein (including the N-terminal signal sequence) was amplified by RT-PCR using appropriate oligonucleotide primers, from poly-A-enriched RNA, isolated from the mammary tissue of a lactating cow. Minor adjustments to base-sequence were subsequently made by site-directed mutagenesis, and verified by double-stranded sequencing, to ensure that the deduced translation product was the A² variant. Further sequence-modifications to yield an on of 633 bp were made by PCR-mutagenesis using suitable primers such that, at the 5′ end of the cDNA, the signal sequence was ablated and replaced by a translation-initiator codon in the context of an NdeI site. This methionine, in the deduced translation product, immediately preceded the N-terminal amino acid sequence of native mature secreted β-casein (RELEE—Table 14 (SEQ ID NO 209)). At the 3′ end of the cDNA, two sequence-variants were constructed. In one, the Stop codon of the wild-type base-sequence, terminating translation of the C-terminal sequence -PIIV•, was followed closely by a BamHI site to facilitate further sub-cloning operations, including ligation into the ampicillin-resistant expression vector pET21 (Novagen). In the other, the Stop codon was ablated and replaced by a BamHI site whose reading-frame allowed read-through to a 6-His-tag when BamHI was used, together with NdeI, for directional cloning of the 6-casein cDNA into pET21b. Thus, in this construct, the C-terminal sequence was extended to become -PIIVPRDPNSSSVDKLAAALEHHHHHH (SEQ ID NO 19). All PCR reactions were catalysed by the high-fidelity pfu polymerase (Stratagene) and PCR amplicons were initially cloned into the blunt-cloning propagation vector pPCR-Script (Stratagene). Sequence fidelity, orientation and reading-frame of the inserts in pPCR-Script were verified by double-stranded sequencing. All oligonucleotide primers and custom DNA sequencing were from MWG Biotech.

The generation of a phosphate centre through the action of CK2 required some adaptation to the G-CK recognition sequence in the native sequence. The canonical primary recognition sequence of the mammary gland G-CK is [S*,T*]X[E,D], (SEQ ID NO 210) where the residues in square brackets are alternatives and X is any residue, though with a preference for less bulky side chains. Most sites have S in preference to T and E in preference to D. The sequence [S*, T*][S*,T*][E,D] [E,D] (SEQ ID NO 211) containing two primary G-CK recognition sites therefore allows two neighbouring residues to be phosphorylated and a secondary recognition sequence of [S*,T*]X[pS,pT] allows phosphate centres to be formed from longer sequences of vicinal sites. The canonical primary recognition sequence of CK2 is similar: [S*,T*]XX[E,D] but D is considered better than E. A triplet of vicinal phosphorylated residues can therefore be formed by [S*,T*][S*,T*][S*,T*][E,D][E,D][E,D] (SEQ ID NO 212) and longer sequence of consecutive phosphorylated residues can be formed by exploiting a canonical secondary recognition site of [S*,T*]XX[pS, pT]. Site-directed mutagenesis of Serines and neighbouring residues within the phosphate centre of β-casein (to give CK2 smart- or CK2 smarter-sequences; see Table 16) was accomplished by PCR using the wild-type β-casein sequence as template, exploiting a unique SexAI restriction site immediately 5′ to codons for these residues. The mutating forward primers all incorporated this site towards their 5′ end; reverse primers spanned the unique StuI site downstream in the β-casein cDNA and incorporated a translationally silent diagnostic NspI site 5′ to it. Amplified sequences were blunt-cloned into pPCR-Script, propagated in the methylation-negative host SCS110 and diagnosed initially as SexAI-StuI inserts of the expected size. Qualifying candidates were submitted to double-stranded sequencing. A plasmid clone having the correct mutated sequence was used as the donor for transfer of a SexAI-StuI 541 bp cassette into the linearized plasmid resulting when the SexAI-StuI sequence was excised from the pET21-wild-type 6-His-tagged β-casein plasmid (see above). After ligation, transformants (in the methylation-negative host strain SCS110) were checked for the presence of the expected sizes of insert between SexAI-StuI and between NdeI-BamHI and for the presence of the extra diagnostic NspI site (giving rise to a 5-fragment digest with this restriction endonuclease). The manipulations generated two related pET21-derived expression plasmids, one encoding CK2-smart-β-casein-6-His; the other, CK2-smarter-β-casein-6-His.

Recombinant Constructs with an Enhanced Proportion of Phosphate Centre Sequence

CK2-smart- and CK2-smarter-β-casein constructs were produced that have the potential to yield a single phosphate centre as part of a readily isolated 28-mer tryptic peptide of MW approx. 3300 Da (see Table 14) from each molecule of recombinant modified β-casein of MW around 26700 Da. Thus when 100 mg of recombinant phosphoprotein was routinely recovered from the bacterial biomass generated from 1 litre of culture, the final yield of purified CK2-S or CK2-SS could not theoretically exceed ˜12 mg and was, in practice, typically around 3 mg. In an attempt to increase the final yield of phosphate centre peptides per litre of culture, two further coding sequences were constructed based on the successful CK2-smart design. In these constructs, the CK2-smart clustered Serine motif was present as a much greater proportion of the complete sequence for the primary recombinant translation product. The first of these (CK2-S-6H) has the deduced sequence shown in Table 14 and represented an attempt directly to express a phosphopeptide bearing a single phosphate centre within a short overall polypeptide sequence, effectively circumventing the requirement for digestion by trypsin and increasing the primary yield of phosphate centre peptide per unit weight of expressed protein by a factor of around 4.5. This construct was made by initially amplifying the first 96 bases of the cDNA for CK2-smart β-casein by PCR. The cDNA for CK2-smart β-casein served as a template. The design of the forward primer placed the initiator ATG in the context of an NdeI site and that of the reverse primer placed a BamHI site overlapping the GAG codon (bases 93-96) so as to enable in-frame translation to the 6-His-tag coding region of the expression vector pET21. When this amplicon was cloned into pET21b as an NdeI-BamHI fragment, the resulting on encoded a 53 residue polypeptide (MW 5945) having the sequence shown in Table 14. The second de novo construct (CK2-smart repeat; Table 14) was designed according to an alternative strategy, with multiple repeats of the CK2-S sequence separated by tryptic cleavage sites. To construct the coding sequence for this polypeptide, bases 4-96 of the cDNA for CK2-smart β-casein (i.e. omitting the initiator ATG codon) were amplified, by high-fidelity PCR. As above, the template for this was the cDNA for CK2-smart β-casein. Forward and reverse oligonucleotide primers were provided with blunt-cutter restriction endonuclease sites to allow for self-ligation of the amplicon molecules into which they would become incorporated, after cutting with these restriction enzymes. A SrfI site—GCCC/GGGC (blunt-cut site indicated by forward slash)—was placed immediately upstream of the first codon (AGA) of the forward primer. The sequence of the reverse oligonucleotide primer was such that, when read in the “sense” orientation, the bases GGAGG/CCT followed the triplet GAG encoding the last amino acid (Glu) of CK2-smart-β-casein 2-32. A StuI site occurs within these 8 bases; the position of its blunt-cut is shown by the forward slash. The population of amplicon molecules resulting from PCR amplification was then incubated with DNA ligase in the presence of the two blunt-cutting restriction enzymes SrfI and StuI which are able to remain catalytically active in the ligase buffer. The presence of the restriction enzymes assured that any head-to-head or tail-to-tail ligations (of the 102 bp amplicon generated after restriction with SrfI and StuI) were re-cleaved such that any ligated molecules surviving restriction were head-to-tail ligations. The DNA resulting from this ligation/restriction incubation was size-fractionated by agarose gel electrophoresis and molecules nominally between 500 and 1000 bp were excised, purified and blunt ligated into pPCR-Script. The size of any insert in each candidate recombinant plasmid was screened by digestion with NotI/PstI and its sequence was determined. It is inherent in these ligation/restriction/PCR/cloning techniques used here, that the population of recombinant DNA molecules generated comprised individual molecules having different numbers of repeats and with many having only one. In practice, time-constraints determined that the first qualifying candidate transformant with more than one repeat was developed further: in the event, this proved to have 3 repeats. It was excised from pPCR-Script with BamHI and NotI and ligated into pET29c which had been linearized by digestion with this same pair of restriction enzymes. In this plasmid context, the engineered construct could direct expression of a protein, CK2-smart repeat, whose deduced sequence (MW 14984) is shown in Table 14. It is clear that, compared with CK2-smart-β-casein, this protein affords a gain of approximately 5.3-fold in the yield of phosphate centre from a given mass of expressed protein. In principle, even greater yield advantages could be gained from constructs with more repeats or by truncation of the N-terminal sequence. Each of these “phosphate-centre-dense” constructs drove the abundant expression, in freely soluble form, of its encoded polypeptide. Co-expression with CK2α resulted in multiple-phosphorylation of their phosphate centres.

Cloning the CK2-Smart Phosphate Centre into a GST Chimera

The expression plasmid pET-42 (Novagen) was constructed with a multiple cloning site 3′ to the N-terminus of the coding sequence for GST, without intervening Stop codons, and flanked by tag sequences. PCR was used to prepare a family of amplicons across the phosphate centre sequence of CK2-smart-β-casein, cloned into pPCR-Script. The sequence of one of these was such that it could be excised from pPCR-Script with PstI+SacI and ligate it into pET42a after the plasmid had been linearized by digestion with the same pair of restriction enzymes. The deduced translation product from the resulting construct was a 365 amino acid chimera (MW 41215) in which the CK2-S phosphate centre peptide sequence was located, N-terminal to GST, within a fusion sequence flanked by oligo-His tags as well as other expression features intrinsic to pET42. This deduced sequence (GSTetc+CK2-S chimera) is shown in Table 16. A useful control fusion protein (MW 35448) could be expressed from the unmodified plasmid pET42b. The open reading frame of its coding sequence was the same as that of GSTetc+CK-2 etc except for the bases between the PstI and SacI sites: in pET42b they coded for 4 amino acids, whereas in the phosphate centre chimera, the inserted sequence was 53 amino acids in length.

Cloning of the Coding Sequence for Human Osteopontin A (hOPN-A)

Four splice variants of human osteopontin are known to exist, differing in the size of a translated exon close to the signal sequence at the N-terminus of the protein (Saitoh, Y., et al., 1995; Uniprot P10451). The primary translation products of OPN-A, OPN-B and OPN—C and OPN-D are, respectively 314, 300, 287 and 292 amino acids in length, with variant A having the greatest density of predicted CK2 phosphorylation sites. Its coding sequence was amplified by PCR using, as template, the IMAGE clone 3828885 (MRC Geneservice: www.hgmp.mrc.ac.uk), which was supplied in the propagation vector pDNR-LIB. The forward primer was designed to omit the signal peptide and to introduce into the amplified product a new initiator Met codon in the context of an NcoI site. This had the effect of changing the amino acid immediately C-terminal to the signal sequence (and, hence, the normal N-terminus of the mature protein) from Ile to Val. The reverse primer deleted the translation-termination codon and added a BamHI site in the correct reading-frame for translation through to the 6-His-tag of the expression vector pET21d (Novagen). The resulting amplicon was blunt-cloned into the propagation vector pPCR-Script and sequence-verified in that plasmid, before excision with NcoI+BamHI and directional cloning into pET21d as an insert of 897 bp. The expression product encoded in this pET21d-based construct is a C-terminally 6-His-tagged protein of 321 amino acid residues having the deduced sequence (MW 36228) shown in Table 14.

### Cloning of the Coding Sequence for Human CK2α

The plasmid hCK2α-pT7-7, a generous gift from the laboratory of Prof. Issinger (Grankowski, N. et al., 1991), contains the full-length coding sequence of human CK2α. This plasmid was propagated in the E. coli host strain XL1-Blue and excised the CK2α coding-sequence was excised from purified plasmid DNA as an NdeI-FspI fragment. This was then ligated into MCS 2 of the expression plasmid pACYC-Duet-1 (Novagen), linearized with NdeI-EcoRV. This strategy allowed directional cloning between the NdeI site and the blunt-ended EcoRV and FspI terminals. This cloning strategy resulted in inducible expression from the plasmid pACYC Duet-1 of the kinase catalytic subunit without attached fusion-tags. This plasmid expresses at relatively low copy number (˜11 in BL21 strains of E. coli) compared with plasmids of the pET family (copy number ˜40) and has 2 multiple cloning sites of which only one is used in this application. It carries a chloramphenicol-resistance gene. Plasmids based on pET vectors encoding phosphate centre constructs and the plasmid pACYC Duet-1-hCK2α were maintained, for propagation, in the XL1-Blue strain of E. coli in the presence of the appropriate selective antibiotic.

Co-Expression of Multiple Phosphorylation Candidates with CK2α

The non-secreting expression host strain E. coli—BL21star[DE3]—was dually transformed with a pET plasmid encoding the His-tagged or non-tagged phosphorylation candidate polypeptide and with pACYC Duet-1-hCK2α. Appropriate selective antibiotics ensured retention of the plasmids through subsequent handling of the transformed host. Inducible protein expression is driven from both pET and pACYC-Duet-1 vectors by the lac z gene. In practice, it was found that highest levels of expression of candidate phosphoproteins were obtained from overnight culture at 37° C. in the auto-inducing medium Overnight Express (Novagen). Bacterial cell pellets were harvested and washed by centrifugation and either stored at −20° C. after snap-freezing in liquid nitrogen, or used immediately for purification of intracellular recombinant protein.

### Purification of Recombinant (Phospho)Proteins and (Phospho)Peptides as His-Tagged Proteins

Bacterial cell pellets were lysed in 5 volumes of buffer (8M urea, 0.1M Na-phosphate, 10 mM Tris-HCl, 15 mM 2-mercaptoethanol, pH 8.0; containing a cocktail of protease inhibitors—Complete EDTA-free, from Roche diagnostics) by ultrasonication for 5×30 s at the maximum output of a 150 watt ultrasonic generator fitted with a 6 mm diameter probe. All but two of the recombinant 6-His-tagged phosphoproteins/phosphopeptides described in this study were expressed either in part or totally in insoluble form as inclusion granules. Supplementation of the lysis buffer with urea and its presence throughout subsequent stages of purification (see below) enabled these to become and to remain soluble. For the recombinant His-tagged phosphopeptides that were expressed in soluble form (CK2-S-6H and GSTetc+CK2-chimera; see Table 14) urea was absent during the purification. After centrifugation at 50000×g for 60 min, 6-His-tagged proteins were purified from the clear supernatants by metal chelate affinity (MCA) chromatography using columns (16 mm diam.×100 mm) packed with Ni-NTA resin (Qiagen). Following desorption of weakly bound proteins by inclusion of 10 mM imidazole in the wash-buffer, the proteins of interest were eluted in buffer containing 250 mM imidazole, observing the recommendations of the manufacturer. Protein was precipitated from this elution buffer by addition of 9 volumes of ice-cold acetone and the precipitate largely freed from residual buffer salts and urea by washing with ice-cold acetone:water (9:1) and dried in vacuo. Final purification was by preparative-scale reversed phase (RP-) HPLC using a column (25 mm diam. 150 mm length) packed with a polymeric beaded matrix (Polymer Labs; PLRP-S; 10 μm bead diameter; 300 Å pore size) equilibrated and run (10 ml/min) in a gradient of acetonitrile/0.1°)/0 formic acid in water/0.1% formic acid. Sample proteins for RP-HPLC were dissolved in water containing formic acid as required to promote solubility. Eluate was monitored for absorbance at 220 and 280 nm and was collected in 10-ml fractions. Those containing the protein of interest were pooled together and freeze-dried. The final purity of the 6-His-tagged CK2-smarter-β-casein (98%) revealed by RP-HPLC analysis was typical for all the recombinant proteins of this study.

### Non-Tagged Proteins

The CK2-smart repeat protein was concentrated by isoelectric precipitation at pH 4.5 from the centrifugal supernatant of bacterial ultrasonic lysates prepared, in the absence of urea, as above. After re-dissolving the precipitated protein in dilute formic acid, purification was achieved by preparative RP-HPLC as above. The purification of non-tagged recombinant bovine β-caseins (wild-type, CK2-smart- and CK2-smarter variants) used the same strategy but in this case urea was present throughout the bacterial lysis, centrifugation and isoelectric precipitation processes.

### Phosphopeptide Enrichment by Ba²⁺ Precipitation

The method for the selective precipitation of peptides containing multiple phosphoserine residues by barium salts was based on those previously published (W. Manson, W. D. Annan, Structure of a Phosphopeptide Derived from β-Casein. Archives of Biochemistry and Biophysics 145 (1971) 16-26.

E. G. Reynolds, P. F. Riley, N. J. Adamson, A Selective Precipitation Purification Procedure for Multiple Phosphoseryl-Containing Peptides and Methods for Their Identification. Analytical Biochemistry 217 (1994) 277-284). It was predicted that trypsin digestion would release one or more identical or closely related putative phosphate centre-containing phosphopeptides, susceptible to such precipitation, from those of our recombinant proteins that were constructed around bovine β-casein and also from both the CK2-smart repeat protein and the GSTetc+CK2-S chimera. Solutions of these proteins (at approximately 10 mg/ml; initially in the presence of urea if required) were extensively dialyzed against 50 mM Tris-HCl pH 8.0 before the addition of TLCK-treated trypsin to a final concentration of 0.2 mg/ml and incubation on an orbital shaker at 37° C. for 18 h. A second addition of trypsin was made to bring the concentration to 0.4 mg/ml, and incubation was continued for a further 2 h. Phosphopeptides were enriched from these trypsin digests by the following method, which was useful, also, for the concentration and further purification of the freely soluble tagged peptide CK2-S-6H, following its initial isolation by MCA chromatography from bacterial lysates as described above. First the pH of the sample, held at 0-4° C. throughout the procedure, was adjusted to 4.5 by the careful addition of HCl; any material becoming insoluble after standing for 30 min was removed by centrifugation. A solution (10% w/v) of BaCl₂ was added to the clear pH 4.5 supernatant to a final BaCl₂ concentration of 0.25% (w/v). Ethanol was then added, while stirring constantly, until a concentration of 50% (v/v) was attained. The precipitated phosphopeptide was collected by centrifugation and the pelleted material was allowed to dry in air. A final purification step of preparative RP-HPLC was applied to the resulting material using the conditions described above.

### Preparation of Calcium Phosphate Nanoclusters

Three recombinant phosphopeptides (CK2-S, CK2-SS and CK2-S-6H) were examined for their ability to sequester calcium phosphate in the form of nanoclusters. Their aligned sequences of are shown in Table 14 together with the N-terminal β-casein phosphopeptide [β-casein 4P (f1-25)] which was used as the template for producing these novel variations in phosphate centre sequences.

The CK2-S sequence, obtained by trypsinolysis of the β-casein-like precursor and isolation by barium precipitation and RP-chromatography, was used in most experiments. Three fractions of CK2-S were isolated from the leading edge, middle and trailing edge of the major peak in the final, preparative scale RP-HPLC purification of the barium phosphopeptide. So-called high affinity fractions of CK2-S and CK2-S-6H were recovered from the main peak in their respective hydroxyapatite chromatographic fractionations. Calcium phosphate nanoclusters are prepared by dissolving the peptide and the necessary salts at a pH low enough to prevent the formation of calcium phosphate complexes or precipitate. The formation of the nanoclusters is induced by raising the pH to a final value of 7.0 and the most elegant way of achieving this uses the urea/urease method as previously described. The salt concentrations used throughout were those for the Mg-free Buffer A and a peptide concentration of 5 mg·ml⁻¹ was employed for the CK2-S and CK2-SS peptides. These conditions provide very similar molar concentrations of peptide and salts to those used in earlier work with the native casein peptides β-casein 4P (f1-25) and α_{S1}-casein 4P 59-79. To provide a similar molar peptide concentration, the CK2-S-6H nanoclusters were prepared using a peptide concentration of 10 mg ml⁻¹ and diluted to 5 mg ml⁻¹ immediately prior to the SAXS measurement with the dilution buffer as previously described. The fresh nanocluster samples were prepared a few days before the allocated beamtime or at the SAXS station and, if there was sufficient material left over, stored at ambient temperature and re-measured once more after a period of up to 16 mo. The intervals between measurements depended on the timing of subsequent allocations of beamtime and on the satisfactory functioning of the synchrotron source and station.

### Recombinant β-Casein and Derivative Sequences: Phosphorylation by CK2

When the mature sequence of bovine A² β-casein (whether 6His-tagged or untagged) was co-expressed with CK2α and subsequently purified as described, the resulting protein was sub-stoichiometrically phosphorylated to only around 0.2 mol P/mol of casein compared to the 5P fully phosphorylated state of the protein expressed by the mammary gland. This result is consistent with the prediction of weak or absent recognition of the phosphate centre serines in bovine β-casein by CK2 (Meggio, F., Marin, O. and Pinna, L. A. (1994) Substrate specificity of protein kinase CK2. Cell. Mol. Biol. Res. 40, 401-409). In contrast, recombinant phosphorylation, by CK2, of the phosphate centre of human β-casein (data not shown) was accomplished in a way that closely replicated its heterogeneous phosphorylation state found in vivo, where phosphorylation is accomplished by the mammary gland Golgi kinase. This observation confirmed, with the twin-plasmid co-expression system described here, the result reported earlier by Thurmond et al. (Thurmond, J. M., Hards, R. G., Seipelt, C. T., Leonard, A. E., Hansson, L., Stromqvist, M., Bystrom, M., Enquist, K., Xu, B. C., Kopchick, J. J. and Mukerji, P. (1997) Expression and characterization of phosphorylated recombinant human beta-casein in Escherichia coli. Protein Expr. Purif. 10, 202-208), using recombinant technology involving a polycistronic expression construct encoding both α- and β-subunits comprising the CK2 holoenzyme. The common outcome from the different technologies used in the present work and by Thurmond and colleagues—that the physiological phosphorylation pattern of human β-casein was faithfully mimicked when the protein was co-expressed with CK2—strengthens the conclusion that differences in the detailed primary sequence and perhaps three-dimensional architecture between otherwise similar phosphate centres in different proteins can strongly influence their capacities as acceptor substrates for phosphorylation by protein kinases. As CK2 did not replicate the physiological phosphorylation of bovine β-casein, the substrate-suitability of the phosphate centre of the latter was enhanced by site-directed modification of its sequence to that shown as “CK2-smart-” in Table 14. The serines of this CK2-smart sequence are strongly-predicted phosphorylation targets of CK2, and when CK2-smart-β-casein was co-expressed, using the two-plasmid system, with CK2α, a high level of biosynthetic phosphorylation was attained (see below) suggesting partial attainment of a limit-stoichiometry of 4 mol P/mol protein. Further site-directed mutagenic modification of the CK2-smart sequence was undertaken to yield a possibly CK2-smarter tryptic peptide termed “CK2-SS” as shown in Table 14. When CK2-smarter-β-casein was co-expressed in E. coli with CK2α, analysis of its resulting biosynthetic phosphorylation (see below) indicated the occupation, in at least some of the molecules, of all 4 serines in the engineered phosphate centre. In all cases tested, these levels of biosynthetic phosphorylation were similar to those that were achieved when recombinantly-produced non-phosphorylated versions of these proteins (i.e. expressed from hosts not also harbouring the CK2α expression plasmid) were incubated in vitro with CK2α under optimal conditions for phosphokinase activity (data not shown).

### Trypsin Release of Phosphopeptides

Tryptic digestion of biosynthetically phosphorylated CK2-smart- and CK2-smarter-β-casein was predicted, by analogy with the well-characterized digestion of native β-casein 5P, to release a multiply-phosphorylated peptide (see trypsin cleavage peptides identified in Table 14). This prediction was tested by fractionation of a tryptic digests with Ba²⁺ and ethanol for the selective precipitation of phosphopeptides as described above. The principal peptide component from the resulting precipitates was purified, then characterized by analytical RP- and strong anion exchange HPLC, using the phosphopeptide β-casein 4P 1-25 as a reference and positive control. Chromatographic properties of the putative phosphopeptides from CK2-smart- and CK2-smarter-β-casein were similar to those of the authentic positive control phosphopeptide.

Native β-Casein 4P (f1-25)

This peptide was used to see whether the 4 known and unique fully phosphorylated sites in this pure and well-characterised material could be located by ion trap methods alone. In MS¹ mode two peaks at m/z˜1562 and 1042 were seen corresponding to the 2+ and 3+ ionisation states, along with very many other fragments. In the MS² spectrum of the 1562 peak there were 4 prominent peaks at m/z˜1513, 1464, 1414 and 1365 derived by P-loss from the 4P parent 2+ ion at 1562. Additional prominent peaks were formed by the further loss of up to 3 water molecules. This confirms that only 4 of the 5 Serine residues are phosphorylated but not which ones. The 1365 peak was selected for further MS³ analysis as all 4 phosphorylated residues have been converted to the Dehydroalanine form. In the MS³ spectrum, peaks at m/z values of 1608, 1677, 1790, 1859, 1927 and 1997 correspond to the b₁₄, b₁₅, b₁₆, b₁₇, b₁₈ and b₁₉ ions respectively. The occurrence and m/z of the ions b₁₅, b₁₇, b₁₈ and b₁₉ localises the phosphate groups to S-15, S-16, S-17 and S-19 and confirms that S-22 is unphosphorylated.

### CK2-S

The major features of an otherwise quite complex infusion-MS' spectrum were peaks at m/z˜1668 (2+) and 1112 (3+) predicting a parent peptide of 3336 Da. Among the potential tryptic peptides derived from the recombinant protein sequence was a peptide with two missed tryptic cleavage sights comprising residues 3-30 of CK2-smart β-casein (Table 16) having a calculated mass of 3333 Da in its 3P form. There are 4 potential sites of phosphorylation, allowing 4 potential positional isomers. MS² analysis of the 1668 peak was carried out and it was readily confirmed that there were prominent peaks corresponding to 1, 2 and 3 neutral losses of phosphate and in each case up to two further losses of a water molecule from the parent ion. A search was made for the b- and y-ions and their derivatives due to neutral loss of phosphate and water molecules using the predictions of this candidate sequence. Among the most prominent 40 peaks were the further derivatives or original fragment ions corresponding to b₇, b₁₄, b₁₆, b₁₇, b₁₈ and b₁₉ and y₁₃, y₁₅, y₁₇ and y₁₈. In the MS² spectrum of the 1112 peak it was possible to identify all the b ions predicted for the sequence up to residue 12. Thus the candidate sequence was confirmed. Moreover, there were prominent peaks corresponding to the loss of three phosphate molecules from both the predicted b₁₆ and b₁₈ ions, confirming that at least the major phospho-isomer is phosphorylated at residues 13, 14 and 15. MS² analysis of the 1112 peak also demonstrated neutral losses of up to three phosphate moieties from the y₂₋₂₂ ion. The fully dephosphorylated member of this series was further analysed by MS³ and sufficient of its sequence identified to confirm phosphorylation at S-13, S-14 and S-15 but not S-19. Nevertheless, some evidence was found by MS² analysis of other 2+ peaks in the MS¹ spectrum that not all the species present are triply phosphorylated. Thus a peak at m/z˜998 showed losses of only two phosphate moieties whereas a peak at m/z˜1043 showed neutral losses of up to 4 phosphates. Analysis of the same tryptic peptide by tandem LC-MS using a polymeric reversed-phase PLRP-S column gave only one significant peptide (UV absorbance) peak but the MS¹ and MS² spectra recorded during the passage of this peak indicated that there was significant heterogeneity, with the main fraction comprising the fully phosphorylated 4P peptide. In the leading edge of the peak, smaller signals arose from two isomers of the 3P peptide either unphosphorylated at S-13 or S-19 and a further peptide was detected corresponding to a 4P form with the additional sequence -IEK• at the C-terminus due to a missed tryptic cleavage. The relative proportions of the different peptides and phosphoforms could not be determined quantitatively from this analysis.

### CK2-SS

This phosphopeptide yielded a qualitatively similar complex infusion-MS spectrum to that described above for the phosphopeptide CK2-S, with major peaks at m/z 1668 (2+) and 1112 (3-F). MS² analysis of the 1668 peak revealed a neutral phosphate loss series, and accompanying water loss, for 3 phosphorylated serine residues. This peptide is an isomer of CK2-S and their deduced sequences differ only in the single transposition of an aspartate and a serine residue. Having established their equivalence in terms of mass and phosphorylation density, suggesting that this peptide was no better-recognized by CK2α as a phosphorylation substrate than CK2-S, no further MS or LC-MS analysis was carried out on the CK2-SS variant.

### CK2 Smart Repeat

Deconvolution of the mass spectrum of this protein gave rise to a family of predicted molecular masses corresponding exactly to values expected for a range of phosphorylation states (from 0-11 mol P/mol of protein). The 0P-6P phosphoforms were present in small (<10% of total) and approximately equal relative amounts. The 9P phosphoform was the most abundant, closely followed by the 8P and 10P phosphoforms, then by 7P and 11 P phosphoforms at approximately half their concentration.

### CK2-S-6H

This phosphopeptide was analyzed in some detail by tandem LC-MS. MS scans were recorded and analyte samples were collected at sampling points numbered sequentially 1-6, encompassing all absorbance peaks. All scans between sampling points 1 and 6 showed prominent mass signals corresponding to the 5+, 6+, 7+ and 8+ ionisation states. In addition, at sampling points 1 and 2, recognisable signals corresponding to the 9+ state were revealed, and at sampling points 3-6, signals corresponding to the 4+ state. Biomass calculations from these MS scans gave masses of 5948.0, 6030.1, 6107.3, 6187.7, 6267.7 and 6348.1 Da, yielding the conclusion that analytes sampled at points 1-6 comprised respectively 0P, 1P, 2P, 3P, 4P and an approximately equal mixture of 4P and 5P phosphoforms of the CK2-S-6H sequence (shown in Table 14). Integrated areas under the absorbance peaks having their maxima at sampling points 1-6, expressed as a percentage of the total were 2, 2, 10, 26, 54 and 6%, demonstrating that most of the peptide is in the 4P form. Assuming an equal mixture of 4P and 5P phosphoforms in fraction 6, the weight average degree of phosphorylation is 3.43. Direct infusion of a tryptic digest of the CK2-S-6H peptide into the electrospray ionizer allowed identification of a number of phosphopeptides in the MS' spectrum. The most prominent peaks corresponded to tryptic peptides containing the phosphate centre sequence, -DDSSSDDDSDDD- (SEQ ID NO 20) with 2, 3 or 4 phosphorylated residues. Other prominent peaks were identified as arising from the unphosphorylated peptides KIEDPNSSSVDK (SEQ ID NO 21) and IEDPNSSDK (SEQ ID NO 22). Taken together, these findings confirm that the primary phosphate centre provides the main location of acceptor-sites for the kinase during recombinant co-expression with this peptide. Nevertheless, when the peptide was further fractionated by hydroxyapatite chromatography and the individual fractions collected and analysed by tandem HPLC-MS, a small proportion of the peptide was in the 6-P form, which is only possible if the minor cluster of sites of phosphorylation can have up to two of its three serines phosphorylated

### GSTetc+CK2-S Chimaera

Only very limited MS data were obtained for this protein, confirming 70% of its deduced sequence from a LC-MS analysis (using a conventional C18 PepMap nano-column) of its tryptic digest. This analysis confirmed the presence of tryptic peptides, with up to 3 phosphorylations, covering the inserted CK2-smart sequence and overlapping between it and the GST fusion-partner molecule.

hOPN-A

This protein proved to have a complex MS signature which could be reconciled with the deduced sequence only in terms of the loss of Histidine or of a Methionine (presumably from the C- and N-terminus respectively) the latter accompanied by oxidation of a Histidine. It is most likely that these modifications occurred during electrospray ionization of the protein in the mass spectrometer. Nevertheless, several neutral phosphate loss series of at least 4 phosphates were clearly found arising from these mass-deleted parent ions. The protein contains six target sequences for CK2. In Table 14 partial sequences of the N-terminal region only are shown for β-casein and its CK2-smart- and CK2-smarter-derivatives. Sequences of the other polypeptides are shown in full, and the first 5 sequences are aligned with respect to residues flanking their respective phosphate centres. Actual sites of phosphorylation by the Golgi kinase or strongly predicted phosphorylation sites for CK2 and sequences preserved from the native β-casein are in bold type. The predominant tryptic peptide containing the phosphate centre sequence from each of the β-casein-related proteins is underlined; the analogous phosphate centre peptide in the GSTetc+CK2-S chimera is double-underlined.

### Example of Use of Calcium Phosphate Sequestration for Improved Formulation of Substitutes for Biological Fluids

The use of an OPN 1-149 peptide offers improved performance in terms of terminal steam sterilisation, low immunogenicity and higher sequestration over substitute biofluidformulations disclosed previously. Excess phosphopeptides present in such a solution are advantageous, as they will act to (i) suppress calcium phosphate precipitation during sterilization and storage of the plasma substitute and (ii) suppress ectopic calcification during use. The provision of calcium in the form of a thermodynamically stable solution of calcium phosphate nanoclusters is designed to buffer the free ion concentrations of calcium and phosphate and the pH under the circumstances where acidosis develops or where the normal physiological mechanisms result in the removal of the calcium and phosphate ions. In particular embodiments a phosphoprotein or phosphopeptide normally present in plasma, such as OPN, in particular OPN 1-149 or suitable fragments thereof fetuin A, SPP-24 or matrix Gla protein, can be utilised to form nanoclusters in substitute biofluid formulations with such nanoclusters having a low immunogenicity.

In one example a plasma substitute formulation includes an optimum level of phosphate and a phosphopeptide or phosphoprotein containing at least one phosphate centre. The solution can have a pH of 7.3-7.5, an osmolality of 280-310 mM, and an oncotic pressure of 20-30 mm Hg. Osmolality is determined primarily by the electrolytes, as well as by the oncotic agent, the phosphopeptide and the optional ingredient glucose (preferably 0-125 mM). Agents, such as dextran (0-100 gm/l) and polyethylene glycol (0-25 gm/l) may be added to give the required oncotic pressure. Optionally, anti-oxidant or free radical scavengers, such as mannitol (0-20 gm/l), glutathione (0-4 gm/l), ascorbic acid (0-0.3 gm/l) and vitamin E (0-100 IU/l) may be provided. The phosphopeptide may be present in the range 0.5-2.0 mM, total Ca⁺⁺ in an amount ranging from about 0.5 to 4.0 mM; total Cl⁻ in an amount ranging from 70 to 160 mM; total Mg⁺⁺ in an amount ranging from 0 to 10 mM; total K⁺ in an amount ranging from 0 to 5 mM; total phosphate in an amount ranging from 5-15 mM and, optionally, a simple hexose sugar from 2 to 50 mM; wherein said solution is terminally heat sterilized. NaHCO₃ can be added as a commercially-available sterile 1 M solution to the sterilized solution immediately before use. Generally, 5 ml of a 1 M NaHCO₃ solution can be added per litre, but more may be added.

Methods of preparing the solution will closely follow the methods previously disclosed for the preparation of casein nanoclusters, namely the simple mixing method or the urea/urease method as described above and as would be understood in the art. To prepare the solution, all the ingredients except for the phosphate salts can be dissolved in a volume of water comprising not more than 90% of the final volume. The pH of the solution is then raised to pH 7.3-7.5 by the addition of aliquots of stock solutions of two or more of the mono-, di- or tri-sodium phosphates or of NaOH. The additions are made slowly under conditions of rapid mixing to avoid the local excess concentrations that can result in the irreversible precipitation of calcium phosphate. When the final pH and total phosphate additions have been made, the solution is made up to volume and heat sterilised by autoclaving at 120 C for 15 min.

In an alternative method, all the ingredients are added to 90% of the final volume of water to give a pH of 5.0 and sufficient urea is added to raise the pH to 7.3-7.5 by means of 2-20 units of urease. The urease is dissolved in 1 ml of water and may be placed into a dialysis sack to prevent it entering the plasma substitute solution. The pH is monitored with gentle stirring for 24 hr and additional urea added to the plasma substitute solution as needed to achieve the final pH, following which the dialysis sac is removed and the solution sterilized.

### Example Formulation of a Stable Human Plasma Substitute by the Urea/Urease Method

Using 40 mg of OPNmix as the phosphopeptide, 10 ml of an artificial plasma which included nanoclusters was prepared by the urea/urease method using the following additions of stock solutions: 270 μl of CaCl₂ (100 mM), 100 μl of MgCl₂ (100 mM), 1380 μl of NaCl (1-M), 250 μl of KCl (100 mM), 100 μl of NaN₃ (150 mM), 200 μl of NaH₂PO₄ (100 mM), 572 μl of NaHCO₃ (100 mM), 5000 μl of glucose (100 mM) and 30 μl urea (1-M). The pH was recorded as 6.642 and this was reduced to 5.267 by the addition of 60 μl HCl (1-M) and 2038 μl of H₂O added to bring the final volume to 10 ml. At pH 5.267, the solution was calculated to be undersaturated with respect to ACP and DCPD. A stock solution of urease (Sigma Type C-3 from Jack bean) at a concentration of 10 mg ml⁻¹ was made up and 40 μl placed in a visking dialysis tube which was sealed and placed in the artificial plasma solution. After 92 min the pH had risen to 6.960 and over the following 3 h three additions, each of 1 μl of the stock urea solution were made to bring the pH tp a final stable value of 7.367 at which point the dialysis sack was removed and the clear solution stored at ambient temperature.

### Example Formulation of a Stable Human Plasma Substitute by the Simple Mixing Method

Using 20 mg of OPNmix as the phosphopeptide, 10 ml of an artificial plasma which included nanoclusters was prepared by the simple mixing method using the following volumes of stock solutions: 270 μl of CaCl₂ (100 mM), 100 μl of MgCl₂ (100 mM), 1380 μl of NaCl (1-M), 250 μl of KCl (100 mM), 200 μl of Na₂HPO₄ (100 mM), 400 μl of OPNmix (50 mg ml⁻¹), 5000 μl of glucose (100 mM), 572 μl of NaHCO₃ (100 mM). After mixing, the pH was 6.760 and this was raised slowly with stirring to 7.48 with 110 μl of 1-M NaOH and the volume made up to 10 ml with H₂O. The composition and the calculated properties of the solution are shown in Table 15. The solution was optically clear and remained so after steam sterilization at 126 C and 1.5 bar for 20 min. An aliquot of the sterilized artificial plasma was then freeze dried and reconstituted in the original volume of distilled water. A slight gelatinous precipitate appeared as the freeze dried powder dissolved, but this disappeared within a few minutes to give a clear solution with a pH of 8.045. The pH was reduced to 7.139 by addition of 1-M HCl and after 45 min the pH of the optically clear solution was stable at 7.190. For the shelf life determination, NaN₃ was added to the unsterilized and reconstituted samples to a final concentration of 1.5 mM. The three samples prepared by the mixing method were stored at ambient temperature for three weeks and examined by dynamic light scattering. Dynamic light scattering measurements used a Dynapro 801 TC instrument from Protein Solutions Ltd. Prior to the measurement, the sample was filtered through a Whatman Anotop 10 filter with a pore size of 0.2 μm. Measurements were made at 25 C and the hydrodynamic radius was calculated from the intensity averaged diffusion coefficient using the Stokes-Einstein equation. Correlation functions were inverted to give an intensity weighted distribution of hydrodynamic radii using the singular value decomposition method of Laplace transformation implemented in the DynaLS program of Alango Ltd. Neither the unsterilized sample nor the reconstituted one scattered enough light to give a stable measurement of particle size. The sterilized sample, however, showed enough scattering for analysis, revealing an intensity average hydrodynamic size of 4.53 nm. The normalised intensity distribution showed a strong peak of the size expected for the free peptide and a smaller peak of nanoparticles with a modal radius of 36.2 nm. The nanoparticle size formed by this mixture of phosphopeptides is nearly twice as large as the nanoclusters formed by OPN 1-149 with Mg-free and carbonate-free Buffer A. Notwithstanding this, the artificial plasma has withstood terminal heat sterilisation and is readily reconstituted from a freeze dried state as would be expected if the nanoparticles are also calcium phosphate nanoclusters.

TABLE 15
Calculated properties of the artificial plasma at pH 7.48

| Component | Concentration, | Component or | Concentration, |
|---|---|---|---|
| or property | mM | property | mM |
| Total Ca | 2.70 | Free Ca²⁺ | 1.25 |
| Peptide-bound Ca | 0.11 | Nanocluster Ca | 0.58 |
| Total Mg | 1.00 | Free Mg²⁺ | 0.60 |
| Peptide-bound Mg | 0.05 | Nanocluster Mg | 0.04 |
| Total Pᵢ | 2.00 | Nanocluster Pᵢ | 0.29 |
| Total Na | 147.50 | Total K | 2.50 |
| Total Cl | 157.50 | Total carbonate | 5.72 |
| OPN 1-149 | 0.05 | Glucose | 50.0 |
| Ionic strength | 152.50 | Osmolarity | 270.2 |

### Example of Use of Fetuin a in Formation of Nanoclusters

Fetuin A was purchased from Sigma Aldrich as product no. F3004. The purity of the Sigma fetuin A was found to be about 80-90% as judged by SDS PAGE with significant impurities of both higher and lower molecular weight. Although attempts were made to make nanoclusters with this material using the Mg-free Buffer A and the urea/urease method at a protein concentration of 66.4 mg ml⁻¹ (1.6 mM assuming a molecular weight of 36,353 Da), a fraction of the product precipitated making it impossible to determine the size of the fetuin A nanoclusters. The inventors determined the precipitation problem was much reduced at lower fetuin A concentrations so an approach was adopted in which mixed nanoclusters were prepared with casein phosphopeptides and a variable concentration of the fetuin A to recover the size of the fetuin nanoclusters by an extrapolation procedure from lower concentrations. The casein phosphopeptide mixture was supplied by Arla Ltd, Denmark as product Lacprodan 2090 (Na salt). Nanoclusters prepared with the casein phosphopeptide mixture material have been described previously (Holt C, Sørensen E S & Clegg R A (2009) Role of calcium phosphate nanoclusters in the control of calcification. FEBS Journal 276, 2308-2323, doi: 10.1111/j.1742-4658.2009.06958.x and Little E M & Holt C (2004) An equilibrium thermodynamic model of the sequestration of calcium phosphate by casein phosphopeptides. European Biophysics Journal with Biophysics Letters 33, 435-447, doi: 10.1007/s00249-003-0376-x). Samples were prepared of Lacprodan 2090, dissolved at a fixed concentration of 10 mg ml⁻¹ in Mg-free Buffer A at pH 5, together with 0, 1, 2, 4, 8 or 16 mg ml⁻¹ of the fetuin A respectively. The pH was raised using 30 mM urea and 10 units urease per ml to give a final pH of 7.65±0.15 within 1 day. The samples with 0 and 1 mg ml⁻¹ fetuin A; however, reached a higher pH which was reduced by addition of small aliquots of 1-M HCl to match the pH of the other samples and a further day allowed for equilibration before determining the hydrodynamic size. The intensity weighted normalised distribution curves calculated by Laplace transformation of the correlation functions of the 6 samples, offset vertically for clarity (FIG. 18a) indicate a nanocluster peak of about 6 nm in accordance with previous work, but as the fetuin A concentration increased, a second peak became increasingly important at larger size. Nevertheless, at these lower fetuin concentrations the aggregated material did not interfere with the determination of the modal value of the nanocluster peak. Although difficult to estimate exactly, on the basis of scattering by homogeneous spherical particles, the weight fraction of aggregated material in these experiments was calculated to be less than 1% in all cases. The effect of increasing the fetuin concentration was generally to decrease the hydrodynamic size (FIG. 18b) although the initial effect was to produce a small increase. To calculate the hydrodynamic size of fetuin A nanoclusters an extrapolation to infinite concentration was made in which the hydrodynamic radius was plotted against the reciprocal of the fetuin A concentration (FIG. 18c) from which it was found that the fetuin A nanoclusters had a hydrodynamic radius of 5.8 nm.

It had been expected that the spiking of the nanocluster with fetuin A would increase the hydrodynamic radius. Fetuin A is thought to have two globular cystatin domains in the N-terminal half of the sequence and an unfolded C-terminal half containing a single phosphate centre. The incorporation of the fetuin A in a nanocluster might be expected to increase the shell thickness, as was indeed observed at the lowest fetuin A concentration. However the core radius also depends on the sequestration free energy of the shell as a whole and if the binding of fetuin A makes this less negative then the core radius will be reduced. The modest decrease in hydrodynamic radius produced by the incorporation of the fetuin suggests that both effects might be close to cancelling each other out.

The fetuin nanoclusters provide another example of the formation of nanoclusters by secreted phosphoproteins and moreover, the first example of nanocluster formation by a non-SCPP, the first example of a nanocluster formed by a non-casein whole protein and the first example of a nanocluster where part of the sequestering phosphoprotein is predicted to have a stable globular conformation. Because fetuin A is a normal component of plasma, nanoclusters prepared with fetuin A or phosphopeptides from this sequence are expected to have low immunogenicity which is particularly useful for biomedical applications such as drug delivery, vaccine adjuvants and artificial biofluids.

### Stabilization of Urine-Like Solutions Against Precipitation of Calcium Oxalate

The prevention or inhibition of crystal growth in urine is desirable because approximately 5% of the population of developed countries suffer from urolithiasis. The inventors consider nanocluster formation, with certain types of phosphopeptides, for example OPN 1-149 or OPNmix, may be useful in the treatment of this condition. Other stabilizing proteins and peptides could be used and would be obvious to someone skilled in the art. Urine typically has a pH around 6 and comprises a homogeneous solution with or without crystals of various salts, most commonly calcium oxalates, uric acid and urates and calcium phosphates and at high pH magnesium ammonium phosphate. Previous studies have suggested the total salt concentrations in human urines from healthy and various classes of stone-forming subjects (Robertson W, Peacock M & Nordin BEC (1968) Activity products in stone-forming and non-stone-forming urine. Clinical Science 34, 579-594). It is postulated that precipitated phases form particles that are too small to be detected by the usual methods of urine analysis.

The effect of macromolecules on the ion equilibria of urine has never been determined other than through the well-known reductions in the rates of crystal growth. Quantitative determination of the levels of OPN peptides in urine, particularly molar concentrations, are subject to a number of systematic errors and uncertainties (Kon S, Maeda M, Segawa T, Hagiwara Y, Horikoshi Y, Chikuma S, Tanaka K, Rashid M M, Inobe M, Chambers A F, et al. (2000) Antibodies to different peptides in osteopontin reveal complexities in the various secreted forms. Journal of Cellular Biochemistry 77, 487-498 and Thurgood L A, Grover P K & Ryall R L (2006) A problem protein: Unexpected analytical irregularities in the measurement of urinary osteopontin. In (Evan A P, Lingeman J E & Williams J C, eds), pp. 196-199, Indianapolis, Ind.) and widely differing results have been reported.

An example artificial urine was used which closely approximates the average salt composition of urine from the control group of 60 healthy male subjects in the work of Robertson et al. It was prepared by the simple mixing method from the following stock solutions: CaCl₂ (100 mM), MgCl2 (100 mM), NaCl (1-M), KCl (1-M), (NH4)2SO4 (100 mM), NaH2PO4 (1000 mM), H3-citrate (100 mM), H2-oxalate (20 mM), OPNmix (50 mg ml-1), Na2-urate (4 mM) and HCl (1-M). After mixing in the order given in Table 16, but before the addition of the HCl, the pH was 5.3 and after the addition of the HCl was 5.0. The composition and the calculated properties of the solution are shown in Table 16. The sodium urate stock was prepared by titating uric acid to pH 8. In the absence of the osteopontin, a precipitate of uric acid formed which could be resolubilised on warming the solution. If the urate concentration was reduced to 0.5 mM or less, the artificial urine was stable at room temperature at pH 5. At a urate concentration of 1.5 mM, precipitation could be prevented by the addition of the osteopontin stock solution. In two other formulations with either 1.0 or 0.1 mg ml⁻¹ OPNmix, no precipitation of uric acid was observed.

Aliquots of the standard artificial urine were adjusted in pH by addition of 1-M NaOH with good stirring to give samples in the approximate pH range 5-8 and steam sterilized at 126 C and 1.5 bar for 20 min. No precipitation occurred and the pH of the samples were 5.048, 5.387, 5.995, 6.385, 7.380, 7.870, 8.000 and 8.237. Solutions continued to be stable when stored at room temperature for 1 month before being examined by dynamic light scattering.

TABLE 16
Production of a stable artificial urine at pH 5.0

| | | μl added | | Concentration, |
|---|---|---|---|---|
| Stock | mM | per ml | Component | mM |
| CaCl₂ | 1000 | 43.4 | Ca | 4.34 |
| MgCl₂ | 100 | 31.8 | Mg | 3.18 |
| NaCl | 1000 | 96.0 | Na | 118.00 |
| KCl | 1000 | 27.4 | K | 42.00 |
| (NH₄)₂SO₄ | 100 | 85.0 | NH₃ | 17.00 |
| K₂SO₄ | 100 | 73.0 | SO₄ | 15.80 |
| OPN | 500¹ | 50.0 | OPN | 2.50¹ |
| NaH₂PO₄ | 1000 | 20.5 | Pᵢ | 20.50 |
| Citric acid | 100 | 23.0 | Citrate | 2.30 |
| Oxalic acid | 20 | 7.45 | Oxalate | 0.15 |
| Sodium Urate² | 4 | 375.0 | Urate | 1.50 |
| HCl | 1000 | 20.0 | Cl | 158.44 |
| Water | | 147.45 | H | 68.05 |
| Total volume | | 1000.00 | pH | 5.00 |

¹mg ml⁻¹

### Discussion of Core-Shell Structure

From the determinations of the inventors, the radius of gyration of the OPN peptide on the core surface was about a third of its value in free solution. Without wishing to be bound by theory, the inventors consider the peptide is attached to the surface at several points such as the three phosphate centres. This assumption is also consistent with the mole fraction of free peptide, the composition of the nanoclusters and the calculated mole fraction of reacted phosphate centres. Compared to previous casein nanoclusters, the core calcium phosphate of the OPN nanocluster was more basic, corresponding to the empirical chemical formula of a tri-calcium phosphate, but the molar ratios of Ca or Pᵢ to PCs were calculated to be the same. According to the SAXS analysis, the core of OPN 1-149 nanoclusters were nearly about four times as large as those prepared with previous casein phosphopeptides and approximately twice as large as those prepared with the recombinant CK2-S, CK2-SS and CK2-S-6H peptides. Based on the results of the inventors studies, there is provided a means to increase the size of nanoclusters by modulating factors such as the sequestering power of the phosphate centres, modifying the core surface area per phosphate centre and the hydration and degree of longer-range order in the calcium phosphate. As the core surface area per phosphate centre in the OPN 1-149 nanoclusters was found to be 0.25 nm², which is about a quarter of that calculated for the nanoclusters made with β-casein 1-25, this alone could account for the difference in size of the OPN nanoclusters over the previously known casein nanoclusters

Various modifications may be made to the invention herein described without departing from the scope thereof.

1-44. (canceled)

45. A thermodynamically stable calcium phosphate nanocluster wherein said nanocluster comprises a phosphopeptide or phosphoprotein, the phosphopeptide or phosphoprotein being selected from; a) a recombinantly expressed phosphopeptide or phosphoprotein wherein said recombinantly expressed phosphopeptide; i) includes a phosphate centre modified such that at least one of a phosphorylated residue and an acidic residue or combinations of these residues is increased within the phosphate centre such that the phosphate centre has increased calcium phosphate sequestering power, or ii) is modified such that the modified recombinantly expressed phosphopeptide includes an increased number of discrete phosphate centres in comparison to a non-modified version of the recombinantly expressed phosphopeptide, or iii) is modified such that the modified recombinant phosphopeptide has increased calcium phosphate sequestering power over a non-modified version of the recombinantly expressed phosphopeptide, preferably by alteration of the number and/or type and/or phosphorylation of the amino acid residues or spacing of particular amino acid residues within a phosphate centre, or removing amino acid sequences which promote the conversion of amorphous calcium phosphate into a more crystalline phase such as apatite, b) a calcium binding phosphoprotein/phosphopeptide, or a variant or a fragment thereof wherein the phosphopeptide or phosphoprotein does not include an individual casein or mixture of caseins or enzymatic digests of an individual casein or mixture of caseins, provided that at least one phosphopeptide or phosphoprotein optionally may be an individual casein, a mixture of caseins or fragment thereof wherein glutamate residues in a phosphate centre of the casein are substituted with aspartate residues, or c) a combination of a) and b).

46. A thermodynamically stable calcium phosphate nanocluster as claimed in claim 45 wherein the phosphopeptide or phosphoprotein comprises the amino acid sequence of at least one member selected from the group consisting of fetuin A (SEQ ID NO 10), proline-rich basic phosphoprotein 4 (SEQ ID NO 11), matrix GIa protein (SEQ ID NO 12), secreted phosphoprotein 24 (SEQ ID NO 13), osteopontin (SEQ ID NO 15), integrin binding sialophosphoprotein (SEQ ID NO 16), matrix extracellular bone phosphoglycoprotein (SEQ ID NO 17), dentin matrix acidic phosphoprotein 1 (SEQ ID NO 18) or variants or fragments thereof.

47. A thermodynamically stable calcium phosphate nanocluster of claim 45 wherein a phosphate centre of the phosphopeptide or phosphoprotein comprises an amino acid sequence of at least one of: ELEELNVPGADDSSSSDDDDDDDRINKK (SEQ ID NO 2), ELEELNVPGADDSSSDDDSDDDDRINKK (SEQ ID NO 3), or MRELEELNVPGADDSSSDDDSDDDDRINKKIEDPNSSSVDKLAAA LEHHHHHH (SEQ ID NO 4).

48. A thermodynamically stable calcium phosphate nanocluster as claimed in claim 45 having a core radius greater than or equal to 3 nm and/or wherein the core surface area per phosphate centre in the nanocluster is less than 0.6 nm².

49. A method of providing a thermodynamically stable calcium phosphate nanocluster, comprising the step of:
preparing a nanocluster forming solution, wherein the nanocluster forming solution is prepared by mixing of calcium ions, phosphate ions and phosphopeptide or phosphoprotein, and
wherein the phosphopeptide or phosphoprotein comprises at least one of a) a recombinantly expressed phosphopeptide wherein said recombinantly expressed phosphopeptide; i) includes a phosphate centre modified such that at least one of a phosphorylated residue and an acidic residue or combinations of these residues is increased within the phosphate centre such that the phosphate centre has increased calcium phosphate sequestering power, or ii) is modified such that the modified recombinantly expressed phosphopeptide includes an increased number of discrete phosphate centres in comparison to a non-modified version of the recombinantly expressed phosphopeptide, or iii) is modified such that the modified recombinant phosphopeptide has increased calcium phosphate sequestering power over a non-modified version of the recombinantly expressed phosphopeptide, preferably by alteration of the number and/or type and/or phosphorylation of the amino acid residues or spacing of particular amino acid residues within a phosphate centre, or removing amino acid sequences which promote the conversion of amorphous calcium phosphate into a more crystalline phase such as apatite, b) a calcium binding phosphoprotein/phosphopeptide, or a variant or a fragment thereof wherein the phosphopeptide or phosphoprotein does not include an individual casein or mixture of caseins or enzymatic digests of an individual casein or a mixture of caseins, or c) a combination of a) and b).

50. The method of claim 49 wherein the phosphopeptide or phosphoprotein comprises an amino acid sequence of at least one member selected from the group consisting of fetuin A (SEQ ID NO 10), proline-rich basic phosphoprotein 4 (SEQ ID NO 11), matrix Gla protein (SEQ ID NO 12), secreted phosphoprotein 24 (SEQ ID NO 13), osteopontin (SEQ ID NO 15), integrin binding sialophosphoprotein (SEQ ID NO 16), matrix extracellular bone phosphoglycoprotein (SEQ ID NO 17), dentin matrix acidic phosphoprotein 1 (SEQ ID NO 18) or variants or fragments thereof.

51. The method of claim 49 wherein a phosphate centre of the phosphopeptide or phosphoprotein comprises at least one of:
(SEQ ID NO 2)
ELEELNVPGADDSSSSDDDDDDDRINKK,
(SEQ ID NO 3)
ELEELNVPGADDSSSDDDSDDDDRINKK,
or
(SEQ ID NO 4)
MRELEELNVPGADDSSSDDDSDDDDRINKKIEDPNSSSVDKLAAA
LEHHHHHH.

52. The method of claim 49 wherein, in the step of preparing a nanocluster forming solution, the pH of the nanocluster forming solution is raised to form the calcium phosphate nanoclusters by generating ammonia homogeneously in the solution to gently raise the pH by the catalytic hydrolysis of urea in the solution by urease.

53. A thermodynamically stable calcium phosphate nanocluster obtainable using the method as claimed by claim 49.

54. A thermodynamically stable calcium phosphate nanocluster obtainable using the method of claim 49 wherein the nanocluster has a core radius greater than or equal to 3 nm and/or wherein the core surface area per phosphate centre in the nanocluster is less than 0.6 nm².

55. A phosphopeptide or phosphoprotein for use in the method of claim 49 wherein
(a) the phosphopeptide comprises a secretory calcium binding phosphopeptide or phosphoprotein, or a variant or a fragment thereof, which does not include residues which promote the conversion of amorphous calcium phosphate (ACP) into a more crystalline phase such as apatite thereof excluding any individual casein or mixture of caseins or enzymatic digests of any individual casein or mixture of caseins;
(b) the phosphopeptide comprises a recombinant phosphopeptide or phosphoprotein or combination thereof wherein said recombinant phosphopeptide or phosphoprotein i) includes a phosphate centre modified to provide an increased number of phosphorylated residues within the phosphate centre such that the phosphate centre has increased calcium phosphate sequestering power, or ii) is modified to include an increased number of discrete phosphate centres over a non-modified recombinantly expressed phosphopeptide, or iii) includes a modification to allow for increased calcium phosphate sequestration.

56. A phosphophopeptide as claimed in claim 55 wherein the phosphopeptide comprises an amino acids sequence of at least one of:
(SEQ ID NO 2)
ELEELNVPGADDSSSSDDDDDDDRINKK,
(SEQ ID NO 3)
ELEELNVPGADDSSSDDDSDDDDRINKK,
and
(SEQ ID NO 4)
MRELEELNVPGADDSSSDDDSDDDDRINKKIEDPNSSSVDKLAAA
LEHHHHHH.

57. A formulation, a pharmaceutical composition, adjuvant, artificial biofluid for use as at least one of a blood, blood plasma, extracellular and lymphatic fluids, synovial fluid, cerebrospinal fluid, urine and saliva substitute, comprising a thermodynamically stable calcium phosphate nanocluster of claim 45.

58. Use of a thermodynamically stable calcium phosphate nanocluster of claim 45 in medicine, for the inhibition or prevention of mineralised tissue demineralisation, for the treatment or prevention of pathological calcification, in a food or beverage, in a natural or synthetic fluid to maintain the stability and degree of supersatuation of the fluid, in an artificial biofluid.
